SEQUENCE LISTING (71 sequences)

SEQ ID NO: 1 - protein, 31 aa, Artificial Sequence
  partial protein sequence of Clitopilus passeckerianus
GNFMATPSTTAAYLMKATKWDDRAEDYLRHV

SEQ ID NO: 2 - protein, 29 aa, Artificial Sequence
  partial protein sequence of Clitopilus passeckerianus
FEAPTYFRCYSFERNASVTVNSNCLMSLL

SEQ ID NO: 3 - protein, 26 aa, Artificial Sequence
  partial protein sequence of Clitopilus passeckerianus
RLANDLHSISRDFNEVNLNSIMFSEF

SEQ ID NO: 4 - protein, 30 aa, Artificial Sequence
  partial protein sequence of Clitopilus passeckerianus
DYINIIRVTYLHTALYDDLGRLTRADISNA

SEQ ID NO: 5 - protein, 31 aa, Artificial Sequence
  partial protein sequence of Clitopilus passeckerianus
YSLLNHPRAQLASDNDKGLLRSEIEHYFLAG

SEQ ID NO: 6 - protein, 28 aa, Artificial Sequence
  partial protein sequence of Clitopilus passeckerianus
SHYRWTHVVGADNVAGTIALVFALCLLG

SEQ ID NO: 7 - protein, 35 aa, Artificial Sequence
  partial protein sequence of Clitopilus passeckerianus
PSSTFAKVEKGAAGKWFEFLPYMTIAPSSLEGTPI

SEQ ID NO: 8 - DNA, 2880 bp, Clitopilus passeckerianus
  CDS (1)..(2880)
atgggtctatccgaagatcttcatgcacgcgcccgaaccctcatgcagactctcgagtctgcgctcaatacgccaggttctaggggtattggcaccgcgaatccgactatctacgacactgcttgggtagccatggtctcccgtgagatcgacggcaaacaagtcttcgtcttcccagagaccttcacctacatctacgagcaccaggaggctgacggcagttggtcaggggatggatccctcattgactccatcgtcaatactctggcctgccttgtcgctctcaagatgcacgagagcaacgcctcaaaacccgacatacctgcccgtgccagagccgctcaaaattatctcgacgatgccctaaagcgctgggacatcatggagactgagcgtgtcgcgtacgagatgatcgtaccctgcctcctcaaacaactcgacgcctttggcgtatccttcaccttcccccatcatgacctcctgtacaacatgtacgccggaaagctggcgaagcttaactgggaggctatctacgccaagaacagctctttgcttcactgcatggaggcattcgtcggtgtctgcgacttcgatcgcatgcctcatctcctacgtgatggtaacttcatggctacgccatccaccaccgctgcgtacctcatgaaggccactaagtgggatgaccgagcggaagattaccttcgccacgttatcgaggtctacgcaccccatggccgagatgttgttcctaatctctggccgatgaccttcttcgagatcgtatggtccctcagctccctttatgacaacaacctcgaatttgcacaaatggatccggaatgcttggatcgcattgccctcaaattacgtgaattccttgtggcaggaaaaggtgtcttaggctttgttcccggaaccactcacgacgctgacatgagctcgaaaaccctgatgctcttacaagttctcaaccacccatactcccatgacgaattcgttacagagtttgaggcacctacctacttccgttgctactccttcgaaaggaacgcaagcgtgaccgtcaactccaactgccttatgtcgctcctccacgcccctgatgtcaacaagtacgaatcccaaatcgtcaagatcgccacttatgtcgccgatgtctggtggacatcagcaggtgtcgtcaaagacaaatggaatgtatcagaatggtactcctccatgctgtcctcacaggcgcttgtccgtctccttttcgagcacggaaagggcaaccttaaatccatatccgagaagctcctgtctagggtgtccatcgcctgcttcacgatgatcagtcgtattctccagagccagaagcccgatggctcttggggatgcgctgaagaaacctcatacgctctcattacactcgccaacgtcgcttctcttcccacttgcgacctcatccgcgaccacctgtacaaagtcattgaatccgcgaaggcatacctcacccccatcttctacgcccgccctgctgccaaaccggaggaccgtgtctggattgacaaggttacatacagcgtcgagtcattccgcgatgcctaccttgtttctgctctcaacgtacccatcccccgcttcgatccatcttccatcagcactcttcctgctatctcgcaaaccttgccaaaggaactctctaagttcttcgggcgtcttgacatgttcaagcctgctcctgaatggcgcaagcttacgtggggcattgaggccactctcatgggccccgagcttaaccgtgttccatcgtccacgttcgccaaggtagagaagggagcggcgggcaaatggttcgagttcttgccatacatgaccatcgctccaagtagcttggaaggcactcctatcagttcacaagggatgctggacgtgctcgttctcatccgcggtctttacaacaccgacgactacctcgatatgaccctcatcaaggccaccaatgaggacttagacgatctcaagaagaagatccgcgacctattcgcggatccgaagtcgttctcgaccctcagcgaggtcccggatgaccggatgcctacgcacatcgaggtcattgagcgctttgcctattccctgttgaaccatcctcgtgcgcagctcgccagcgataacgataagggtctcctccgctccgaaattgagcactatttcctggcaggtattgctcagtgcgaagaaaacattctccttcgtgaacgtggactcgacaaggagcgcatcggaacctctcactatcgctggacacatgtcgttggcgctgataacgtcgctgggaccatcgccctcgtcttcgccctttgtcttcttggtcatcagatcaatgaagaacgaggctctcgcgatttggtggacgttttcccctccccagtcctgaagtacttgttcaacgactgtgtcatgcactttggtacattctcaaggctcgccaacgaccttcacagtatctcccgcgacttcaacgaagtcaatctcaactccatcatgttctccgaattcaccggaccaaagtctggtaccgatacagagaaggctcgtgaagctgctctgcttgaattgaccaaattcgaacgcaaggctaccgacgatggtttcgagtacttggtccagcaactcactccacatgtcggggccaaacgcgcacgggattatatcaatataatccgcgtcacctacctgcacacggccctctacgatgacctcggtcgtctcactcgtgcagatatcagcaacgccaaccaggaggtgtccaaaggtaccaatggggtcaagaaaatcaatgggtcatcgacaaatgggaccaaggtcacagcaaatgggagcaatggaatccaccattga

SEQ ID NO: 9 - protein, 959 aa, Clitopilus passeckerianus
MGLSEDLHARARTLMQTLESALNTPGSRGIGTANPTIYDTAWVAMVSREIDGKQVFVFPETFTYIYEHQEADGSWSGDGSLIDSIVNTLACLVALKMHESNASKPDIPARARAAQNYLDDALKRWDIMETERVAYEMIVPCLLKQLDAFGVSFTFPHHDLLYNMYAGKLAKLNWEAIYAKNSSLLHCMEAFVGVCDFDRMPHLLRDGNFMATPSTTAAYLMKATKWDDRAEDYLRHVIEVYAPHGRDVVPNLWPMTFFEIVWSLSSLYDNNLEFAQMDPECLDRIALKLREFLVAGKGVLGFVPGTTHDADMSSKTLMLLQVLNHPYSHDEFVTEFEAPTYFRCYSFERNASVTVNSNCLMSLLHAPDVNKYESQIVKIATYVADVWWTSAGVVKDKWNVSEWYSSMLSSQALVRLLFEHGKGNLKSISEKLLSRVSIACFTMISRILQSQKPDGSWGCAEETSYALITLANVASLPTCDLIRDHLYKVIESAKAYLTPIFYARPAAKPEDRVWIDKVTYSVESFRDAYLVSALNVPIPRFDPSSISTLPAISQTLPKELSKFFGRLDMFKPAPEWRKLTWGIEATLMGPELNRVPSSTFAKVEKGAAGKWFEFLPYMTIAPSSLEGTPISSQGMLDVLVLIRGLYNTDDYLDMTLIKATNEDLDDLKKKIRDLFADPKSFSTLSEVPDDRMPTHIEVIERFAYSLLNHPRAQLASDNDKGLLRSEIEHYFLAGIAQCEENILLRERGLDKERIGTSHYRWTHVVGADNVAGTIALVFALCLLGHQINEERGSRDLVDVFPSPVLKYLFNDCVMHFGTFSRLANDLHSISRDFNEVNLNSIMFSEFTGPKSGTDTEKAREAALLELTKFERKATDDGFEYLVQQLTPHVGAKRARDYINIIRVTYLHTALYDDLGRLTRADISNANQEVSKGTNGVKKINGSSTNGTKVTANGSNGIHH

SEQ ID NO: 10 - DNA, 93 bp, Artificial Sequence
  partial nucleic acid sequence of Clitopilus passeckerianus
ggtaacttcatggctacgccatccaccaccgctgcgtacctcatgaaggccactaagtgggatgaccgagcggaagattaccttcgccacgtt

SEQ ID NO: 11 - DNA, 87 bp, Artificial Sequence
  partial nucleic acid sequence of Clitopilus passeckerianus
tttgaggcacctacctacttccgttgctactccttcgaaaggaacgcaagcgtgaccgtcaactccaactgccttatgtcgctcctc

SEQ ID NO: 12 - DNA, 78 bp, Artificial Sequence
  partial nucleic acid sequence of Clitopilus passeckerianus
aggctcgccaacgaccttcacagtatctcccgcgacttcaacgaagtcaatctcaactccatcatgttctccgaattc

SEQ ID NO: 13 - DNA, 3041 bp, Clitopilus passeckerianus
  CDS (1)..(786)
  CDS (841)..(961)
  CDS (1017)..(1303)
  CDS (1356)..(3038)
atgggtctatccgaagatcttcatgcacgcgcccgaaccctcatgcagactctcgagtctgcgctcaatacgccaggttctaggggtattggcaccgcgaatccgactatctacgacactgcttgggtagccatggtctcccgtgagatcgacggcaaacaagtcttcgtcttcccagagaccttcacctacatctacgagcaccaggaggctgacggcagttggtcaggggatggatccctcattgactccatcgtcaatactctggcctgccttgtcgctctcaagatgcacgagagcaacgcctcaaaacccgacatacctgcccgtgccagagccgctcaaaattatctcgacgatgccctaaagcgctgggacatcatggagactgagcgtgtcgcgtacgagatgatcgtaccctgcctcctcaaacaactcgacgcctttggcgtatccttcaccttcccccatcatgacctcctgtacaacatgtacgccggaaagctggcgaagcttaactgggaggctatctacgccaagaacagctctttgcttcactgcatggaggcattcgtcggtgtctgcgacttcgatcgcatgcctcatctcctacgtgatggtaacttcatggctacgccatccaccaccgctgcgtacctcatgaaggccactaagtgggatgaccgagcggaagattaccttcgccacgttatcgaggtctacgcaccccatggccgagatgttgttcctaatctctggccgatgaccttcttcgagatcgtatgggtatgttctctcattatttatttactaactcagtactaactacctggtttccagtccctcagctccctttatgacaacaacctcgaatttgcacaaatggatccggaatgcttggatcgcattgccctcaaattacgtgaattccttgtggcaggaaaaggtgtcttaggctttggtcagtccttcttcgagcattttgacgtatcatggctgatgaacgacctggatagttcccggaaccactcacgacgctgacatgagctcgaaaaccctgatgctcttacaagttctcaaccacccatactcccatgacgaattcgttacagagtttgaggcacctacctacttccgttgctactccttcgaaaggaacgcaagcgtgaccgtcaactccaactgccttatgtcgctcctccacgcccctgatgtcaacaagtacgaatcccaaatcgtcaagatcgccacttatgtcgccgatgtctggtggacatcagcaggtgtcgtcaaagacaaatgggtaagccataccttatcaattgatctgactgtcaactaaactatcctttcagaatgtatcagaatggtactcctccatgctgtcctcacaggcgcttgtccgtctccttttcgagcacggaaagggcaaccttaaatccatatccgagaagctcctgtctagggtgtccatcgcctgcttcacgatgatcagtcgtattctccagagccagaagcccgatggctcttggggatgcgctgaagaaacctcatacgctctcattacactcgccaacgtcgcttctcttcccacttgcgacctcatccgcgaccacctgtacaaagtcattgaatccgcgaaggcatacctcacccccatcttctacgcccgccctgctgccaaaccggaggaccgtgtctggattgacaaggttacatacagcgtcgagtcattccgcgatgcctaccttgtttctgctctcaacgtacccatcccccgcttcgatccatcttccatcagcactcttcctgctatctcgcaaaccttgccaaaggaactctctaagttcttcgggcgtcttgacatgttcaagcctgctcctgaatggcgcaagcttacgtggggcattgaggccactctcatgggccccgagcttaaccgtgttccatcgtccacgttcgccaaggtagagaagggagcggcgggcaaatggttcgagttcttgccatacatgaccatcgctccaagtagcttggaaggcactcctatcagttcacaagggatgctggacgtgctcgttctcatccgcggtctttacaacaccgacgactacctcgatatgaccctcatcaaggccaccaatgaggacttagacgatctcaagaagaagatccgcgacctattcgcggatccgaagtcgttctcgaccctcagcgaggtcccggatgaccggatgcctacgcacatcgaggtcattgagcgctttgcctattccctgttgaaccatcctcgtgcgcagctcgccagcgataacgataagggtctcctccgctccgaaattgagcactatttcctggcaggtattgctcagtgcgaagaaaacattctccttcgtgaacgtggactcgacaaggagcgcatcggaacctctcactatcgctggacacatgtcgttggcgctgataacgtcgctgggaccatcgccctcgtcttcgccctttgtcttcttggtcatcagatcaatgaagaacgaggctctcgcgatttggtggacgttttcccctccccagtcctgaagtacttgttcaacgactgtgtcatgcactttggtacattctcaaggctcgccaacgaccttcacagtatctcccgcgacttcaacgaagtcaatctcaactccatcatgttctccgaattcaccggaccaaagtctggtaccgatacagagaaggctcgtgaagctgctctgcttgaattgaccaaattcgaacgcaaggctaccgacgatggtttcgagtacttggtccagcaactcactccacatgtcggggccaaacgcgcacgggattatatcaatataatccgcgtcacctacctgcacacggccctctacgatgacctcggtcgtctcactcgtgcagatatcagcaacgccaaccaggaggtgtccaaaggtaccaatggggtcaagaaaatcaatgggtcatcgacaaatgggaccaaggtcacagcaaatgggagcaatggaatccaccattga

SEQ ID NO: 14 - protein, 959 aa, Clitopilus passeckerianus; identical to SEQ ID NO: 9

SEQ ID NO: 15 - DNA, 27050 bp, Clitopilus passeckerianus
cgacatgccaaggtatattggcgtcagggcagaatagatagaagtttaagctctaaacgagattgacaccatgcggcccaccctccacttcaggccggttcatgtaatggcaacaccgagttgacctccgagcagttcgtgggctcaatccacttgagctagttcgtcgatcgactgagccaggacgacatacattctgtgattgcatccgagtcacaagcgctgccagcgtctcctacgtggaaaaactgcggatggcgcatgacagccactgcctcctctggtcgtatcacgctagcctgcgtgacgtgcattgttatcgggtggcaataagtctgataacgatcccgaatgggatcttggctaggtggactgtcatgcctcatatgcatgccaactttacccaattggtgcttacagatatatctgtgttcgcacactccacctcgcattgtccacgacctccaccttgacattcttcgagactcttcccaacatctatggctccgtcaacggaacgtgctctaccagtccttgtaatatggactgctataggcttggcctactggatagactctcagaagaagaaaaagcagcacctgccgcctgggccaaagaaacttccaattatcggcaacgtgatggacctaccggcgaaggtcgaatgggaaacctatgctcgctggggtaaagagtacagtacgtcgactctatgttagaatgacgcccgtagactcattgaagccttctgaaaatagactctgatatcatacatgttagcgccatgggaacctcgatcgtaatactgaattctgccaacgccgccaatgacttgttgctgaagaggtcggcgatctactcgagcaggtatggtttcagcacggtattgccaatgtctacctgacacgctctatagaccacacagcacgatgcaccacgaactgtaagtatgctgttcgctacaaattagcactgaagattcacatcacgttaccaggtcaggctggggctttacgtgggccttaatgccttatggcgagtcatggcgggctggtcgtagaagcttcaccaagcacttcaactcttcaaaccccggtataaaccaacctcgtgagttgcgatatgtgaaacggttcctcaagcagctttacgagaagcccgacgacgttctcgatcatgtacggaagtatgtttttcgacaggtctttcgatgagccataaacctcatttctttgacagcttggtcggctctacaacgctttcaatgacctatgggctcgagactgaaccttacaacgatccctatgttgacctggtagagaaagctgtccttgcagcgtctgagattatgacatctggcgcctttcttgttgacatcatccctgccatgaaacacattcctccatgggtcccagggactatcttccatcaaaaggccgccttaatgcgaggtcatgcgtactatgttcgtgaacagccattcaaagttgcccaggatatgattgtaagcaaccttgcccaactttgtccattcccttgcctaattcattcgtacttagaaaactggtgactatgagccctcctttgtatccgacgctcttcgagatcttgagaactcggaaaaccaggatgaagatttagagcacctcaaggatgttgctggtcaagtctacattggtatatcatgcctttctcttcggtcgtggatgcctctaattgttgactgtttagctggtgctgatacgactgcatccgccttgggcaccttcttcctcgccatggtctgtttccccgaagtacagaaaaaagcacaacgagaattggatagtgttctcaatggaaggatgcccgagcacgtcgacttcccatctttcccatacctcaacgctgtgatcaaagaagtctaccggtgtgttatttatgcgtcgagcgcggggtttaggtcggctgacgttcgtgatgcagctggagacctgtgactcctatgggcgtacctcatcaaaccatctcagatgacgtttacagggactaccacatccctaagggatccatcgtgttcgccaaccaatggtatgtttgcgttcttgacttccgtattccaatcttgacttgtcttaagggcaatgtccaacgacgagaccgattacccccagccggacgaattccagcctgagcgatacttgaccgaagatggcaagcctaataaggcggttagagacccctttgatattgcgttcggcttcggtagaaggtcagcaactattcattgagctgcgccgaggatactgacctcgccttttagaatttgcgctggtcgttaccttgctcattccaccatcaccttggctgcagcctctgttctgtcgctatttgatctcttaaaagcagttgacgaaaatggcaaggaaatcgagcctactagagagtatcatcaggctatgatttcgtgagtgatttaattcgctgctgaacacccggccctggctaaacgccgtctgcagacgtccacttgatttcccgtgtcgtatcaagccaaggagcaaggaagccgaggaggttatccgtgcttgcccgttaacgttcaccaagccaactcttggtgtctagacacatgtttacatcttcgaacgtgtatatcagaatggaatctcttgtattcgttgaacacgcgtttgatcagaaaatctgagataacgacttcaaggttcaatctccaacctccgcatgtagtgaggataataccttcagcgaagatggtcttgtctcgccatggccttattcagtctcgggaacctatgcgttcaagttaagagtttatttacaaacatcgaatgaaagacagctaatgggcgtgcctactgtactacacgaggaggattccattctccccggtacagaccaagaatgacactactgtccaacgacatcgaagccttgccccttcctgagacagtcgcccccagccaattcggacagctatagacgaaccatgcgacagtccaaagatgtccaagggccttccagtaccaggccggtttaataccgagcttccttccgacccagagaaccccatcctccatctgtatgatcgccgcttggacgcagaagaactccatagtcccggccttgtgccaatctgcgtgaaccttgaaatccccaatcgcatgcaaaactcccgagaggaagaatgcggtgtacagttgtacgtaaagcgccgggttagagccagacttcaagcccagcatcttcgtcgaaagatgttttcctggggctgataggcacttattaggtttaggtcagagagggtgatgatgattgaacagaagaagcttactctgcgaactaattgatgccaggaccgacttttcattgatatgagataaggtttacccaggaatagattagatggacttacccccagaaccgcctaacggtagaagcatctctccagcggccaaacacggggacccagtcctatcagtagctctaatcagaccctagacctttcagtctggcgcagttgacaattacacacctgtggttctgagtagcccgagagaacttgcatgacactcaatgccgtaagaagaattgatacttggtttgtcgtgaacaggagccatgcggcaatatctgcacaccgccagatcaaagggcgagaagcaagaggttcgatgaatgcgactgggcttcggctatacagcgagaccagatcacaaataaggtaatgccgaaaacctgtgacgagttgctggaggagaaatgaaaggcgtgaagtctttggggtgaacctgtggacgaaaatcttcggctcccaattccagccaacgccacgccgactttgcaccagccatgtcgcccacttgatgcgttcaagcaacgaggcttgctcgacacctttctggttgcggaattgcaattctctctggacgtccgtcaagagaatataatccgtggccgtgagggtaataacgaagaggttgttggcaatgtcataatccaaggtcgaatcgccggtcgtggtcatggtgaggtaggtgttaagcccaacaataataacccagagaacccatcgtcctctagcaggccgaatggcacaagagagtaccaatagaatgaaagatagaaccagaagttccggcgagaagggcttcatgctggtatcagtccaaggaagacggcctcgctggggaggatccttgtcggtgtcgcagcattcttcaagcagatcctcacttttgagaccgctccgagcggaaaacacgagtcaaggccaaagatgataagatattacaatgcaacactcttggtacagttctgtcaccgagcatgtggtgcaccaggttgatacggtggccacggtaaaacaatgggaaaaccggcgaccgataccaccaggggtttccattcggcgtggacaagagattgtattacgacccgaagtcgccacctttcgtcttagctgtcagtttctgtccagtttgctcctcatggcaaatcgctagtcccagcctccttgaatccacaccgccggcccggagctatgtattaacctggttcaagtctgtttcgaaccagcaatggagcaagtccagcttctgtgtaaggcactgatgaatgtcaatctccaacctgtcgatcttcgaagctcgatcaggattacccccaatcagtattgttacaaagatagcacacgtccaggtccatcgcgggtaaattttaatttcccctcggccgtgggtctttgcgtaagccattctgcgggtacggtacccacctaagtccaaagaaaacctcataccgagactgatagagatccaacgaacggagatactgttcttgggcctgcgtcatacatggaagacaggagtgtttccttttcattaaatgtcaaaccaatagctgtgttgaaccaagtcgatttcttttccgagcttgagaacttgaagtatgaaataaaaatacatttgcatctatcagaaaggcgatatcttagagctacgtgtatagtctaccgaactagcgatatcaatggtggattccattgctcccatttgctgtgaccttggtcccatttgtcgatgacccattgattttcttgaccccattggtacctttggacacctcctggttggcgttgctgatatctgcacgagtgagacgaccgaggtcatcgtagagggccgtgtgcaggtaggtgacgcggattatattgatataatcccgtgcgcgtttggccccgacatgtggagtgagttgctggaccaagtactcgaaaccatcgtcggtagccttgcgttcgaatttggtcaattcaagcagagcagcttcacgagccttctctgtatcggtaccagactttggtccggtgaattcggagaacatgatggagttgagattgacttcgttgaagtcgcgggagatactgtgaaggtcgttggcgagccttgagaatgtaccaaagtgcatgacacagtcgttgaacaagtacttcaggactggggaggggaaaacgtccaccaaatcgcgagagcctcgttcttcattgatctgatgaccaagaagacaaagggcgaagacgagggcgatggtcccagcgacgttatcagcgccaacgacatgtgtccagcgatagtgagaggttccgatgcgctccttgtcgagtccacgttcacgaaggagaatgttttcttcgcactgagcaatacctgccaggaaatagtgctcaatttcggagcggaggagacccttatcgttatcgctggcgagctgcgcacgaggatggttcaacagggaataggcaaagcgctcaatgacctcgatgtgcgtaggcatccggtcatccgggacctcgctgagggtcgagaacgacttcggatccgcgaataggtcgcggatcttcttcttgagatcgtctaagtcctcattggtggccttgatgagggtcatatcgaggtagtcgtcggtgttgtaaagaccgcggatgagaacgagcacgtccagcatcccttgtgaactgataggagtgccttccaagctacttggagcgatggtcatgtatggcaagaactcgaaccatttgcccgccgctcccttctctaccttggcgaacgtggacgatggaacacggttaagctcggggcccatgagagtggcctcaatgccccacgtaagcttgcgccattcaggagcaggcttgaacatgtcaagacgcccgaagaacttagagagttcctttggcaaggtttgcgagatagcaggaagagtgctgatggaagatggatcgaagcgggggatgggtacgttgagagcagaaacaaggtaggcatcgcggaatgactcgacgctgtatgtaaccttgtcaatccagacacggtcctccggtttggcagcagggcgggcgtagaagatgggggtgaggtatgccttcgcggattcaatgactttgtacaggtggtcgcggatgaggtcgcaagtgggaagagaagcgacgttggcgagtgtaatgagagcgtatgaggtttcttcagcgcatccccaagagccatcgggcttctggctctggagaatacgactgatcatcgtgaagcaggcgatggacaccctagacaggagcttctcggatatggatttaaggttgccctttccgtgctcgaaaaggagacggacaagcgcctgtgaggacagcatggaggagtaccattctgatacattctgaaaggatagtttagttgacagtcagatcaattgataaggtatggcttacccatttgtctttgacgacacctgctgatgtccaccagacatcggcgacataagtggcgatcttgacgatttgggattcgtacttgttgacatcaggggcgtggaggagcgacataaggcagttggagttgacggtcacgcttgcgttcctttcgaaggagtagcaacggaagtaggtaggtgcctcaaactctgtaacgaattcgtcatgggagtatgggtggttgagaacttgtaagagcatcagggttttcgagctcatgtcagcgtcgtgagtggttccgggaactatccaggtcgttcatcagccatgatacgtcaaaatgctcgaagaaggactgaccaaagcctaagacaccttttcctgccacaaggaattcacgtaatttgagggcaatgcgatccaagcattccggatccatttgtgcaaattcgaggttgttgtcataaagggagctgagggactggaaaccaggtagttagtactgagttagtaaataaataatgagagaacatacccatacgatctcgaagaaggtcatcggccagagattaggaacaacatctcggccatggggtgcgtagacctcgataacgtggcgaaggtaatcttccgctcggtcatcccacttagtggccttcatgaggtacgcagcggtggtggatggcgtagccatgaagttaccatcacgtaggagatgaggcatgcgatcgaagtcgcagacaccgacgaatgcctccatgcagtgaagcaaagagctgttcttggcgtagatagcctcccagttaagcttcgccagctttccggcgtacatgttgtacaggaggtcatgatgggggaaggtgaaggatacgccaaaggcgtcgagttgtttgaggaggcagggtacgatcatctcgtacgcgacacgctcagtctccatgatgtcccagcgctttagggcatcgtcgagataattttgagcggctctggcacgggcaggtatgtcgggttttgaggcgttgctctcgtgcatcttgagagcgacaaggcaggccagagtattgacgatggagtcaatgagggatccatcccctgaccaactgccgtcagcctcctggtgctcgtagatgtaggtgaaggtctctgggaagacgaagacttgtttgccgtcgatctcacgggagaccatggctacccaagcagtgtcgtagatagtcggattcgcggtgccaatacccctagaacctggcgtattgagcgcagactcgagagtctgcatgagggttcgggcgcgtgcatgaagatcttcggatagacccatagtgaaggtgagagcgcagcgaagtaaagggagtctgggttggaaaggtggtgagtctgggttcaagtactggaacgaagacgggatcttacttttaagcagtctctggactttgcatattgggcaaaagctccagtcaaccaaaccacaagttgtttggttgaagctgacgtcttggggtcacatcacccgactggtttggctcagagttcaagctcgttatccgtgaaacaagtgtccactcgaaggagttggacaggttctagatgcgtaccaacatggttgcaccaataatggttcagccgatatcagaaccgacagaaaatcccgcaggcatgtcaagattccgatgttccacaattatcagaacccggctatgtcaattgtgcgacgcgttatggccaagattgggccaagagctaaggttccaacgagaaggtgtcactggaagtccaggaaaggcaaacgaacccgttcgagaagatttaggcttgtagaagaaccagctgactaagcccatcactcttcattttggcgtactgtgtactcggggcaaccactttcatagtgccaaccatctgccgtcttcaccggcggcctatctcttaagaaatctaagatttggagataataagtacaagattcgtgctaagcggtttctgacgacctggcttagctccgtggttcctggatcttctttgcctattccattttgcatccggacattcgccatatcctcccgattctgtcagagcttggccccgctcatgacccctcaaacacaagggtcttgcgaattggggcactctgactacatctgtcaactcgatacctttctaggctatacgctgttagaactcagaggatatttcttatttatctattcactgagccgaagtgtaccaattagcattgcccactccttcagatttacctccctaaattcatacgatgagaatacctaacgtctttctctcttacctgcgacaagtcgccgtcgacggcactctgtcatcttgttctggagtgaaatcacgaaagccggtcattgcctatggctttgacgactcacaagactctctcgtcgatgtaagcaccttcttctttatcatttcaactctggctcaccggcttggtaaaaacctaggagaatgacgaaaaaatattggagccctttggctactatcgtcatcttttgaaaggcaagagcgccaggacagtgttgatgcactgcttcaacgcgttccttggactgcccgaagattgggtcattggcgtaacaaaggccattgaagaccttcataatgcatccctactgtgagcataatgtccacactatttttttttcgttcgatctctgacatcgcacctggcagaattgatgacatcgaagacgagtccgctctccgtcgtggttcaccagccgcccacatgaagtacgggattgccctgaccatgaacgcggggaatcttgtctacttcacggtccttcaagacgtctatgacctcggaatgaagacaggcggcactcaggtcgccaacgcaatggctcgcatctacactgaagagatgattgagctccaccgtggtcaaggcattgaaatctggtggcgtgaccagcggtcccctccttccgtcgatcaatacattcacatgctcgagcagagtgagtttttccaccgactgctgtcatccacggacatatcctgactattccctcaccagaaaccggcggcctgctcaggcttggcgtacggctcttgcaatgccatcccggtgtcaataacagggccgacctctccgacattgcgctccgtattggtgtctactaccaacttcgcgacgactacatcaacctcatgtccacaagctaccatgacgagcgtggattcgctgaggacataaccgaaggaaagtacactttcccgatgttacactcactcaagaggtcacctgattctggactgcgtggtatgtgttcagcagtcgcttgctttcaatgatttactgacagcccgggatttcatttagaaatcttggaccttaaaccggcagacattgccctgaagaagaaagctatcgctatcatgcaagatactggatcgcttgttgcaacccggaaccttctcggtgcagttaagaatgatctcagtggattggttgctgaacagcgtggagacgactacgctatgagcgcgggtcttgaacgattcttggaaaagttgtacatcgcagagtagataagaatctcaatagaattcgtccatgaatggaacaatatttaaatccaattcatctcaaggctcctcgcaattgaatccaaatttaaaattaaatactcgatttttccagatgttattgcaaccaaaacatctcgacgtcacaggttcgccacagcctcggtaaagggaatttgagggtatgatgcatatgttgatccacctttgagtggtgcccctcgacagacaatcatccttcctttatctcctttcagcagacccagcctaaaccggggtagctattgacgactgcaagttcagcgtggaagttcttgacgatggcattatctcacatcacatgggaaacaagacattgagaactgaacattcgtacattacgaaactctctatcgccgaatactatggtgttaggaggctacaacgcagcgaacgcttccttaatcaagtcttccttcatcttatctcgaggttcaattttgcatgcgaacggaagtggaagagtctcaagagaaacctgtcagaataaaagtgatcgttaacctcggcagccgtatcttgagaaaatgataagcgactatacgcaccgacttgtcgtaacagtcctccatgttcatattcttcacagtgtccttgtttgaagaatctgggtaaaaattgaatgcccaacagagcctcatgatgaagagaccctgtgccagttcatatcagcaaatttcttcgacccggaagaacacctaacgtacagttgatcgttttgccagcttatcgcctgggcagactctctaaagacagtaaatgatgaggtaccgagtgaaggataggatgagactcacacgtccagcaccgaacaggaaatcgggattgacgtcttcagataagcctggcttcgtgccgtttggcgacaagaaatagcgttcaggcttgaaggcctcaggttcgtcgaagagctctggaggttagtgtcgtcggaaacacgtagttaatcgagttgggatcagggacttaccggggtcatggcccattccccctagagactggtgcgttcaatgctcagaaatgtgagagataatcaactcacagatgttcatgaagatcatacttccctctggcagtacgtaaccgccatactaggaagcccgtcagtgtccgaaaatcacgataagataccacgttcgtgaactcacagacaagctctcccgcgagacgtggggaagggctacagggccgactggccgaagccgaaggacctaagccacgcgattgagaacaatgaaactgacagatgtcttcccatgggactcacctcctgtaggaacgccttgagataaggcaaccgttccaaatcattgaagcatggcatggtttcggtccccaaaacattgtccagctcgtcctgtatcttgcgctggcagtccgggtgggcgataagagcaagaatacacgattcgatgtacgatatcgtggtcttcgcgccggcgtccaagaaaccaccgctaaggtttctagaaaatattatcagatactcgctcaacgtatcagcagaggatttacgtacgataactcaagccagctacgaccatccggatggtcaatcacggactctgcaaaagatccggtcctgaccccggaatccatcgccttcttggcaccttccaagagagaattgtagacaccattacggaaatccttgaattcatccacaatggtcttccagccggccccggggaaaccgcgagggatgtagtctaagaaggggaaagcgtcgaccgctgcaccgttgtgagcgatttgaccaattctggtggcagcttcgtatgcattctcgataatcgtgccatagtaactttcgcaacgtggctggccatacacaatgtgcaggagtagcgacatcatagcacgcctaatatggatcggccgattctatcgttagttcgtcagttaaggacatcactaaccagaaagattcgtcgaactgacaggagcgtccatcaatagatcgtgcatgaggttcacagattcctcttcttgtcgcggtatgtagccactcaaggcacttggcgttaggtaattgtggatacctttgcgaccagtcttccatacggaagtgtctgaagaaaatgcgttatcaggaagtgtttaaacggtgtagagaaatgacatacccatgctttccaccgtgagattcaggccttctgtataccgggcaatcatgggcgaaaatggccggtctgagacatactccagtcaattccggtacgattaggctgaactagaagaaaccaacctcctgtgatattaccctgcttgtcaagaatagtccgaacagcctttggactgttcagaacaatcacagtgcgattcatcaatttgagctagaaaccatgagaagtttattatttctatcgttctgcacgactcacagagtacacttcgccatactccctggcccactctgtcaatctggggtaaattagctttaggtggccgagttgaggacgggcaaaacatactgcattggaagccacatcttcgtcatgagatgagcatttccgagaacaggcttggtaggtggcccgggtggcaagaagttctccctggagcctagctgaaggagcttatagacggcaacagcggaacctgcagcagcagccacgatcacgggatccacgttcgcaacagacgggaggtcgacggacagcattgcgtgaataaagctcgagcggtgcaaaaggtgtatggtcccttcaccaatgtcgagctccccgggctcttaaagtggtgggggccgaactgcatggtggagaatcttgagtctggatgagcccacacgactgtttgagccaggttacgatcggggttgaactccatcctgaattgaactagttttcagaagatcctaatcactggttcgccccaatatcgccaaggggaacgaggagcattgtcaaatctacccgagcttcaagcagacatcgacgttcgaaggaaagaaggggaaaattaatgatggtccctcttggcaatatccgagatccgaaaccccttccgtttcctggaaggtgcacgttgctccaagtcgatacttgactgaaagttcttcagctccctagtcatcggacgtctgaactttgcagggtgcgtttcaattccgtcctcagcatgataagcgtctccgatcgtggacttgcgcagtctcaaggagctttcagctgcttttcgagttagtcgaatgtcggcactcggtcggacattattgcggtagtatgagagaaactccaacacaccactgcttatcatgcgcctggctcacaagagcactatgtgagacacgttcatctgctgtcatgattgtcaaaaggagctagaaaggcgtaaaaataatgtgacaggcttccgagtgtgccaacagctgggccctccgtatccatcattctaatcgttcacaccgttcacacccaggctcaggatgagaaagaatcgcatgaactctgagtatctcagcatctatactgtacaccccaaactatgcacatccaaaaaagagaatgcaatttccagaaatctagactataacgtcggcaatccccctatctaatagtctgcaacatcgtggatcacctgcacaactgactgactacgtggtaccatctcgcattcaaacggttttggcatcgagaccggaccctattgtgaacaactaagaattttgttctggcacgcgtcgtggggcactcacgggtacaacgacatcgtccttcattgacttggggctgttaggcaggggcttgatgtcgaatccccagatgatgttcaaagatacagtgcgctatcagaacatcagctatcagttgtcactgaggtcacgagcgtagcataccttgaaaatttcagccatcttgagtccaggacagagcctgcgcccagcgccgaaagtgaaggtatgacggtagccagtcaggtcaacgcttggttttgtgccaaattcagactccatgtaccgttcggggcggaaatcgtctggggcctcgaaaacatctgcaaaaaggcttcagtatggcttcacaatcagcaatacaagaggtcactcacttgggtctcgttggatgccatctatcacaaattcagaatcagcatgtcagaaaagagacagaagggtagcactcacaaaggttcatcacgatgacggtacccttcgggatgaagtagccattgtactaaggtattgtcagctccatcacctatagcgaggaaaagggtcaattacttcgaaatcctctgtcgagtaatgaggcggtacgatgggactcggaggccagatgcgagttacctgcgaggatggaatctgttaacccttcatacatcacgtcaagtcaatccacaaacctctctgacgacgcaattgaagtatttcatcttcaatgcatcttgataagttggcaaacgcgagtcgtattcatcgcccatgacctccttcagctcatcacgaatcttctgctggcattcggggtgcatcgtcatcatgagcacgaagacacgagtgaacatagcgagggtatcagttcctccgtcaatcatgacgcctccgtgatagctgcagaatgtacgtcagtttccattcaacatagcggtgatgggaaaggagagactaacgcaataagatccctatccttgaatccaaactcatccttcctctgaagaatggtctgcatgtgagacccgtcgaagacgccagcttccattctcttctcaacccttccgaggaaatcattaaagataccaagttgcttgtccttgataccttgagccatgaccctccagccggccagactatcaggaagccacttggcgagccaaggaattagagcggtgaagtgaacacctcggagacccatcatgttttcgaagtcgtgaagatattcttcgtggtagggaatgaatgggtctgaggaggtgaggacgcgttcaccataagcgatagcaacaatactggacatgctggtgcggacgagatgcctaaagaattcctgtgaaagcctctgtcaaaacagtcgatagtgctaatcgaaatcccttaccttgggctcagccaacagctccttcatcagcacgatggtctccgtctcaatgttctctgcatatcgatcaatactgtcgttgctaatgagcaacttaaaggccttgtggttgattcggaattcgtcggattctgaaacagagtcagtcagataaaggacccacgaacaaagagcttgtcgtactgtaggaggcgataggaaggaaacggtcgtctttgataggagcagggaggaaaccagtgggtctttcagcagtcttggcattcagcttgtcaagaatgccagtaacggaggctgagtctgttaggacgataacgttcttgaagaagatcttcaactatgtggcataaacttcatgaatattgagaaatgcggtgtatcgtcaatactaacgctgtatattcctccatattcttgtgcccatcggctaagcctagaaaacgtggttaggtacgtcaatgaaggagtagctttgggcaacctactgaaggtgcatgtcgtccattgctggcatctggtggagattacccaacaccggcttcgtaggtggcccaggaggtaacgtcttctccctcgaccccatacgaagcagcttgtagaccaagtagcatgccaaagggatggccacaggtgcgatcatgttgctgtcaagcagagcagccttgacttcagaaagattcatagtcgtccgaaagtctagaccctgtggaagttgggcccattccttgagttgactgatatccatggttcagcgcacttggcaagatcttgaagttcccgggttacacgccgttatcatgctgccctatacttatgagagctcaataaggttcggtccaaaacgccaatggtttgcccgtcagaatcaacacggtttgctaaccaaagggtcctgcctaatggagagtgccaatttttgatccaactctgcgccgccatcaaatgagccctcgcttcctcgaaagcatgggttttggcattttgcctccggtggcgacggttctgcttatccatgggccgccgggagatttacttggcttggctcggttagattacttggctggttcattgttatccatcagagcggtagactgacagtggcaagaattaacagccagaacaatggtctttccacggcggcttgttccatgggagaaaggcttggagcggtgtggtacgtatcttcaccataatttcatagtccacgcgatcccattcaggaaaaatgaagcacttgggtcgtaaatctccgctctctacgtgttcctgcgaagtcataaggagcgggggaacccctgtactgaaaccaagcaatacgcagcagtgaccatggaaggcaaggtcgtgctccattgttttagtcattatatgaaaatcctgctaaccatctgagtcacatagatcgcaatcgttacaggcgcatccaatggcattggactcgccaccgtcaatctcctcctcgcagcaggagcgtctgtctttggcgtagacctcgctctagcaccgccctcggtgacctccggaaaattcaaattcctacaactcaacatctgcgacaaggatgcacccgccaggattgtgtccggctccaaggacgcctttggaagcgagagaatcgacgccctcttgaacgtcgctggtatctcggactacttccagaccgcgttgaccttcgaagacgatgtatgggacagagtcatcgatgtcaacctggctgcacaagtgaggttgatgagagaggtattgaaggttatgaaggtccagaagtcaggtagtatcgtgaacgtagtcagcaagctggccctcagcggtgcttgtggaggtgtcgcatacgttgcgagtaaacatgccttggtaagaggatgtcccgctgctagcatcgtacttgctaatgcaagcaatcggcttctgtagcttggtgtgacaaagaacaccgcgtggatgttcaaggacgatggtattcgatgcaatgccgtggcgcctggctcgaccgacaccaacattcgaaacacgacagacccgaccaaaatagattacgatgcattctctcgagccatgtgagtatcttccgtggattttcgggatgtcgttcgttctctgatcaaagaccttgggataaggcctgttatcggcgtacactgcaacttgcagaccggcgagggtatgatgagccctgaacctgcagcccaagcgatcttcttcttagcttcagacttgagtaacgggacaaatggcgtcgttattccggtcgataacgggtggagtgtcatttaggtcgccgtatattataggtacacaggccagcattaggcgacttcgaattttatgtaattagccaatgttacctctgcgaaagaatgagttcttgcgtagatggatgagcctctcaagacgagcaaggaaacatacccttcttttcgtcgtcgctatctcgccttcatcatcggcataccttttccctaccatatccacatccatttcatctccggatctcgacctaatcatcccaaccagactcccaagccctgacctatccacaacacgctcagaatcatcagctctccaatcgacctcctcaagcatcatctccgggacatattccatatccataggtgccttcgttacgccatatcttgctcccaacaccccatgtgtgagaggggaacgtaggcgatagatggtgcgtataaagtacgcgtattcttgggagtgtccgtcatttgacgaagagcgtaggatcccaggttcaatttcagccacgtaggtgatatttgagtaaagcccgagggaggtatagtaccagatcctttcaacaggagtgggtaactggaaaggaagaagttgatacttgacttcgccgagcacgatttgctgggtttgggccctctagaacattctggctgtcagaactccgagtcaaggttggttggacctgagagagatgggcataccgaaacagcgattataacatccttccctccaacgcctccttcagatgaagtccaagcgaaagccatggtggttgtcggaggagatgcccaggcaaatgccatgatcgctcatggcaagaagttgatgttccgagcctgtcaagcattgttgagttttgatgattgcctaaagggaaaggagtaagcagagaggcgcccgtagaagcgcacgagttggtgtaggacgttggtccttcctcgaggtgctgtccgtacgatcaagcaaaccaggaaacaaagaaaatgttcccaagtgacaacatgatcaagagatattgatattgcggtacgaaaacatgttatgagtccccgtactcttacaccttcgcctcctctctctttagccaccaggatgaccgctgtggaagggtatcctaccctcctcacgaaattccgaaaccaggtcgttccgaccttcaagatggtttcttgttcattctccgtcaagattccggagctcgcttgtaggctggtgcgggactgatcacgaagatcagtatcttggtggacgtaaggttgagatctactcctaggacgcgtaaaaggcactgttggggtcacagtgcgcctacgcgagtggaatccccccgagtcagtgccaccttggtgctgatcagagtgtgaatgccttatccggtcattttcctgcctttttttgtagaagtacattttgtctcgtgatcatgtattctctcgcgcgcatcgcgcgtcttgaaataattaggttcttgacacctcaccaccacgatctaagtattgaggatcacacggatcctcaccctcagcacgctaagtatttcattcagttttcattctttttaaagtctacattttcgtggttgtatcgagaagcctacggcttctcttactacgccacgtttgcgactgatctctcagttctccccacttctctcgtatccaggttcccggaccgagtgcggttgattcggcaccccgcggctgcaagggattttcttcctcaccgccttacccttgttctgccccttgtctatatatcttcggtcaacttgagttcaccggaggcaatctgagcggggacagtcgcgtagaattcctctgcatatttgtgttccaaggagctgacgatgaatccggacatggtaatctctttgccgacaatcaacataggattctaggggggggcagtctaaggtcaggaagtcaagacgctatgcaaggaggacacaccttaataggcgttccgtcgccattatagcccgaaatcattccacattcctggatgtgaccgccgttagcttagttcaaatgtgcgaggacttacttacgatgaaacgcgcctttcggccggcagcgtcgagagcggcttcgagcgtttctccgccaacattgtcccagtatctatagtcaacaggtatcgtaaactcgtgtttcttccatgcacagagagagacgtacacatcaatgggcccctcctgagccagaactccagcggttttggtcgtcttataattgaaggcaacgtcagcaccaatggacttcatgaactcaactttggcctccgagccagtggatgcgatgacttttagcccatcgcgcatggctagctggatgaccatgttgtatcgacaggcgggtgagataaagaagccttgacaagggaggacacccacgagccaacggggcctgctcctgcagtcacgaatgcggtttcccccttcaaaatcgttagacggttccgtaagctggaaatcattttgcaagccttctttgcttttgaaaactccttccatgcgtaaacagccgttttaccttgcaagatttgttagttattgacaaagttcaagtgcaaacagaaatagttacctggcattcccgcagctccaatgtaagtcgaccaagacaaactttccttgttctcaagaactttgtagccatcagaagatgcgatgacgttgtattcctgatgctccagtatcttacgattagacatgttcttagaccttggttagatagagaaacggcagcatacggaagacaccatatacatgatctccagctttgacgttctcattctcagagcgaactactccgtcaccatggctatacaatctgaaggtgctagtgttagaattatcacaacatccggaaaacaaaggcaaaaatcacggtttgccgacagggaagggtggctagacccaataaaccagtgagaattatgacacctcaaaaacctatcacttaccgaatagctggacttctcaggtgcgcgcatttttcctcgcaggtaggaatcaatggacaggaccaaggtctttaccaggaaccctccattgagcggcacatgatctgtgtcgatggtctgcgactcgtcgtatacgatcgtctctccgggtacaggatatcctgtggtggaaatacttgtgagaaaaatgggtttgcaaagtcgaaccaaaaccaaacctgttgggaccttattgaacttggcgcttccgtttctaatcactggcatgtttgaaggcagtggctttgtgagaggtcaattcaggctccttataacattttcaaggattgagggctcagatttataagaaccataacgccatcggcctgcttgccatatggcactcgcgaataatgacctcatgacttccagttgcaccggtaagagtttcgaagcaatcatatggcaggcgctgcgaaagccatttgtactggaggtctctttcaagcgataatgttcaagtcaaagagcatctttccgacctcaaagcgtgatgtgaccgctgtttgacagcaccgttaaagccaaacaacgcgtacgcgtcaatttctgaagcctgtgtgtaaacagcatacgctcccaaacaggaccgggttgggtttggggttgagcaaagccttctccgcgcacgcatcaaacagaaaagaaaagaatttagattgggaaagaaataacaaccaaatacaaactacaatggcctcaataatcaagagaccaaggagcaacaggtttggccaactcaggatttagttgggtaaagtggcaacatatcggcccgagaagagcccttcttccttggccttgatttctattgaaaacgagggatgagcaatgagtagcagatgccacaagatagaaactcacgcagggccaaatgtttcgagttgaaacgacatatagcaaagttgcctgtagtgatgaatgtcagtattagcaacatttcgaaaacggcatttatcaagctcaccaactgcattccacagattatcgatcccggaatcggcccaaacgccgttcaactcgccctccacggtattaccccagaatggtttcagctgggctgtgagttcctctcccaagatttctctattcacagtgcccgtagtaccgagtctgagtggaatcaatcagaggatgaaacagcaagggtataaagtaagagaacctacccagtcgcaaaaagtataacttccgcttgaagctcagagccgtccgcgaacttgaggccagtcggagtgatacgttcaatcgagcttccagatttgagcttgatcttgccatctgcgatcaattgactagcaccagcatctgcaggcatgtctaatcaataactctgtaaacgaaatgaggaggggaggacgtaccaaggtggaatccaccgattctttcgcgaacactgaccaatgggccagcaccattcataccatcattgaggccaaatccgaccttgcgtaggccatccaagagctctctgtcggcgtgtattatcaacacagagtcattgaagaatttgtggagacttacttgtccatttcggcaataactttgacagcgcgttgagcaagccttgctcccacagcgaacgggaaggcattcaacagcctatctgcgatgtctgtgggcggagcattctcactgtacagagctgtaatcgcagtttaattgagttcagccgggaaagcagagctgaaatacctccaagcatgacttttctagaattcgccgttgtcatgatattggtcgagctcctttgatacatcgtcacatctaccaattataaaactttgaataaggatgcagaacaggtaagaccaaactacttaccgacaccgctccaatagtagtcttctgcaatatcatgagcagaggaacctgcaccgacaatgaccaccttctttccaagatggtctgttgctctgtcgtgctggatagaatggaggatttggcctttgaatgtttcctggtgtccgtatgagcgaaggtcagaggaagttgaaaagacttggtttcttaccattccagggatggaggggagctccgccttaccacttcctgctcctgtcgcgaaaaccaagtaggacatattcaaagtgattggtgattgatctccacgcttgattttgatggtccattggccagttgcttcctcctttctggcgctttcaacgatggaagaagtccaaacattgagatcaagagctgctgcgtaactttcgagccattgtccaagctgatggagctgtaaggtcccagattcacaaaaggaaaccatctcaccttcttggcaggagtgaagagtggccaagttgaagggaaactaaattaggtgagtcagtgcttgcaccagtcaacgaggtaatgaacatacggcatgtatggcatgtggtcaaaatctgtaacgaatgtgagagatgggcaacgagagtggagtttggccttacaaatgggatcgtgtagacagagcgcatcgtagcgcgtacgccagctatcaccaattcgggcattcttctcgataatcagggaaggaacgcccaaagccttgagccttgcagcaacgcataagccgctttggcctcctccaacaatcaggactttaggttgtgtgccctcgagttcgacttccttcctcctcttctcttcccatttgccatgccagggcgaagagtctcggagaccattgatctgctcggggaatcccttcaagtcctcgagattggtgaagacgcaatatcccttccatccatcggtagcagtggggaccagacgaataatgccagaggcaacgccaacatcggtttcgaagtcgaagaagaacgagatgaaggtgaagtcggggaaggggctttgtaggcccaagtagtggtcttcacgcagcttgaatgacttcggcttgacagccttgagcctatcttcgaggaactccgtgacctttgggagcccgatgaatgtacgaaagtcccacgtcaaggcgaggagatctcgccagaaggaatccgtgatgaagagagatgcaacttgtttggcatcaccggcctcggctgcagaagcaaaggcagcaaaccaatcctgggcgactttctttacatcaatgtctgctggaacgggcgctgcacccaagcggtcaagggtggccagaggaacactaagaagttggtcgagttgttcgggagtaatcgacatggtagaaatggctgatatcgtttggggaaagtcggtgcgttctacataccgattttaaatgaattgacgttcatagtggctgtaagtcgagacacggatatctcggtgggattcgtaagaacgggtcacccggtcgctccgcagccgatagaacaagttactacagggttccaagaggtcatctaagggcgtcgggtcacaacccgaaggcgtccattgatctgacattacggaaagccagccggctccgtcgcgccaggactcttgcttcgcgagaacgttggaagtgtgatcgacaacgtgggcacttgtcgacaacgaataggatggactggaaggcgtgtgtaatcatggtgaatatcccgagttgaagttcggttaacttccgaggggttgcctcttttggcaagtctagctgttcacagtcttcagaattggggggtcagccgaagtaaattcgtgttggcggctccaagacgggagagagcaacgggtatttacatttcgcgattagatgcatatagtgcccgctatcaagaagatacaatgaaatgcaagctactagacgttccagaggccatactatgtcattaaatggccataatttcttcatctttctcctggaagggaatcatagtggagctcgatgggtgatgcataaatctctgcgggaactcgtcagcgtttcatatagctctcaaaataaacatgacttacgaggggcgtttggtgcctctcaagctcgtcatggtcactgacgaactccttggtcgtgtcaatcttgatggagatcgtaccggtttggcgctgatgaatgatgagtaactgtactatacttttaggttatgacaacttaccctggaggtgatacttgcagccttgggatagcgagaaagggacaggttgtcacttgtgctctggataccatcgctggagccacggatcattttgcgggcgttgagtgtggcaagaagagagttggtgtacactgtgccaggttaatttgagttcaggaagcaggatatctggaagcatgactcacgacgaccaatgcagaagaaaaacgcgatgtagagaaaggtttgtccagctacgacgatctagggagggatcgcccgtgtaaacaattgtgaagagagacgaagtcttgccacggaagattactcactgagactaacgaagcaattgcgcagagactatcatcagcgaggtatcagctttgtaacacttatcggcggtgttagcagattacgaacctcgtcagtaagcctgtgttgacggcaaataggatctggcatgaagttagtgagaatttcggtggtgcacgaaagcttaagaagcataccagcttcttaatgatggtgtcggaactatgggagcgatcgtcatgccacgtccagaagtagattgaataatacgtaccggcggaagccagttcgagagctgtacagcaatgtgcaaagtataatcgcgatgaggacatcacccgccgccgcaaaagcattgaccatgatggacagaaactaatttgtgaaatgagcgatggaatcaggtctagagagatgaggccccaccttcagttgttgcagttcggcgaatgtgtgaaggtcgaggctgaagaaataatcagtaaccaaggaagagccagacagccgagcctttagaacgcacgacaagacgctgaacgccagaatgcatcctacccgggcaagtgagtcaacccaatatcataaataaattagtgaaagcgcaccgaactcaaggagaacaaggagaatctgaaagaggggtttgcatcagaacaacacatccggaatcggtgttctaaatactgaccacaacggcggtaagaacgatgttgtgactgctcactgccgaggtattagttaaaatgacctggtgaaattgagcaaacgcacgaagccaaaccctcaaagcgagaaatctgccgatgttgggatcagaaacgcaacaggacaatggctggatccgcgtacccttggactagaaacgccgtcaggccctgtgaagaagtcagcagcggtttcggcaagtgatggaaggagcgtacattgaaaagtacctccaccttgtcgaggatgagcgattgtttcgggagctggcctgcgaacttaccagtagactcctggaagacatcgggtgacccggtaagacaattgagggaccaaaggaagtatgctatacgtaccacaccagaacattgagcgcatccgggttgaagtagttcgtaatcgtgtatgtgtacactgcataacattatcaggggggaacgtaacgtagcatacagccaacttaccagtatgagaaataagaatttggtgaatcgtatcaaatatcataacggcagcgacctgtctaatatcaggagaaaaattctccagaacaacgggaaagcgacgaaccagtgttttcagaggccacgcatcctgctggtgagtgaagtaataccaggcctgaacacaggaaactattccaagttagagtacggcatgaaagagaaggaataattaataccaccgtgaaggctgtgtagaaaagggtcagcggatgtccggaaagggataaaacaaaagctaacttcgctgaaacgacggcgccaataaacgcagctcctaagctagaaccaccgccgtcagtgccacaagaccaataaagtcgggtaagtgccactcacgttttgtccaaagcatgagacatattcaaggtagacatgaaaaacgagagagatacagggaatggtggacaattattagacaaggaagaggatttgggacgatgagtgccagacagtcggtgaatagaggtattataagttattttgctccgtgcttgaaattcttgaagatttcagtcacaattcgtcatgcaaggaagacgcttggggagcctcgaggacgtgaccacgcagtcaaggtacggaagctgagaagcaccagatgttttgccaggtcgtgaactactgcagaagctggtcatccactctcgggtctctaaactgcccgttttatcgacagacctgtttgtaacttgaactgctaacttttccctgttttccttttttttttagagcaaaagcctagcaaacagactttgagcgtccgaagcctatttagccttacaaggtcagtcagaatcgtaaacaacccagtatttcgaggattcaagaactctgcttcgacgacaatcatgaaatggtaatgaaacacagatac

SEQ ID NO: 16 - DNA, 26 bp, Artificial
  Primer cyp450-1_3
caatgacctatgggctcgagactgaa

SEQ ID NO: 17 - DNA, 27 bp, Artificial
  Primer cyp450-1_5
gttgaggtatgggaaagatgggaagtc

SEQ ID NO: 18 - DNA, 27 bp, Artificial
  Primer cyp450-1_3n
tctgagattatgacatctggcgccttt

SEQ ID NO: 19 - DNA, 23 bp, Artificial
  Primer cyp450-1_5n
gtgcccaaggcggatgcagtcgt

SEQ ID NO: 20 - DNA, 23 bp, Artificial
  Primer predP-1_3
ctggaattgggagccgaagattt

SEQ ID NO: 21 - DNA, 27 bp, Artificial
  Primer predP-1_5
gagaaccccatcctccatctgtatgat

SEQ ID NO: 22 - DNA, 26 bp, Artificial
  Primer predP-1_3n
cgtcacaggttttcggcattacctta

SEQ ID NO: 23 - DNA, 25 bp, Artificial
  Primer predP-1_5n
cgagaggaagaatgcggtgtacagt

SEQ ID NO: 24 - DNA, 26 bp, Artificial
  Primer dts_3
cccatgacgaattcgttacagagttt

SEQ ID NO: 25 - DNA, 26 bp, Artificial
  Primer dts_5
cttcgcggattcaatgactttgtaca

SEQ ID NO: 26 - DNA, 27 bp, Artificial
  Primer dts_3n
ctgatgtcaacaagtacgaatcccaaa

SEQ ID NO: 27 - DNA, 23 bp, Artificial
  Primer dts_5n
tcgggcttctggctctggagaat

SEQ ID NO: 28 - DNA, 23 bp, Artificial
  Primer ggdps_3
agtccgctctccgtcgtggttca

SEQ ID NO: 29 - DNA, 27 bp, Artificial
  Primer ggdps_5
agcttgtggacatgaggttgatgtagt

SEQ ID NO: 30 - DNA, 26 bp, Artificial
  Primer ggdps_3n
caagacgtctatgacctcggaatgaa

SEQ ID NO: 31 - DNA, 23 bp, Artificial
  Primer ggdps_5n
gagccgtacgccaagcctgagca

SEQ ID NO: 32 - DNA, 26 bp, Artificial
  Primer cyp450-2_3
ttcttagactacatccctcgcggttt

SEQ ID NO: 33 - DNA, 25 bp, Artificial
  Primer cyp450-2_5
caaccgttccaaatcattgaagcat

SEQ ID NO: 34 - DNA, 23 bp, Artificial
  Primer cyp450-2_3n
attccggggtcaggaccggatct

SEQ ID NO: 35 - DNA, 27 bp, Artificial
  Primer cyp450-2_5n
cgattcgatgtacgatatcgtggtctt

SEQ ID NO: 36 - DNA, 23 bp, Artificial
  Primer cyp450-3_3
gcgtcatgattgacggaggaact

SEQ ID NO: 37 - DNA, 24 bp, Artificial
  Primer cyp450-3_5
cagccatcttgagtccaggacaga

SEQ ID NO: 38 - DNA, 23 bp, Artificial
  Primer cyp450-3_3n
ggcgatgaatacgactcgcgttt

SEQ ID NO: 39 - DNA, 23 bp, Artificial
  Primer cyp450-3_5n
catgtaccgttcggggcggaaat

SEQ ID NO: 40 - DNA, 1572 bp, Clitopilus passeckerianus
atggctccgtcaacggaacgtgctctaccagtccttgtaatatggactgctataggcttggcctactggatagactctcagaagaagaaaaagcagcacctgccgcctgggccaaagaaacttccaattatcggcaacgtgatggacctaccggcgaaggtcgaatgggaaacctatgctcgctggggtaaagagtacaactctgatatcatacatgttagcgccatgggaacctcgatcgtaatactgaattctgccaacgccgccaatgacttgttgctgaagaggtcggcgatctactcgagcagaccacacagcacgatgcaccacgaactgtcaggctggggctttacgtgggccttaatgccttatggcgagtcatggcgggctggtcgtagaagcttcaccaagcacttcaactcttcaaaccccggtataaaccaacctcgtgagttgcgatatgtgaaacggttcctcaagcagctttacgagaagcccgacgacgttctcgatcatgtacggaacttggtcggctctacaacgctttcaatgacctatgggctcgagactgaaccttacaacgatccctatgttgacctggtagagaaagctgtccttgcagcgtctgagattatgacatctggcgcctttcttgttgacatcatccctgccatgaaacacattcctccatgggtcccagggactatcttccatcaaaaggccgccttaatgcgaggtcatgcgtactatgttcgtgaacagccattcaaagttgcccaggatatgattaaaactggtgactatgagccctcctttgtatccgacgctcttcgagatcttgagaactcggaaaaccaggatgaagatttagagcacctcaaggatgttgctggtcaagtctacattgctggtgctgatacgactgcatccgccttgggcaccttcttcctcgccatggtctgtttccccgaagtacagaaaaaagcacaacgagaattggatagtgttctcaatggaaggatgcccgagcacgtcgacttcccatctttcccatacctcaacgctgtgatcaaagaagtctaccgctggagacctgtgactcctatgggcgtacctcatcaaaccatctcagatgacgtttacagggactaccacatccctaagggatccatcgtgttcgccaaccaatgggcaatgtccaacgacgagaccgattacccccagccggacgaattccagcctgagcgatacttgaccgaagatggcaagcctaataaggcggttagagacccctttgatattgcgttcggcttcggtagaagaatttgcgctggtcgttaccttgctcattccaccatcaccttggctgcagcctctgttctgtcgctatttgatctcttaaaagcagttgacgaaaatggcaaggaaatcgagcctactagagagtatcatcaggctatgatttcacgtccacttgatttcccgtgtcgtatcaagccaaggagcaaggaagccgaggaggttatccgtgcttgcccgttaacgttcaccaagccaactcttggtgtctag

SEQ ID NO: 41 - protein, 523 aa, Clitopilus passeckerianus
MAPSTERALPVLVIWTAIGLAYWIDSQKKKKQHLPPGPKKLPIIGNVMDLPAKVEWETYARWGKEYNSDIIHVSAMGTSIVILNSANAANDLLLKRSAIYSSRPHSTMHHELSGWGFTWALMPYGESWRAGRRSFTKHFNSSNPGINQPRELRYVKRFLKQLYEKPDDVLDHVRNLVGSTTLSMTYGLETEPYNDPYVDLVEKAVLAASEIMTSGAFLVDIIPAMKHIPPWVPGTIFHQKAALMRGHAYYVREQPFKVAQDMIKTGDYEPSFVSDALRDLENSENQDEDLEHLKDVAGQVYIAGADTTASALGTFFLAMVCFPEVQKKAQRELDSVLNGRMPEHVDFPSFPYLNAVIKEVYRWRPVTPMGVPHQTISDDVYRDYHIPKGSIVFANQWAMSNDETDYPQPDEFQPERYLTEDGKPNKAVRDPFDIAFGFGRRICAGRYLAHSTITLAAASVLSLFDLLKAVDENGKEIEPTREYHQAMISRPLDFPCRIKPRSKEAEEVIRACPLTFTKPTLGV

SEQ ID NO: 42 - DNA, 1134 bp, Clitopilus passeckerianus
atgaagcccttctcgccggaacttctggttctatctttcattctattggtactctcttgtgccattcggcctgctagaggacgatgggttctctgggttattattgttgggcttaacacctacctcaccatgaccacgaccggcgattcgaccttggattatgacattgccaacaacctcttcgttattaccctcacggccacggattatattctcttgacggacgtccagagagaattgcaattccgcaaccagaaaggtgtcgagcaagcctcgttgcttgaacgcatcaagtgggcgacatggctggtgcaaagtcggcgtggcgttggctggaattgggagccgaagattttcgtccacaggttcaccccaaagacttcacgcctttcatttctcctccagcaactcgtcacaggttttcggcattaccttatttgtgatctggtctcgctgtatagccgaagcccagtcgcattcatcgaacctcttgcttctcgccctttgatctggcggtgtgcagatattgccgcatggctcctgttcacgacaaaccaagtatcaattcttcttacggcattgagtgtcatgcaagttctctcgggctactcagaaccacaggactgggtccccgtgtttggccgctggagagatgcttctaccgttaggcggttctggggtcggtcctggcatcaattagttcgcagatgcctatcagccccaggaaaacatctttcgacgaagatgctgggcttgaagtctggctctaacccggcgctttacgtacaactgtacaccgcattcttcctctcgggagttttgcatgcgattggggatttcaaggttcacgcagattggcacaaggccgggactatggagttcttctgcgtccaagcggcgatcatacagatggaggatggggttctctgggtcggaaggaagctcggtattaaaccggcctggtactggaaggcccttggacatctttggactgtcgcatggttcgtctatagctgtccgaattggctgggggcgactgtctcaggaaggggcaaggcttcgatgtcgttggacagtagtgtcattcttggtctgtaccggggagaatggaatcctcctcgtgtagtacagtag

SEQ ID NO: 43 - protein, 377 aa, Clitopilus passeckerianus
MKPFSPELLVLSFILLVLSCAIRPARGRWVLWVIIVGLNTYLTMTTTGDSTLDYDIANNLFVITLTATDYILLTDVQRELQFRNQKGVEQASLLERIKWATWLVQSRRGVGWNWEPKIFVHRFTPKTSRLSFLLQQLVTGFRHYLICDLVSLYSRSPVAFIEPLASRPLIWRCADIAAWLLFTTNQVSILLTALSVMQVLSGYSEPQDWVPVFGRWRDASTVRRFWGRSWHQLVRRCLSAPGKHLSTKMLGLKSGSNPALYVQLYTAFFLSGVLHAIGDFKVHADWHKAGTMEFFCVQAAIIQMEDGVLWVGRKLGIKPAWYWKALGHLWTVAWFVYSCPNWLGATVSGRGKASMSLDSSVILGLYRGEWNPPRVVQ

SEQ ID NO: 44 - DNA, 1053 bp, Clitopilus passeckerianus
atgagaatacctaacgtctttctctcttacctgcgacaagtcgccgtcgacggcactctgtcatcttgttctggagtgaaatcacgaaagccggtcattgcctatggctttgacgactcacaagactctctcgtcgatgagaatgacgaaaaaatattggagccctttggctactatcgtcatcttttgaaaggcaagagcgccaggacagtgttgatgcactgcttcaacgcgttccttggactgcccgaagattgggtcattggcgtaacaaaggccattgaagaccttcataatgcatccctactaattgatgacatcgaagacgagtccgctctccgtcgtggttcaccagccgcccacatgaagtacgggattgccctgaccatgaacgcggggaatcttgtctacttcacggtccttcaagacgtctatgacctcggaatgaagacaggcggcactcaggtcgccaacgcaatggctcgcatctacactgaagagatgattgagctccaccgtggtcaaggcattgaaatctggtggcgtgaccagcggtcccctccttccgtcgatcaatacattcacatgctcgagcagaaaaccggcggcctgctcaggcttggcgtacggctcttgcaatgccatcccggtgtcaataacagggccgacctctccgacattgcgctccgtattggtgtctactaccaacttcgcgacgactacatcaacctcatgtccacaagctaccatgacgagcgtggattcgctgaggacataaccgaaggaaagtacactttcccgatgttacactcactcaagaggtcacctgattctggactgcgtgaaatcttggaccttaaaccggcagacattgccctgaagaagaaagctatcgctatcatgcaagatactggatcgcttgttgcaacccggaaccttctcggtgcagttaagaatgatctcagtggattggttgctgaacagcgtggagacgactacgctatgagcgcgggtcttgaacgattcttggaaaagttgtacatcgcagagtag

SEQ ID NO: 45 - protein, 350 aa, Clitopilus passeckerianus
MRIPNVFLSYLRQVAVDGTLSSCSGVKSRKPVIAYGFDDSQDSLVDENDEKILEPFGYYRHLLKGKSARTVLMHCFNAFLGLPEDWVIGVTKAIEDLHNASLLIDDIEDESALRRGSPAAHMKYGIALTMNAGNLVYFTVLQDVYDLGMKTGGTQVANAMARIYTEEMIELHRGQGIEIWWRDQRSPPSVDQYIHMLEQKTGGLLRLGVRLLQCHPGVNNRADLSDIALRIGVYYQLRDDYINLMSTSYHDERGFAEDITEGKYTFPMLHSLKRSPDSGLREILDLKPADIALKKKAIAIMQDTGSLVATRNLLGAVKNDLSGLVAEQRGDDYAMSAGLERFLEKLYIAE

SEQ ID NO: 46 - DNA, 1572 bp, Clitopilus passeckerianus
atgctgtccgtcgacctcccgtctgttgcgaacgtggatcccgtgatcgtggctgctgctgcaggttccgctgttgccgtctataagctccttcagctaggctccagggagaacttcttgccacccgggccacctaccaagcctgttctcggaaatgctcatctcatgacgaagatgtggcttccaatgcaattgacagagtgggccagggagtatggcgaagtgtactctctcaaattgatgaatcgcactgtgattgttctgaacagtccaaaggctgttcggactattcttgacaagcagggtaatatcacaggagaccggccattttcgcccatgattgcccggtatacagaaggcctgaatctcacggtggaaagcatggacacttccgtatggaagactggtcgcaaaggtatccacaattacctaacgccaagtgccttgagtggctacataccgcgacaagaagaggaatctgtgaacctcatgcacgatctattgatggacgctcctaatcggccgatccatattaggcgtgctatgatgtcgctactcctgcacattgtgtatggccagccacgttgcgaaagttactatggcacgattatcgagaatgcatacgaagctgccaccagaattggtcaaatcgctcacaacggtgcagcggtcgacgctttccccttcttagactacatccctcgcggtttccccggggccggctggaagaccattgtggatgaattcaaggatttccgtaatggtgtctacaattctctcttggaaggtgccaagaaggcgatggattccggggtcaggaccggatcttttgcagagtccgtgattgaccatccggatggtcgtagctggcttgagttatcaaaccttagcggtggtttcttggacgccggcgcgaagaccacgatatcgtacatcgaatcgtgtattcttgctcttatcgcccacccggactgccagcgcaagatacaggacgagctggacaatgttttggggaccgaaaccatgccatgcttcaatgatttggaacggttgccttatctcaaggcgttcctacaggaggtccttcggcttcggccagtcggccctgtagcccttccccacgtctcgcgggagagcttgtcttatggcggttacgtactgccagagggaagtatgatcttcatgaacatctggggaatgggccatgaccccgagctcttcgacgaacctgaggccttcaagcctgaacgctatttcttgtcgccaaacggcacgaagccaggcttatctgaagacgtcaatcccgatttcctgttcggtgctggacgtagagtctgcccaggcgataagctggcaaaacgatcaactggtctcttcatcatgaggctctgttgggcattcaatttttacccagattcttcaaacaaggacactgtgaagaatatgaacatggaggactgttacgacaagtcggtttctcttgagactcttccacttccgttcgcatgcaaaattgaacctcgagataagatgaaggaagacttgattaaggaagcgttcgctgcgttgtag

SEQ ID NO: 47 - protein, 523 aa, Clitopilus passeckerianus
MLSVDLPSVANVDPVIVAAAAGSAVAVYKLLQLGSRENFLPPGPPTKPVLGNAHLMTKMWLPMQLTEWAREYGEVYSLKLMNRTVIVLNSPKAVRTILDKQGNITGDRPFSPMIARYTEGLNLTVESMDTSVWKTGRKGIHNYLTPSALSGYIPRQEEESVNLMHDLLMDAPNRPIHIRRAMMSLLLHIVYGQPRCESYYGTIIENAYEAATRIGQIAHNGAAVDAFPFLDYIPRGFPGAGWKTIVDEFKDFRNGVYNSLLEGAKKAMDSGVRTGSFAESVIDHPDGRSWLELSNLSGGFLDAGAKTTISYIESCILALIAHPDCQRKIQDELDNVLGTETMPCFNDLERLPYLKAFLQEVLRLRPVGPVALPHVSRESLSYGGYVLPEGSMIFMNIWGMGHDPELFDEPEAFKPERYFLSPNGTKPGLSEDVNPDFLFGAGRRVCPGDKLAKRSTGLFIMRLCWAFNFYPDSSNKDTVKNMNMEDCYDKSVSLETLPLPFACKIEPRDKMKEDLIKEAFAAL

SEQ ID NO: 48 - DNA, 1641 bp, Clitopilus passeckerianus
atggatatcagtcaactcaaggaatgggcccaacttccacagggtctagactttcggacgactatgaatctttctgaagtcaaggctgctctgcttgacagcaacatgatcgcacctgtggccatccctttggcatgctacttggtctacaagctgcttcgtatggggtcgagggagaagacgttacctcctgggccacctacgaagccggtgttgggtaatctccaccagatgccagcaatggacgacatgcaccttcagcttagccgatgggcacaagaatatggaggaatatacagcttgaagatcttcttcaagaacgttatcgtcctaacagactcagcctccgttactggcattcttgacaagctgaatgccaagactgctgaaagacccactggtttcctccctgctcctatcaaagacgaccgtttccttcctatcgcctcctacaaatccgacgaattccgaatcaaccacaaggcctttaagttgctcattagcaacgacagtattgatcgatatgcagagaacattgagacggagaccatcgtgctgatgaaggagctgttggctgagcccaaggaattctttaggcatctcgtccgcaccagcatgtccagtattgttgctatcgcttatggtgaacgcgtcctcacctcctcagacccattcattccctaccacgaagaatatcttcacgacttcgaaaacatgatgggtctccgaggtgttcacttcaccgctctaattccttggctcgccaagtggcttcctgatagtctggccggctggagggtcatggctcaaggtatcaaggacaagcaacttggtatctttaatgatttcctcggaagggttgagaagagaatggaagctggcgtcttcgacgggtctcacatgcagaccattcttcagaggaaggatgagtttggattcaaggatagggatcttattgcctatcacggaggcgtcatgattgacggaggaactgataccctcgctatgttcactcgtgtcttcgtgctcatgatgacgatgcaccccgaatgccagcagaagattcgtgatgagctgaaggaggtcatgggcgatgaatacgactcgcgtttgccaacttatcaagatgcattgaagatgaaatacttcaattgcgtcgtcagagaggtaactcgcatctggcctccgagtcccatcgtaccgcctcattactcgacagaggatttcgaatacaatggctacttcatcccgaagggtaccgtcatcgtgatgaacctttatggcatccaacgagacccaaatgttttcgaggccccagacgatttccgccccgaacggtacatggagtctgaatttggcacaaaaccaagcgttgacctgactggctaccgtcataccttcactttcggcgctgggcgcaggctctgtcctggactcaagatggctgaaattttcaagcgcactgtatctttgaacatcatctggggattcgacatcaagcccctgcctaacagccccaagtcaatgaaggacgatgtcgttgtacccggtccggtctcgatgccaaaaccgtttgaatgcgagatggtaccacgtagtcagtcagttgtgcaggtgatccacgatgttgcagactattag

SEQ ID NO: 49 - protein, 546 aa, Clitopilus passeckerianus
MDISQLKEWAQLPQGLDFRTTMNLSEVKAALLDSNMIAPVAIPLACYLVYKLLRMGSREKTLPPGPPTKPVLGNLHQMPAMDDMHLQLSRWAQEYGGIYSLKIFFKNVIVLTDSASVTGILDKLNAKTAERPTGFLPAPIKDDRFLPIASYKSDEFRINHKAFKLLISNDSIDRYAENIETETIVLMKELLAEPKEFFRHLVRTSMSSIVAIAYGERVLTSSDPFIPYHEEYLHDFENMMGLRGVHFTALIPWLAKWLPDSLAGWRVMAQGIKDKQLGIFNDFLGRVEKRMEAGVFDGSHMQTILQRKDEFGFKDRDLIAYHGGVMIDGGTDTLAMFTRVFVLMMTMHPECQQKIRDELKEVMGDEYDSRLPTYQDALKMKYFNCVVREVTRIWPPSPIVPPHYSTEDFEYNGYFIPKGTVIVMNLYGIQRDPNVFEAPDDFRPERYMESEFGTKPSVDLTGYRHTFTFGAGRRLCPGLKMAEIFKRTVSLNIIWGFDIKPLPNSPKSMKDDVVVPGPVSMPKPFECEMVPRSQSVVQVIHDVADY

SEQ ID NO: 50 - DNA, 24 bp, Artificial
  Primer Cp_act_U1
tgatggtcaagttatcacgattgg

SEQ ID NO: 51 - DNA, 26 bp, Artificial
  Primer Cp_act_L1
gagttgtaagtggtttcgtgaatacc

SEQ ID NO: 52 - DNA, 20 bp, Artificial
  Primer Cp_cyp450-1_U1
tcggctctacaacgctttca

SEQ ID NO: 53 - DNA, 23 bp, Artificial
  Primer Cp_cyp450-1_L1
tgtcataatctcagacgctgcaa

SEQ ID NO: 54 - DNA, 23 bp, Artificial
  Cp_predP-1_U1
aagattttcgtccacaggttcac

SEQ ID NO: 55 - DNA, 25 bp, Artificial
  Primer Cp_predP-1_L1
tacagcgagaccagatcacaaataa

SEQ ID NO: 56 - DNA, 25 bp, Artificial
  Primer Cp_dts_U1
gttacagagtttgaggcacctacct

SEQ ID NO: 57 - DNA, 20 bp, Artificial
  Primer Cp_dts_L1
cgtggaggagcgacataagg

SEQ ID NO: 58 - DNA, 20 bp, Artificial
  Primer Cp_ggdps_U1
gacatcgaagacgagtccgc

SEQ ID NO: 59 - DNA, 24 bp, Artificial
  Primer Cp_ggdps_L1
ttgaaggaccgtgaagtagacaag

SEQ ID NO: 60 - DNA, 19 bp, Artificial
  Primer Cp_cyp450-2_U1
tacatccctcgcggtttcc

SEQ ID NO: 61 - DNA, 13 bp, Artificial
  Primer Cp_cyp450-2_L1
ggtcttccagccg

SEQ ID NO: 62 - DNA, 22 bp, Artificial
  Primer Cp_cyp450-3_U1
gtcatgattgacggaggaactg

SEQ ID NO: 63 - DNA, 23 bp, Artificial
  Primer Cp_cyp450-3_L1
tccttcagctcatcacgaatctt

SEQ ID NO: 64 - DNA, 400 bp, Artificial
  P2453 Hairpin; subsequence of the diterpene synthase gene (forward sequence)
tcgccctcgtcttcgccctttgtcttcttggtcatcagatcaatgaagaacgaggctctcgcgatttggtggacgttttcccctccccagtcctgaagtacttgttcaacgactgtgtcatgcactttggtacattctcaaggctcgccaacgaccttcacagtatctcccgcgacttcaacgaagtcaatctcaactccatcatgttctccgaattcaccggaccaaagtctggtaccgatacagagaaggctcgtgaagctgctctgcttgaattgaccaaattcgaacgcaaggctaccgacgatggtttcgagtacttggtccagcaactcactccacatgtcggggccaaacgcgcacgggattatatcaatataatccgcgtcacctacctgca

SEQ ID NO: 65 - DNA, 240 bp, Artificial
  P2453 Hairpin; spacer containing Intron 1 of Cutinase gene from Magnaporthe grisea
ctcgaggtacgtacaagcttgctggaggatacaggtgagcgtgagcctttcttcttgcctctctttgttttttttttgttctttttgccgaatagtgtacccactggagatttgttggccatgcaaataaatggaagggactgacaagattgtgaaattgttcaaaacacacagcacacagccagggaacggcagatcttcgcatgctaaggcctcccagcccatagtcttcttctgcat

SEQ ID NO: 66 - DNA, 400 bp, Artificial
  P2453 Hairpin; subsequence of the diterpene synthase gene (reversed complement)
tgcaggtaggtgacgcggattatattgatataatcccgtgcgcgtttggccccgacatgtggagtgagttgctggaccaagtactcgaaaccatcgtcggtagccttgcgttcgaatttggtcaattcaagcagagcagcttcacgagccttctctgtatcggtaccagactttggtccggtgaattcggagaacatgatggagttgagattgacttcgttgaagtcgcgggagatactgtgaaggtcgttggcgagccttgagaatgtaccaaagtgcatgacacagtcgttgaacaagtacttcaggactggggaggggaaaacgtccaccaaatcgcgagagcctcgttcttcattgatctgatgaccaagaagacaaagggcgaagacgagggcga

SEQ ID NO: 67 - DNA, 2126 bp, Artificial
  P2453 ;  promoter sequence
gcacgcaattaagtatgttcgtcctgcggtagaaggttttcaagtagacgtacttcgtaggatcatccgggtattttgacctcaagtcttggttcttgttcacggcccgttcaaatttcagaagtgttctccgtatggagggagctgaaagttcttcagcctgcgaagggtgagcatccaagttagttcgaggccactatacgacactcacatcttcctgcactccttccccagcagcattctcaaatatcttgaggatatccttttgctccgacgttaacccccctccgtagaaccgaccctcttcatcttcttccgcgaagtagtctgcatcaccacctggcgcgaagtctccagcatcttcatccggcacgtcttcaacgcgggcagcgcgtctttgtctgctgccctcaggcggctctccattcatttcaacatccatactgggaccagcagcagcacttccattgtcaagcttcatcttcttcaacatttcaggagtgggattatccggtagcttcctcttgttcccagtcaaaggaacttttgggaccttgagggacacgtcaaaccttcaataactttagcttagaagcagtctttactgactttgaatagactgtcgatatccattggtagtcctcagtggttggtcgaacagaatgtggcaagcaaagtagcaaacgtgtttacgtaatgtaatgaattcgttcatagccccctcaacagctcgtacacacaggacatggctcaaattcagatgtattatggtactttcaacacacagaacgccacatatgcttaccagaagcgacaacttagggagtaaaatcctgaagttcatgaaaccctcaaagtgtcaatcatcattgttcaagcacatctaagcaaggcctcacattatacagcagcgatagcgtaacgttgtctgaagtccttctaatatgcctgaaaagtttagtagggctttttgcgattcttcttcaactcctgctcgagttgcctggcctttctgtggccaatctccacaggccggatggcagtgctgtctgctttcttcagtttaatgggtcggttgccgacatatttacctgaaagtatcatcagtgagcgtagcaaaaaagaaaggtcaatgcttaccatccatctccttccatgccttcaagaaatcttcaggatcggcgaatgcaacgaaaccgtactttgcctaataatagttggtaagtcgatgttgaatggaatatgagagattgtccatttacctttccactgagccggtcacggataacacgcgctttctggaaggagacatacttgttgaaggcatttgaaaggacgtcgtcagaaacgtcgttgctaagatcgccaacaaacaaacggaaccatgctagagaacggtaatgtcatataaatggatgcaatgtaagaatcggagagaaacacacatggattccactccagcagcgtctggtcctcccaaacttttcctgctcccttcctcagaactgtggttctctttccaccctttgcaagtttgcctccagcccctccacgcttgtctatcgcagccccgggaacgtaaacactttgctgagcgagtatcccgacatcgtattcgtaagcattggcgggcacaggcgcggaatgcgaggatgaagcaaccgggaaagaggggccttgataaggtttgtagtaaggattaatatcctgcccttgcgacgtctgctgctgttggtattgctgataataattctgactataatccatctataccgacctgaatgaacgtcgtcgaagtgaaagaaaatgcggagaaacgggatgatggcagtctgcagtcaagcactgcaacaagcctgcacagacggcagtgctgctgactcagcatacgcttatgtaatcccctctgtgaacagagaatctgtgtagatcgacgagggcaacacggtcgccgtcctcaaaaccctcctccctcaaggtatgttaccgttacaaacgattgaaagccattctgtatgctgcgcgaatgtatcccagttgaattggagcgaaatctgcagtattcaggatggatgcacattctcggatttggatgtcaacgcaaaagtactgacatatcgtgatag

SEQ ID NO: 68 - DNA, 197 bp, Artificial
  P2453;  terminator sequence
tttgctacttcactctcaccttcacgcactttctttcatgtaccatgagcatatgtcgatatggatatcacaccaaaatgcattcaactatgctggccaaaaaacatgcatcacgaacgggatattatttaaccttggctgccgccaaaactatactcttgacccaagcaagcaagcctacagacttgtcgccggaa

SEQ ID NO: 69 - DNA, 620 bp, Artificial
  P2558; psubsequence of the diterpen synthase gene
gggcaaccttaaatccatatccgagaagctcctgtctagggtgtccatcgcctgcttcacgatgatcagtcgtattctccagagccagaagcccgatggctcttggggatgcgctgaagaaacctcatacgctctcattacactcgccaacgtcgcttctcttcccacttgcgacctcatccgcgaccacctgtacaaagtcattgaatccgcgaaggcatacctcacccccatcttctacgcccgccctgctgccaaaccggaggaccgtgtctggattgacaaggttacatacagcgtcgagtcattccgcgatgcctaccttgtttctgctctcaacgtacccatcccccgcttcgatccatcttccatcagcactcttcctgctatctcgcaaaccttgccaaaggaactctctaagttcttcgggcgtcttgacatgttcaagcctgctcctgaatggcgcaagcttacgtggggcattgaggccactctcatgggccccgagcttaaccgtgttccatcgtccacgttcgccaaggtagagaagggagcggcgggcaaatggttcgagttcttgccatacatgaccatcgctccaagtagcttggaaggcactc

SEQ ID NO: 70 - DNA, 23 bp, Artificial
  Primer Cp_DTS_U2
aatcgtcaagatcgccacttatg

SEQ ID NO: 71 - DNA, 27 bp, Artificial
  Primer Cp_DTS_L2
gagtaccattctgatacattccatttg

(19) United States
(12) Patent Application Publication
(10) Pub. No.: US 2013/0171701 A1
(43) Pub. Date: Jul. 4, 2013

(54) POLYPEPTIDE HAVING DITERPENE SYNTHASE ACTIVITY

(71) Applicants: Rudolf Mitterbauer, Kundl (AT); Thomas Specht, Kundl (AT)

(72) Inventors: Rudolf Mitterbauer, Kundl (AT); Thomas Specht, Kundl (AT)

(21) Appl. No.: 13/583,562

(22) PCT Filed: Mar. 9, 2011
(86) PCT No.: PCT/EP11/53571

§ 371 Date: Jan. 28, 2013

(30) Foreign Application Priority Data
Mar. 9, 2010 (EP) 10002448.8

(51) Int. Cl. C12N 9/88

(52) U.S. Cl. CPC C12N 9/88
USPC 435/127; 435/232; 536/23.2; 435/320.1; 435/254.11

(57) ABSTRACT

The present application, among others, relates to novel polypeptides having diterpene synthase activity, nucleic acid molecules encoding same, as well as to a gene cluster from Clitopilus passeckerianus which is thought to be involved in the biosynthetic pathway for producing pleuromutilin.

### FIELD OF THE INVENTION

The present application, among others, relates to novel polypeptides having diterpene synthase activity, nucleic acid molecules encoding same, as well as to a gene cluster derived from Clitopilus passeckerianus, which cluster is considered to be involved in the biosynthetic pathway for producing a diterpene, more precisely pleuromutilin.

### BACKGROUND OF THE INVENTION

Both terpenes and terpenoids are diverse and very large classes of mostly naturally-occurring organic chemicals that are derived from five-carbon isoprene units, which are assembled and then modified in numerous ways. Both terpenes and terpenoids may differ from one another in their carbon skeletons and in their functional groups. The majority of terpenes and terpenoids comprise one or more cyclic structures. Terpenoids are commonly also referred to as isoprenoids. Terpenes and terpenoids may be classified according to the number of terpene units (C5) which are part of their skeleton. Accordingly, monoterpenes are composed of two isoprene units (C10, skeleton of 10 carbon atoms), sesquiterpenes are composed of three isoprene units (C15, skeleton of 15 carbon atoms), diterpenes are composed of four isoprene units (C20, skeleton of 20 carbon atoms), and the like. Diterpenes and diterpenoids are commonly derived from geranylgeranyl pyrophosphate (GGPP).

Diterpene synthases are enzymes well-known to the skilled person, which usually catalyze a reaction using geranylgeranyl pyrophosphate (GGPP) as a substrate to form a diterpene or diterpenoid, respectively. Here, GGPP is usually transformed into a cyclic compound comprising one or more carbocycles. Accordingly, diterpene synthases are often referred to as diterpene cyclases. Diterpene synthases are commonly involved in a biosynthetic pathway for producing a diterpene or diterpenoid.

Gene clusters are commonly known as a group of neighbouring genes building a functional unit, e.g. by encoding polypeptides involved in one particular biosynthetic pathway, such as a pathway for producing a secondary metabolite. Secondary metabolites are substances that are usually produced by a certain organism under specific conditions. As opposed to primary metabolites, they are normally not essential for the organism that produces them. The broad-spectrum antibiotic pleuromutilin, a terpenoid, more precisely a diterpenoid, is one example for a fungal secondary metabolite. It was first isolated in 1951 from Pleurotus mutilus (Fr.) Sacc. and Pleurotus passeckerianus Pil. Primarily, pleuromutilin inhibits the growth of Gram-positive bacteria and of Mycoplasma. Pleuromutilin binds to the peptidyl transferase component of the 50S subunit of ribosomes and inhibits protein synthesis in bacteria. Pleuromutilin is only one member of the group of pleuromutilin antibiotics, which further comprise a number of semisynthetic derivatives including tiamulin, which has been described to be effective for the treatment of dysentery and pneumonia in swine, valnemulin and retapamulin (cf. Yao, 2007):

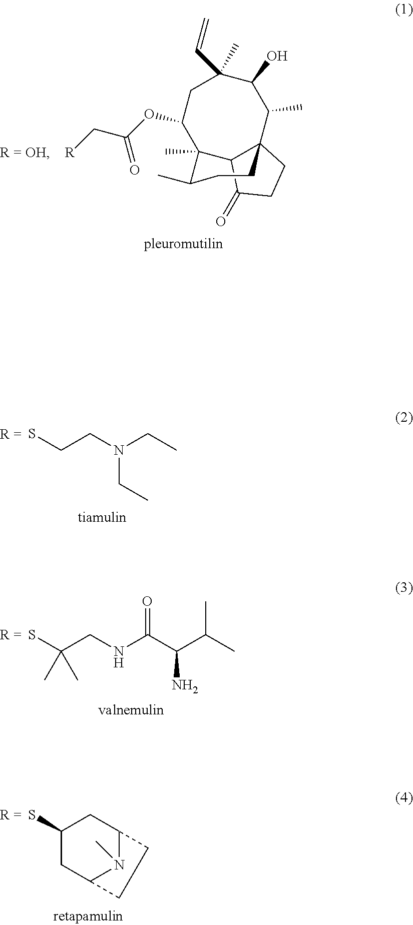

Other exemplary members of the group of pleuromutilins are azamulin and BC-3781. Further pleuromutilins are disclosed in Hunt, E., 2000, and in the references cited therein. Pleuromutilins have originally predominantly been used in veterinary medicine but they are gaining increasing interest as a human therapeutic (cf. Hu et al., 2009).

The general biosynthetic pathway for producing pleuromutilin was uncovered by isotope labeling experiments in the 1960s. An important reaction in the biosynthetic pathway for producing pleuromutilin is the reaction of geranylgeranyl pyrophosphate (GGPP), into a tricyclic pleuromutilin precursor, which reaction is thought to be catalyzed by a particular diterpene synthase (DS), a postulated enzyme commonly referred to as pleuromutilin synthase. Details of said proposed reaction (cf. Yao, 2007) are outlined in FIG. 1. In the subsequent reactions of the pathway for producing pleuromutilin, the actions of cytochrome P-450 enzymes (functions at C3 and C11) and an acyltransferase (functions at C14 hydroxyl) are considered necessary to complete formation of pleuromutilin (cf. Yao, 2007). The proposed later stages of the formation of pleuromutilin from GGPP (cf. Tsukagoshi, et al., 2007) are outlined in FIG. 2.

Clustering of the genes responsible for biosynthesis of secondary metabolites is a common feature in most microorganisms, including Streptomycetes (Ikeda at al., 2003; Oiynyk et al., 2007) and fungi (Keller et al., 2007). Efforts have been made to identify a biosynthetic gene cluster for the formation of pleuromutilin. For example, Yao (2007) describes three distinct attempts to identify diterpene synthase genes for the formation of pleuromutilin. While Yao discovered several GGPP synthase genes (ggs genes), no diterpene synthase gene could be identified.

Accordingly, there is a need in the art for identifying nucleic acids such as a gene cluster and genes encoding a diterpene synthase, as well as for identifying polypeptides encoded thereby, as the identification of the pleuromutilin gene cluster would open the path towards a rational manipulation of pleuromutilin production. Due to increasing problems with antibiotic resistance there is a particular need in the art to provide tools for producing alternative antibiotics such as pleuromutilin or precursors or variants thereof for possible use in medicinal applications.

The present inventors have succeeded in identifying a nucleic acid sequence which is contemplated to comprise a gene cluster involved in the biosynthetic pathway for producing a diterpenoid, more precisely pleuromutilin. Said nucleic acid sequence is derived from the genome of Clitopilus passeckerianus, and is envisaged to comprise at least six transcriptionally co-regulated open reading frames encoding polypeptides which are thought to be involved in pleuromutilin biosynthesis. Moreover, the present inventors have further succeeded in identifying, as part of this gene cluster, a polypeptide which is a new diterpene synthase. It appears to be the only diterpene synthase in the genome of Clitopilus passeckerianus and is thus envisaged to be the long-sought pleuromutilin synthase. Said diterpene synthase gene is in close proximity, and in fact co-regulated, with a putative geranylgeranyl diphosphate synthase gene, more than one cytochrome p450 enzyme-encoding genes and a putative acyltransferase-encoding gene, making it plausible and credible that it is involved in the biosynthetic pathway for producing pleuromutilin of Clitopilus passeckerianus. With the discovery of the biosynthetic gene cluster for pleuromutilin synthesis, the present invention is moreover believed to provide valuable tools for producing diterpenoids, particularly pleuromutilin and pleuromutilin precursors, and to provide a basis for synthesizing novel pleuromutilin antibiotics or pleuromutilin analogues, e.g. by starting from the product of the diterpene synthases disclosed herein.

### SUMMARY OF THE INVENTION

The present invention, among others, relates to a polypeptide having diterpene synthase activity as defined in the appended claims.

In another aspect, the invention relates to corresponding nucleic acid molecules as defined in the appended claims. A particular nucleic acid molecule is one which is envisaged to be a gene cluster involved in the biosynthetic pathway for producing a diterpene, more precisely pleuromutilin.

According to further aspects, the invention relates to subject-matter such as methods and uses as defined in the claims and described hereinbelow.

The aspects and particular embodiments of the invention are set forth in the claims and in the following disclosure.

### DETAILED DESCRIPTION OF THE INVENTION

### Definitions

As used herein, technical terms generally have the common meaning as understood in the art, unless defined otherwise hereinbelow.

Generally, as used herein, the term “comprising” includes the meanings “having” and “consisting of”.

Polypeptides are usually linear amino acid polymers, wherein the individual amino acids are linked to one another via peptide bonds. Polypeptides which contain a low percentage, e.g. less than 10%, 5%, 3% or even less than 1%, such as from greater than 0% to 1% of modified or non-natural amino acids are also envisaged. Preferred polypeptides, however, do not contain non-natural amino acids and modifications are only naturally occurring modifications, such as glycosylation, ubiquitination or the like. As is well known to the skilled person, a polypeptide may, for example, be modified by the phosphorylation of serine, threonine or tyrosine residue(s) by phosphorylation, or by glycosylation of e.g. asparagine residue(s) or serine residue(s). Modified polypeptides are likewise envisaged herein as comprised by polypeptides.

The term “wild type” when used herein in connection with a polypeptide refers to a naturally occurring, non-mutated form of such polypeptide.

Polypeptides of the invention particularly include non-natural polypeptides. A “non-natural polypeptide” as referred to herein does not occur as such in nature, but can be, and in particular has been, produced by laboratory manipulations, such as genetic engineering techniques or chemical coupling of other molecules to a polypeptide. Examples of modified polypeptides are polypeptides carrying in particular additions, substitutions, deletions, truncations produced by genetic engineering techniques. Preferably, a “non-natural polypeptide” is a polypeptide which is not encoded as such by the genome of a naturally occurring species, in particular a polypeptide that is not identical to any one of those polypeptides of the gene bank database as of the filing date of this application with a naturally occurring species identified as its source. In certain preferred general embodiments, the polypeptides referred to herein are non-natural polypeptides.

A “polypeptide” as used herein particularly relates to a molecule comprising more than 30, and in particular at least 35, 40, 45 or at least 50 or 100 amino acids, but not more than 10,000, in particular not more than 9,000, 8,000, 7,000, 6,000 or 5,000 amino acids.

A preferred diterpene synthase of the present invention comprises from about 500 to about 1,500 amino acids, particularly from about 800 to about 1,200 amino acids, especially from about 900 to about 1,100 amino acids, more particularly from about 900 to about 1,020 amino acids. Preferably, the molecular weight of this polypeptide is between 90 kDa and 140 kDa, particularly between 100 kDa and 130 kDa, especially between 105 kDa and 120 kDa.

An “isolated” polypeptide is meant to be a polypeptide that is present in an environment which differs from the environment in which it is naturally present or in which it was produced. Preferably, an isolated polypeptide is at least 0.01%, particularly at least 0.1%, more particularly at least 1%, more particularly at least 10%, such as at least 20%, 30%, 40%, 50%, 60%, 70%, 80%, more particularly at least 90%, such as at least 95%, 96%, 97%, 98%, 99% pure as determined according to European Pharmacopoeia 6.6, hereby incorporated by reference, by denaturing, discontinuous SDS PAGE with a 12% resolving gel and Coomassie staining. Preferably, an isolated polypeptide is not contained in an organism or a cell. In a preferred embodiment, the isolated polypeptide is not associated with any polypeptide component of its natural environment or of the production environment.

In case of polypeptides, preferably, the nature of amino acid residue changes by which the polypeptide having at least X % identity to a reference sequence differs from said reference sequence is a semi-conservative and more preferably a conservative amino acid residue exchange.

| Amino acid | Conservative exchange | Semi-conservative exchange |
|---|---|---|
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q; |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C; |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C |

Changing from A, F, H, I, L, M, P, V, W or Y to C is semi-conservative if the new cysteine remains as a free thiol. Changing from M to E, R or K is semi-conservative if the ionic tip of the new side group can reach the protein surface while the methylene groups make hydrophobic contacts. Changing from P to one of K, R, E or D is semi-conservative, if the side group is on the surface of the protein. Furthermore, the skilled person will appreciate that Glycines at sterically demanding positions should not be substituted and that P should not be introduced into parts of the protein which have an alpha-helical or a beta sheet structure.

Nucleic acid molecules are well known to the skilled person. Preferably, a “nucleic acid molecule” as used herein relates to a nucleic acid polymer consisting of nucleotide monomers, such as a DNA or RNA.

An “isolated” nucleic acid molecule is meant to be a nucleic acid molecule that is present in an environment which differs from the environment in which it is naturally present or in which it was produced. Particularly, it is separated from at least one nucleic acid molecule with which it is ordinarily associated its natural environment or the production environment, respectively. Preferably, an isolated nucleic acid molecule is not contained in an organism or a cell. In a preferred embodiment, the isolated nucleic acid molecule is not associated with any nucleic acid molecule associated with its natural environment or the production environment.

The term “wild type” when used herein in connection with a nucleic acid molecule refers to a naturally occurring, non-mutated form of such nucleic acid molecule. Nucleic acid molecules of the invention particularly include non-natural nucleic acid molecules. A “non-natural nucleic acid molecule” as referred to herein does not occur as such in nature, but can be, and in particular has been, produced by laboratory manipulations, such as genetic engineering techniques or chemical coupling of other molecules to a polypeptide. Examples of modified nucleic acid molecule are nucleic acid molecules carrying in particular additions, substitutions, deletions, truncations produced by genetic engineering techniques. Preferably, a “non-natural nucleic acid molecule” is a nucleic acid molecule which is not identical to one of those nucleic acid molecules of the gene bank database as of the filing date of this application with a naturally occurring species identified as its source. In certain preferred general embodiments, the nucleic acid molecules referred to herein are non-natural nucleic acid molecules.

A “partial sequence of a sequence x” generally refers to a sequence comprising one or more contiguous sections of the sequence x. In certain preferred embodiments, it comprises one contiguous section of the sequence x. Preferably, in case of a nucleotide sequence, the partial sequence comprises one or more, particularly one open reading frame. More preferably, the partial sequence is a sequence encoding a polypeptide, particularly a coding sequence not comprising introns (cds).

As used herein, a nucleotide sequence is said to be “of species origin” if it is contained anywhere within the genome of said species. Said nucleotide sequence may or may not be part of a gene.

The determination of corresponding positions in related sequences as well as the calculation of percentages of identity can be performed with the help of well known alignment algorithms and optionally computer programs using these algorithms. The identities in this patent application have been calculated by aligning sequences with the freeware program ClustalX (Version 1.83) with default parameters and subsequent counting of identical residues by hand. Percentage identity (PID) was then calculated by dividing the number of identities by the (entire) length of the shortest sequence. Default settings for, e.g., pairwise alignment (slow-accurate) are: gap opening parameter: 10.00; gap extension parameter 0.10; Protein weight matrix: Gonnet 250; DNA weight matrix IUS. The ClustalX program is described in detail in Thompson et al., 1997.

Accordingly, as used herein, a nucleotide or amino acid sequence is said to have “X % sequence identity” to a given sequence if an alignment with the freeware program ClustalX (Version 1.83) with default parameters, a subsequent determination of identical residues, such as by counting by hand, and a subsequent calculation of the percentage identity (PID) by dividing the number of identities by the (entire) length of the shortest sequence gives “X % sequence identity”.

A “vector” as used herein particularly relates to DNA elements that may be used for transferring and introducing foreign DNA sequence into a host. Vectors include, but are not limited to plasmids, viruses, phages, and cosmids. In a preferred embodiment, the vector is an “expression vector”. As is known to the skilled person, an expression vector is designed such that a coding sequence inserted at a particular site can be transcribed and translated into a polypeptide.

A “host” as used herein is preferably selected from a cell, tissue and non-human organism. In preferred embodiments, the host is a fungal host, more particularly a fungus from the division basidomycota, even more particularly from the order agaricales, even more particularly from the family entolomataceae. In a preferred embodiment, the host is from the genus Clitopilus and is particularly selected from the group consisting of Clitopilus scyphoides, Clitopilus prunulus, Clitopilus hobsonii, Clitopilus pseudo-pinsitus, Clitopilus pinsitus and Clitopilus passeckerianus, more particularly selected from Clitopilus pinsitus and Clitopilus passeckerianus. In another preferred embodiment, the host is from the genus Pleurotus. Preferably, the host is selected from a cell, tissue and non-human organism which is known to be capable of producing pleuromutilin, such as from the group consisting of Omphalina mutila, Clitopilus scyphoides, Clitopilus prunulus, Clitopilus hobsonii, Clitopilus pseudo-pinsitus, Clitopilus pinsitus and Clitopilus passeckerianus. Particularly, the host is Clitopilus pinsitus or Clitopilus passeckerianus.

A “cell” as used herein is not particularly limited. Preferably, said cell is one in which a vector of the invention can replicate. Preferably, said cell is one in which a coding sequence inserted in a vector is transcribed and translated into a polypeptide. Preferably, said cell is not a totipotent stem cell. In some embodiments, the tissue is a non-animal cell. In preferred embodiments, said cell is from a fungus as described above.

A “tissue” as used herein is not particularly limited. Preferably, said tissue is one in which a vector of the invention can replicate. Preferably, said tissue is one in which a coding sequence inserted in a vector is transcribed and translated into a polypeptide. In some embodiments, the tissue is a non-animal tissue. In preferred embodiments, said tissue is from a fungus as described above. A tissue may be an organ. One preferred fungal tissue is a mycelium.

A “non-human organism” as used herein is not particularly limited. Preferably, said non-human organism is one in which a vector of the invention can replicate. Preferably, said non-human organism is one in which a coding sequence inserted in a vector is transcribed and translated into a polypeptide. In preferred embodiments, the non-human organism is a non-animal organism. In preferred embodiments, the non-human organism is a fungal organism, more particularly a fungus from the division basidomycota, even more particularly from the order agaricales, even more particularly from the family entolomataceae. In a preferred embodiment, the fungal organism is a fungus from the genus Clitopilus or from the genus Pleurotus.

Both the term “wild type host” and the term “naturally occurring host” as used herein refer to a host that occurs in nature. Particularly, such wild type host is any cell, tissue or non-human organism that is or is part of a naturally occurring species that is part of database as of the filing date of this application.

The term “non-naturally occurring host” refer to a host that does not occur in nature. In preferred embodiments, said host is non-naturally occurring due to the introduction therein of e.g. a nucleic acid molecule or vector of the present invention. In preferred embodiments, said host is non-naturally occurring due to the modification therein of a nucleic acid molecule having a sequence of a nucleic acid molecule described herein.

A “corresponding naturally occurring host” of a non-naturally occurring host refers to a corresponding host that occurs in nature and does not show the non-natural feature of the corresponding non-naturally occurring host. Generally, the corresponding naturally occurring host may be a host capable or incapable of producing pleuromutilin. Generally, the corresponding naturally occurring host may be a host capable or incapable of producing a pleuromutilin precursor, particularly the compound according to formula (I). As used herein, a “host incapable of producing pleuromutilin” refers to a host which produces no detectable amounts of pleuromutilin. Accordingly, as used herein, a “host capable of producing pleuromutilin” refers to a host which produces detectable amounts of pleuromutilin in accordance with one of the above methods.

Whether or not a given host produces detectable amounts of pleuromutilin may easily be determined by extracting a homogenized sample of cells of said host with a suitable solvent and assaying for the presence or absence of pleuromutilin in said extract e.g. by a method involving HPLC (e.g. as described in Hartley et al., 2009), MS (e.g. as described in Tsukagoshi, et al., 2007) or NMR or MS (e.g. as described in Yao, 2007), preferably while also using a sample containing pleuromutilin as a positive control.

As to a fungal host, a “host incapable of producing pleuromutilin” preferably means a host showing a negative result for pleuromutilin production as determined by the “assessment of pleuromutilin production” described on page 26 of Hartley et al., 2009. As to a non-fungal host, a “host incapable of producing pleuromutilin” preferably means a host showing a negative result for pleuromutilin production when subjecting an extract of a homogenized sample of cells of said host to the HPLC analysis described in the “assessment of pleuromutilin production” on page 26 of Hartley et al., 2009.

Accordingly, a fungal “host capable of producing pleuromutilin” preferably means a host showing a positive result for pleuromutilin production as determined by the “assessment of pleuromutilin production” described on page 26 of Hartley et al., 2009, i.e. the observation of a “pleuromutilin peak”. As to a non-fungal host, a “host capable of producing pleuromutilin” preferably means a host showing a positive result for pleuromutilin production when subjecting an extract of a homogenized sample of cells of said host to the HPLC analysis described in the “assessment of pleuromutilin production” on page 26 of Hartley et al., 2009.

Non-limiting exemplary fungal strains that are capable of producing pleuromutilin are e.g. disclosed in Hartley et al., 2009. These include, but are not limited to strains of Omphalina mutila, Clitopilus hobsonii, Clitopilus pinsitus and Clitopilus passeckerianus.

A gene cluster may commonly refer to a group of genes building a functional unit. As used herein, a “gene cluster” is a nucleic acid comprising sequences encoding for polypeptides that are involved together in at least one biosynthetic pathway, preferably in one biosynthetic pathway. Particularly, said sequences are adjacent. Preferably, said sequences directly follow each other, wherein they are separated by varying amounts of non-coding DNA. Preferably, a gene cluster of the invention has a size from 10 kb to 50 kb, more preferably from 14 kb to 40 kb, even more preferably from 15 kb to 35 kb, even more preferably from 20 kb to 30 kb, particularly from 23 kb to 28 kb.

Terpenes and terpenoids are well-known to the skilled person as already described in the introduction herein. As used herein, no distinction is made between a “terpene” and a “terpenoid”. Accordingly, as used herein, no distinction is made between a “diterpene” and a “diterpenoid”. A diterpene may comprise 15 carbon atoms. Preferably, a diterpene comprises one or more cyclic structural elements. A diterpene may also comprise more or less than 15 carbon atoms. Exemplary diterpenes/diterpenoids include but are not limited to aconitine, cafestol, cembrene, kahweol, phytane, retinol, stevioside, and taxadiene. Preferred examples of diterpenes/diterpenoids include, but are not limited to pleuromutilin, tiamulin, valnemulin, retapamulin, wherein pleuromutilin is particularly preferred.

Pleuromutilin is well-known to the skilled person as a fused 5-6-8 tricyclic diterpenoid. It may be depicted as “pleuromutilin 1” in FIG. 1.

A “pleuromutilin antibiotic” as used herein refers to any antibacterial agent of the pleuromutilin family of antibiotics. Pleuromutilin antibiotics include, but are not limited to pleuromutilin, tiamulin, valnemulin, retapamulin, azamulin, BC-3781 and the ones disclosed in Hunt, E., 2000. Herein, a pleuromutilin antibiotic is preferably pleuromutilin or a pleuromutilin derivative. Pleuromutilin derivatives are not particularly limited and include any conceivable pleuromutilin derivative.

A “pleuromutilin precursor” as used herein refers to any intermediate compound of the biosynthetic pathway for producing pleuromutilin. A pleuromutilin precursor may or may not be the product of a geranylgeranyl pyrophosphate synthase and hence may or may not be geranylgeranyl pyrophosphate. Preferably, a “pleuromutilin precursor” refers to any intermediate compound of the biosynthetic pathway for producing pleuromutilin downstream of geranylgeranyl pyrophosphate. A preferred “pleuromutilin precursor” is the product of a diterpene synthase, particularly a pleuromutilin synthase, as disclosed herein. A preferred pleuromutilin precursor herein is a pleuromutilin precursor depicted in FIG. 1. An especially preferred pleuromutilin precursor herein is the compound according to formula (I) as depicted in FIG. 1.

A “pleuromutilin precursor” may also be the product of any other reaction catalyzed by a polypeptide involved in the biosynthetic pathway for producing pleuromutilin. Other preferred pleuromutilin precursors particularly include the compounds (II) and (III) depicted in FIG. 2 herein.

In addition, “diterpene synthase” and “diterpenoid synthase” are used interchangeably herein. In addition, a “diterpene synthase” may also be referred to as a “diterpene cyclase”. Diterpene synthases are well-known to the skilled person as also described hereinabove. That is, the skilled person will readily be in the position to recognize a diterpene synthase, such as by way of its homology to known diterpene synthases. To this end, the skilled person may suitably employ computational methods such as an alignment, e.g. an alignment similar to the one disclosed herein in FIG. 5. Herein, a “diterpene synthase” particularly refers to a polypeptide capable of catalyzing a conversion of geranylgeranyl pyrophosphate into a molecule containing one or more cyclic structures. Preferably, said molecule is a pleuromutilin precursor. Accordingly, in preferred embodiments, the diterpene synthase is a pleuromutilin synthase. Herein, a “pleuromutilin synthase” particularly refers to a polypeptide capable of catalyzing the conversion of geranylgeranyl pyrophosphate into a molecule containing one or more cyclic structures which molecule is a pleuromutilin precursor, particularly a pleuromutilin precursor depicted in FIG. 1, especially a compound according to formula (I).

As used herein, a “polypeptide having diterpene synthase activity” preferably refers to a polypeptide that is capable of catalyzing a conversion of geranylgeranyl pyrophosphate into a molecule containing one or more cyclic structures. Said polypeptide may or may not have further activities and diterpene synthase activity may not be the main activity of said polypeptide. Diterpene synthase activity may, for example, be detected and/or measured by incubating a sample containing the polypeptide, preferably the purified polypeptide, with GGDP in a suitable buffer and detecting the production of a product of a diterpene synthase (e.g. a diterpene molecule containing one or more cyclic structures), optionally in connection with the consumption of GGDP, by a suitable method e.g. involving MS or GC/MS. One particular exemplary enzyme assay for the activity of a diterpene synthase is disclosed in Toyomasu et al., 2000. Said assay basically involves the detection of the product of a diterpene synthase by means of GC-MS. Exemplary assays for diterpene synthase activity are also disclosed in Kawaide et al., 1997, one particular assay involving detection of GGDP consumption and product formation by employing [³H]GGDP, another one being suitable to identify metabolites by GC-MS. If necessary for a given assay, a diterpene synthase may be expressed e.g. in a fungal host or in E. coli and may optionally be purified therefrom.

As used herein, a “polypeptide having pleuromutilin synthase activity” preferably refers to a polypeptide that is capable of catalyzing the conversion of geranylgeranyl pyrophosphate into a pleuromutilin precursor, particularly into a pleuromutilin precursor depicted in FIG. 1, especially into a compound according to formula (I). Said conversion may comprise one or more steps. Said polypeptide may or may not have further activities and pleuromutilin synthase activity may not be the main activity of said polypeptide. Pleuromutilin synthase activity may, for example, be detected and/or measured by incubating a sample containing the polypeptide, preferably the purified polypeptide, with GGDP in a suitable buffer and assaying for the production of a pleuromutilin precursor, particularly a compound according to formula (I). The detection of the production of a pleuromutilin precursor, particularly of a compound depicted in FIG. 1, especially of a compound according to formula (I) will easily be achievable by the skilled person on basis of the disclosure herein including the teachings of the references disclosed herein; e.g. by a method involving MS or NMR. In preferred embodiments, a pleuromutilin synthase of the invention has at least 5%, 10% or 20%, preferably at least 30% or 40%, such as at least 50%, or at least 60%, 70%, 80%, particularly at least 90% or 95%, especially at least 100%, such as at least 125%, 150% or 175% of the activity of the pleuromutilin synthase of SEQ ID NO: 9, particularly at least 2-fold or 5-fold, at least 10-fold or 25-fold, such as from 50- to 100-fold the activity of the pleuromutilin synthase of SEQ ID NO: 9.

The terms “geranylgeranyl pyrophosphate”, which is abbreviated as GGPP, and “geranylgeranyl diphosphate”, which is abbreviated as GGDP, are used interchangeably herein.

As used herein, the “biosynthetic pathway for producing a diterpene” refers to the biosynthetic pathway leading to a diterpene such as pleuromutilin. Said pathway at least comprises a step of converting GGPP into a diterpene or diterpene precursor and may further comprise a step of the formation of GGPP and/or one or more later steps of the synthesis of the diterpene.

As used herein, the “biosynthetic pathway for producing pleuromutilin” refers to the biosynthetic pathway leading to a pleuromutilin antibiotic such as pleuromutilin. In preferred embodiments, it refers to the biosynthetic pathway commencing with a step of the formation of geranylgeranyl pyrophosphate (GGPP), which is preferably formed from farnesyl pyrophosphate (FPP), and leading to pleuromutilin. In other preferred embodiments, it refers to the biosynthetic pathway leading from geranylgeranyl pyrophosphate (GGPP) to pleuromutilin. Said pathway has been described to include a reaction from GGPP into a pleuromutilin precursor, particularly a compound according to formula (I), as well as further reaction steps, such as those depicted in FIG. 2 (cf. Yao, 2007), which may also be referred to herein as later stages of the biosynthetic pathway. Common precursors of GGPP are isopentenyl diphosphate (IPP) and dimethylallyl diphosphate (DMAPP). As is known to the skilled person geranyl pyrophosphate (GPP) may be synthesized from IPP and DMAPP, and FPP may be synthesized from IPP and GPP.

In fungi and animals, isopentenyl diphosphate (IPP) and dimethylallyl diphosphate (DMAPP), have been described to be synthesized via the so-called the mevalonate (MVA) pathway (cf. Dewick, 2002). In the latter pathway, two molecules of acetyl-coenzyme A (acetyl-CoA) are reacted to give acetoacetyl-CoA. Acetoacetyl-CoA is reacted with a further molecule of acetyl-CoA to form 3-hydroxy-3-methylglutary-CoA (HMG-CoA). This reaction is catalyzed by HMG-CoA synthase. HMG-CoA reductase then converts HMG-CoA to mevalonic acid (MVA). The six-carbon atoms containing mevalonic acid is then transformed into the five-carbon atoms containing isopentenyl-5-pyrophosphate (IPP) by two phosphorylation reactions to yield mevalonate-5-phosphate and mevalonate-5-pyrophosphate, respectively, followed by a decarboxylation reaction to yield IPP. Isopentenyl diphosphate is isomerized by IPP isomerase to generate dimethylallyl diphosphate (DMAPP).

An alternative metabolic pathway leading to the formation of isopentenyl pyrophosphate (IPP) and dimethylallyl pyrophosphate (DMAPP) is the so-called non-mevalonate pathway or methyl erythritol phosphate pathway (MEP pathway), which starts from pyruvate and glycerinaldehyde-3-phosphate, and which is e.g. used by most bacteria and by green algae (cf. Kuzuyama, 2002). Higher plants and red algae may employ either the MVA or the MEP pathway.

In fungi, the full biosynthetic pathway for producing pleuromutilin has been described to include the reaction from FPP to geranylgeranyl pyrophosphate, which reaction is catalyzed by a particular prenyltransferase, a so-called GGPP synthase (ggs), which extends FPP with one IPP molecule. Subsequently, GGPP is reacted into a cyclic pleuromutilin precursor by a particular diterpene synthase which is also called pleuromutilin synthase. The thus obtained pleuromutilin precursor is then converted into pleuromutilin in the so-called later stages of pleuromutilin synthesis. Later stages of pleuromutilin synthesis are believed to include the actions of an acyltransferase and cytochrome P-450 enzymes.

As used herein, the expression of a polypeptide being “involved in the biosynthetic pathway for producing pleuromutilin” particularly refers to a polypeptide which is capable of catalyzing at least one of the reactions of the biosynthetic pathway for producing a pleuromutilin antibiotic, particularly pleuromutilin, and especially the pathway as defined hereinabove. Preferably, said polypeptide catalyzes at least one reaction in the conversion of FPP to a pleuromutilin antibiotic such as pleuromutilin. Preferably, said polypeptide catalyzes at least one reaction in the conversion of GGPP to a pleuromutilin antibiotic such as pleuromutilin. In preferred embodiments, the expression particularly refers to a polypeptide which is capable of catalyzing at least one of the reactions depicted in FIG. 2. Most preferably, said polypeptide is essential for at least one reaction in the overall conversion of GGPP to pleuromutilin.

A preferred polypeptide involved in the biosynthetic pathway for producing pleuromutilin is a diterpene synthase, such as those of the present invention. A particularly preferred polypeptide involved in the biosynthetic pathway for producing pleuromutilin is a pleuromutilin synthase, which is capable of catalyzing the conversion from GGPP to the tricyclic intermediate of formula (I). Most preferably, said pleuromutilin synthase is essential for the conversion from GGPP to the tricyclic intermediate of formula (I).

As used herein, the expression of a nucleic acid, gene or gene cluster being “involved in the biosynthetic pathway for producing pleuromutilin” particularly refers to a nucleic acid, gene or gene cluster encoding one or more polypeptides that is/are involved in the biosynthetic pathway for producing a pleuromutilin antibiotic such as pleuromutilin. The latter nucleic acid, gene or gene cluster are also said to be directly involved in the biosynthetic pathway for producing pleuromutilin. Nucleic acids, genes or gene clusters involved in the biosynthetic pathway for producing pleuromutilin also include ones, such as promoters or enhancers, that are indirectly involved in the biosynthetic pathway for producing pleuromutilin by acting on directly involved nucleic acids, genes or gene clusters. Preferably, a nucleic acid, gene or gene cluster involved in the biosynthetic pathway for producing pleuromutilin encodes a preferred polypeptide involved in the biosynthetic pathway for producing pleuromutilin as described herein.

As used herein “stringent hybridization conditions” and “stringent conditions” refers to conditions under which a nucleic acid molecule will hybridize to its target and to a minimal number of other sequences only.

Stringent conditions within the meaning of this invention include pre-washing in a solution of 6×SSC, 0.2% SDS at 22° C.; hybridizing at 65° C., in 6×SSC, 0.2% SDS overnight; followed by four washes at 65° C. of 30 minutes each, two in 1×SSC, 0.1% SDS and two in 0.2×SSC, 0.1% SDS.

A “method for the fermentative production” of a polypeptide is well-known to a skilled person. As used herein, it is a method involving a living system, e.g. an organism such as bacterial or a fungal organism, or a cell (culture), or a tissue (culture). The living system may be any of the hosts disclosed herein.

A “method for the fermentative production” may be characterized by only including production steps involving a living system. A “method for the fermentative production” may also be characterized by both including production steps involving a living system and production steps not involving a living system. The latter method may also be referred to herein as a “method for semisynthetic production”.

As used herein, a “method for the synthetic production” does not involve a living system, e.g. an organism such as bacterial or a fungal organism, or a cell (culture), or a tissue (culture). Preferably, such method employs chemical means or an isolated polypeptide, such as the polypeptides disclosed herein.

A “method of the production of a polypeptide” as used herein is not particularly limited. It comprises a method for the fermentative production of a polypeptide, a method for the synthetic production of a polypeptide, and any combination of the latter methods.

### SHORT DESCRIPTION OF THE FIGURES

FIG. 1 illustrates the proposed details of the reaction catalyzed by pleuromutilin synthase leading from GGDP to a compound according to formula (I) (cf. Yao, 2007).

FIG. 2 illustrates the stages of the biosynthetic pathway for producing pleuromutilin which lead from GGDP to pleuromutilin, i.e. the reaction catalyzed by the diterpene synthase as well as later stages of pleuromutilin synthesis (modified from Tsukagoshi, et al., 2007).

FIG. 3 shows the polypeptide (SEQ ID NO: 9) as well as the coding nucleic acid sequence (SEQ ID NO: 8) of the diterpene synthase (gene) involved in the biosynthetic pathway for producing pleuromutilin. Also shown is the corresponding genomic nucleic acid sequence (SEQ ID NO: 13), wherein introns are depicted in lower case as well as a sequence comprising the putative gene cluster from Clitopilus passeckerianus (SEQ ID NO: 15).

FIG. 4 depicts a proposed outline of a part of the gene cluster identified by the present inventors.

FIG. 5 depicts an amino acid alignment of the preferred diterpene synthase sequence of the invention (designated as “C.p”) with known and putative diterpene synthase sequences (Phomopsis amygdali copalyl diphosphate synthase (BAG30962), Phomopsis amygdali phyllocladan-16a-ol synthase (BAG30961), Phoma betae aphidicolan-16b-ol synthase (BAB62102), Phaeosphaeria sp. ent-kaurene synthase (BAA22426), Fusarium proliferatum ent-kaurene synthase (ABC46413), Gibberella fujikuroi ent-kaurene synthase (BAA84917), Microsporum canis ent-kaurene synthase (EEQ29644), Aspergillus niger hypothetical protein An18g02710 (XP_{—}001398730), Neosartorya fischeri hypothetical protein NFIA_{—}009790 (XP_{—}001264196))

FIG. 6 shows the deduced amino acid sequence as well as the coding nucleic acid sequence of the CYP450-1 (gene) involved in the biosynthetic pathway for producing pleuromutilin.

FIG. 7 shows the deduced amino acid sequence as well as the coding nucleic acid sequence of the acyltransferase (gene) involved in the biosynthetic pathway for producing pleuromutilin.

FIG. 8 shows the deduced amino acid sequence as well as the coding nucleic acid sequence of the geranylgeranyldiphosphate synthase (gene) involved in the biosynthetic pathway for producing pleuromutilin.

FIG. 9 shows the deduced amino acid sequence as well as the coding nucleic acid sequence of the CYP450-2 (gene) involved in the biosynthetic pathway for producing pleuromutilin.

FIG. 10 shows the deduced amino acid sequence as well as the coding nucleic acid sequence of the CYP450-3 (gene) involved in the biosynthetic pathway for producing pleuromutilin.

FIG. 11. One can assume that an increase of pleuromutilin productivity correlates with an enhanced transcription of the genes within the pleuromutilin biosynthesis cluster. Therefore the expression profiles of two strains, Clitopilus passeckerianus DSM1602 (ATCC34646, NRLL3100) and a derivative (Cp24, selected for increased pleuromutilin productivity) were analyzed (c.f. FIG. 11A). All samples were measured in triplicates. Relative transcript level values for pleuromutilin biosynthesis genes (B: CYP450-1; C: acyltransferase; D: diterpene synthase; E: GGDPS; F: CYP450-2; G: CYP450-3) were obtained after normalization of values calculated for the target genes (detector) against those of the beta actin gene as endogenous control. GAPDH (FIG. 11 H) was used as a negative control.

FIG. 12 shows the expression level of diterpene synthase measured by quantitative PCR with primer pair Cp_dts_U1 and Cp_dts_L1.

FIG. 13 shows the expression level of diterpene synthase measured by quantitative PCR with primer pair Cp_dts_U2 and Cp_dts_L2.

FIG. 14 shows the Pleuromutilin productivity of RNA interference transformants (T1-T9) containing plasmid P2543_compared to transformants containing plasmid P2558 (C1-C6) and to parental strain DSM1602

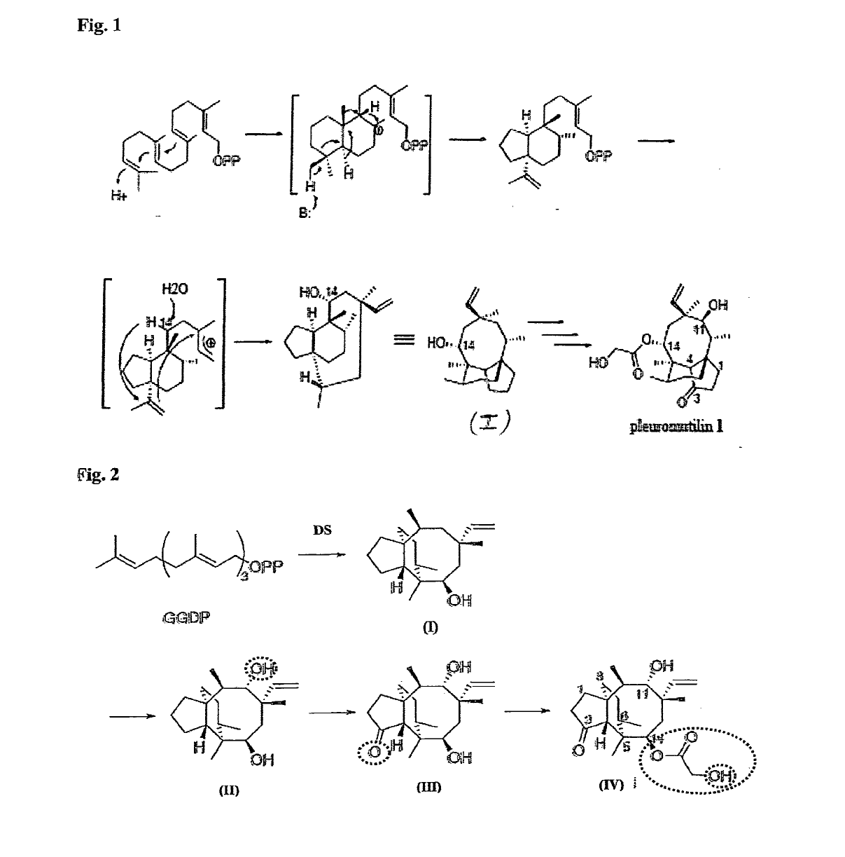

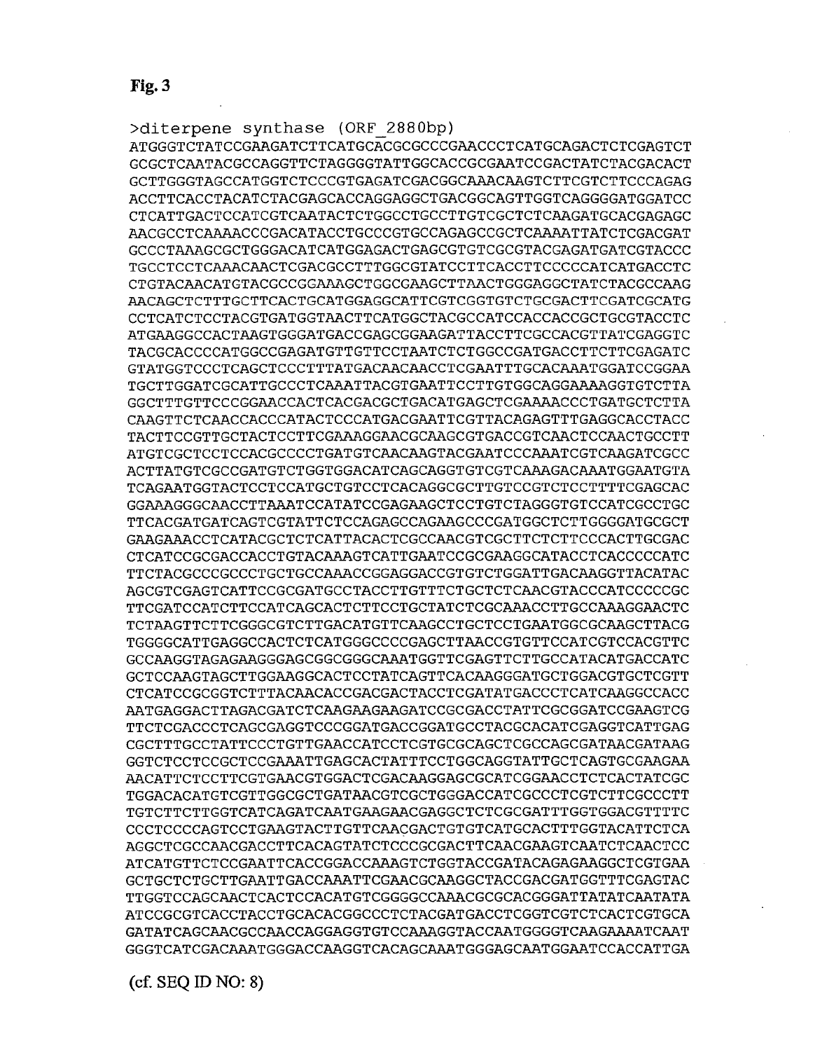

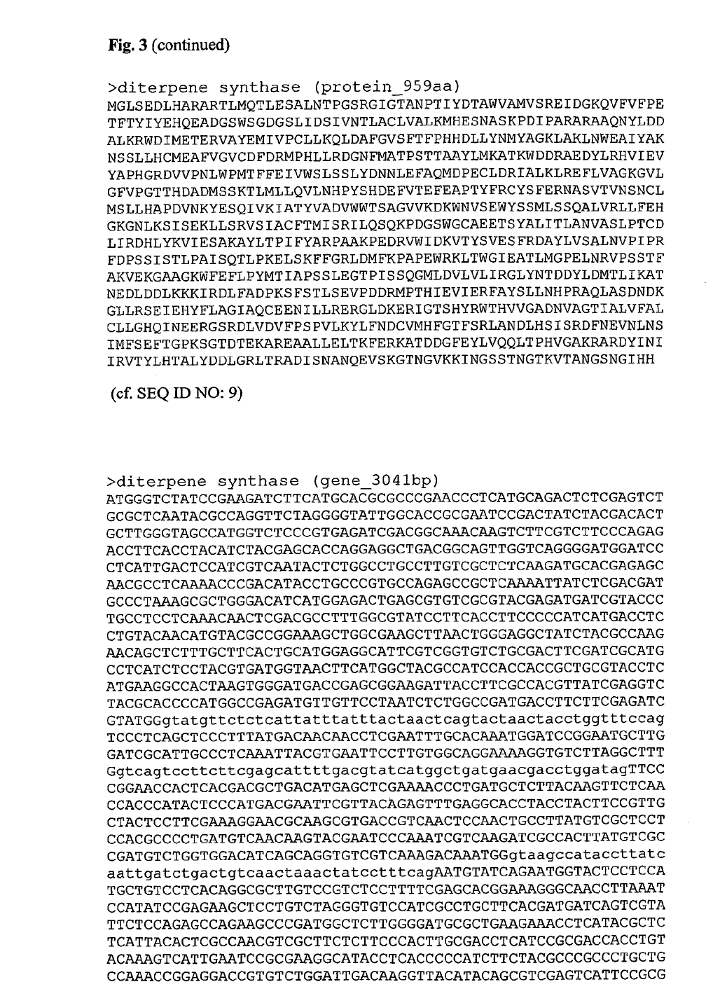

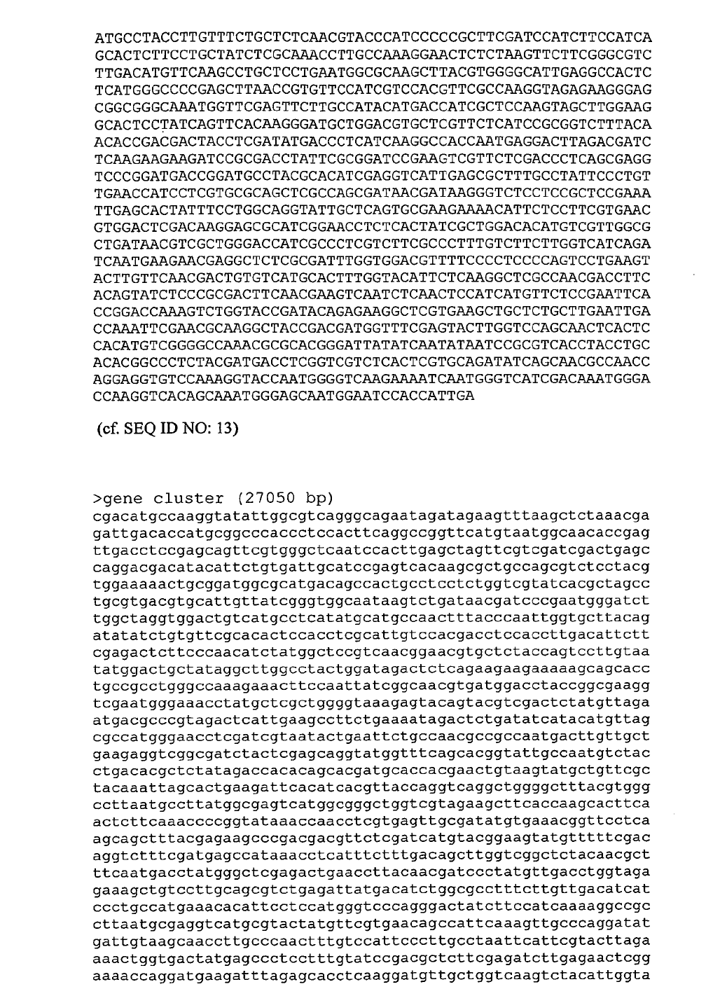

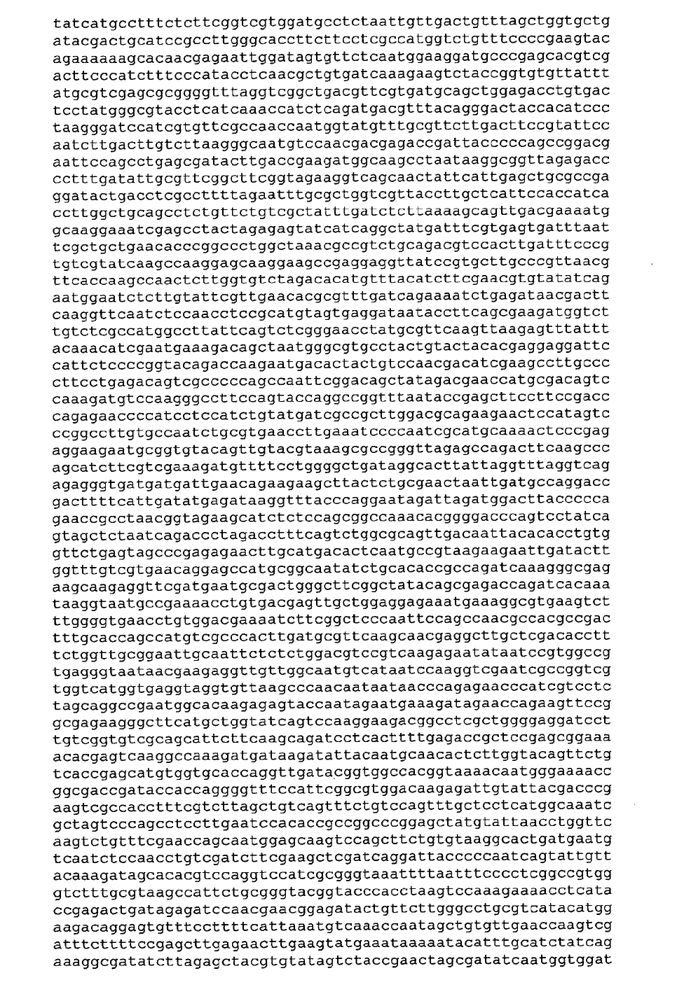

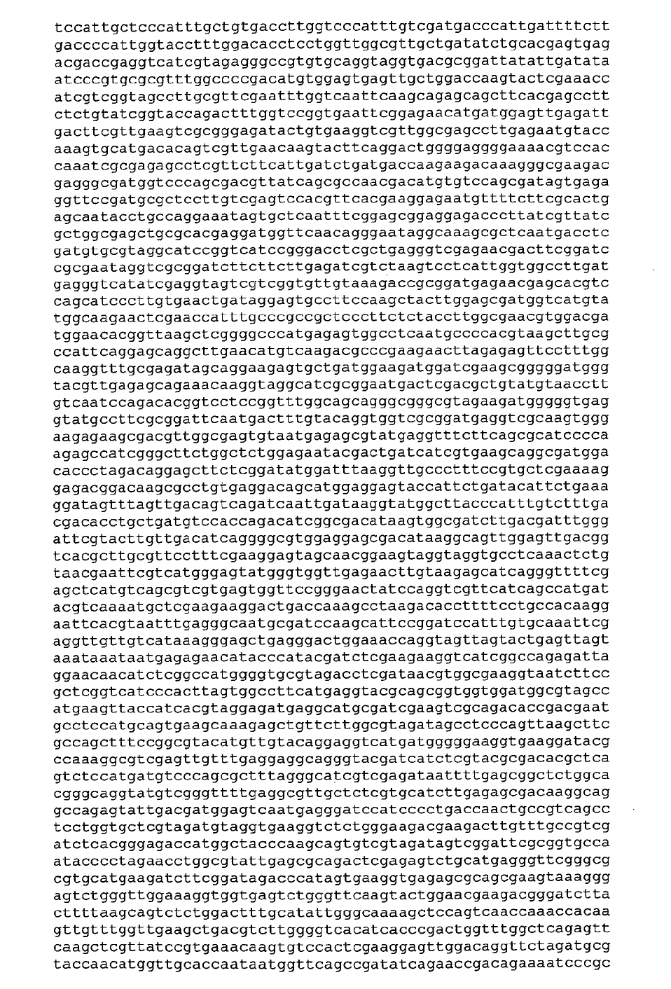

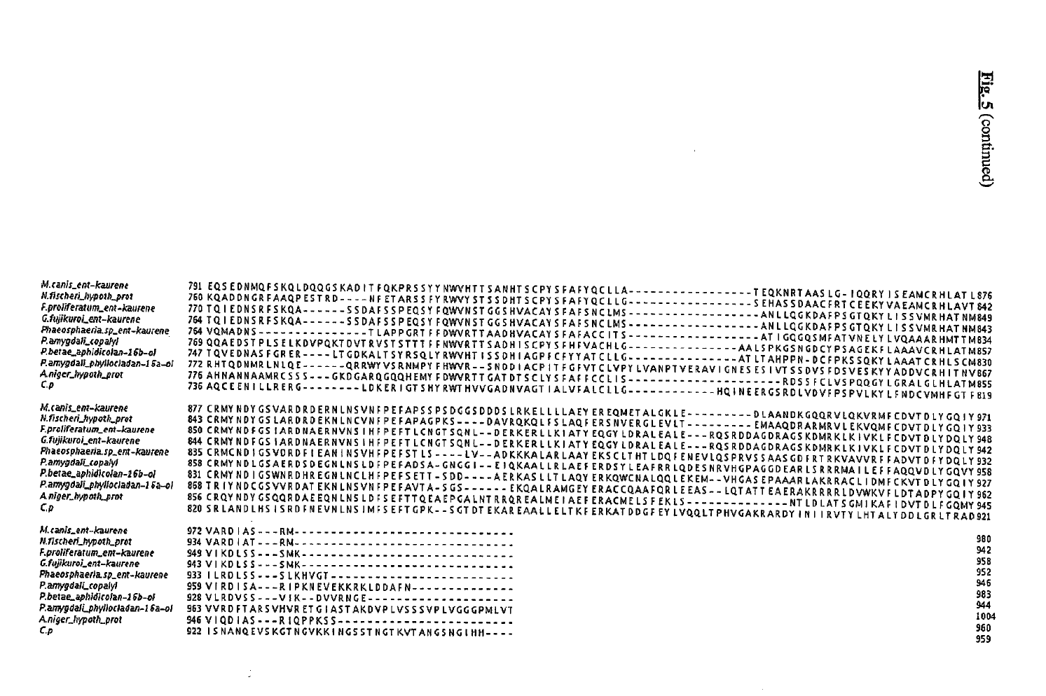

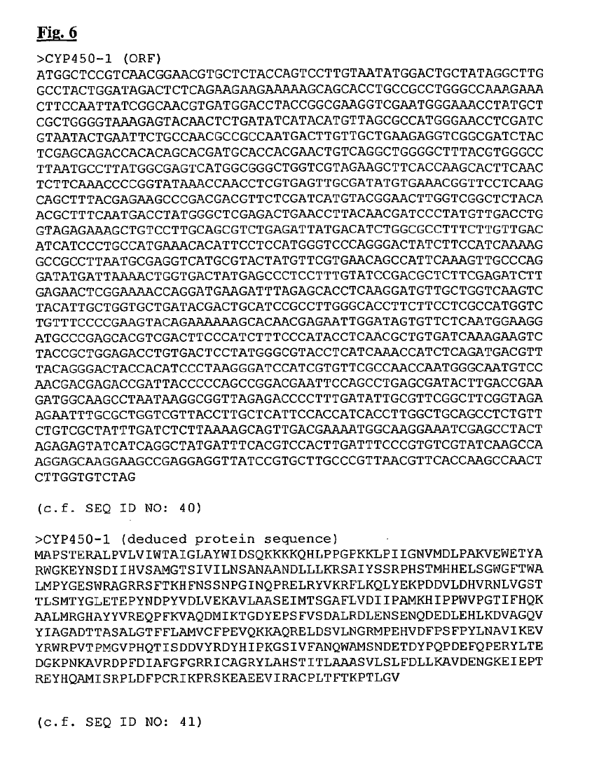

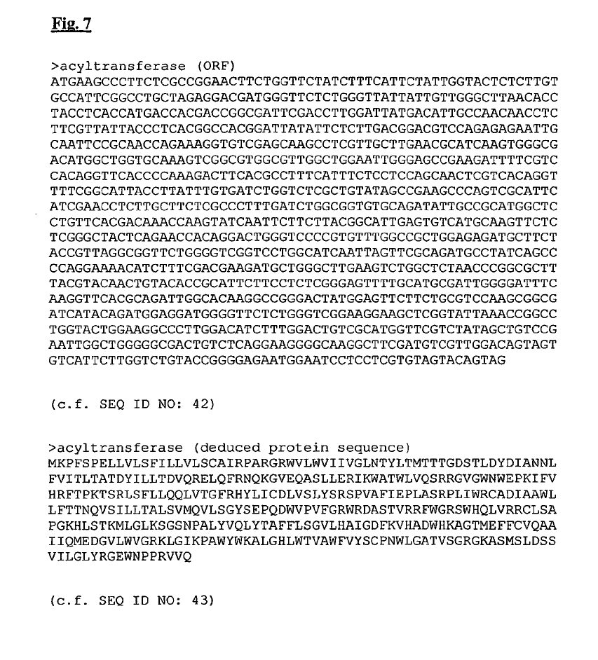

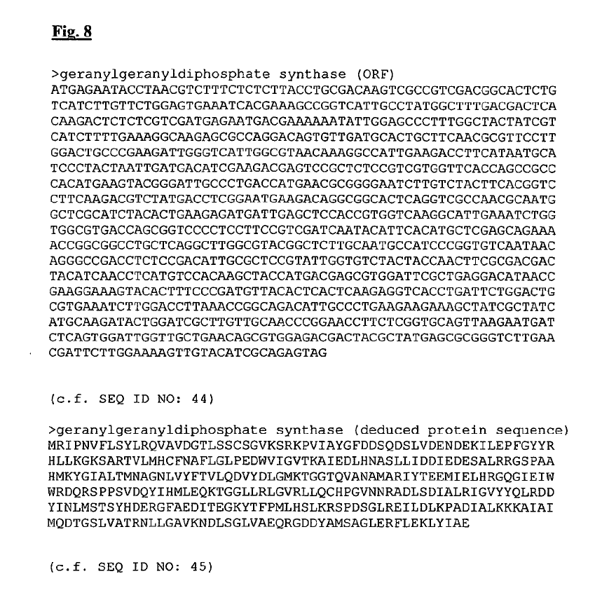

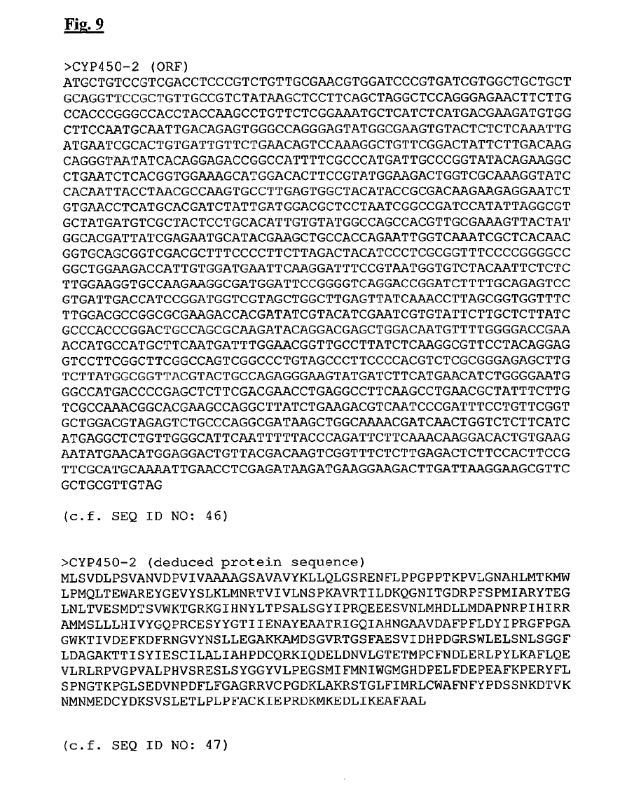

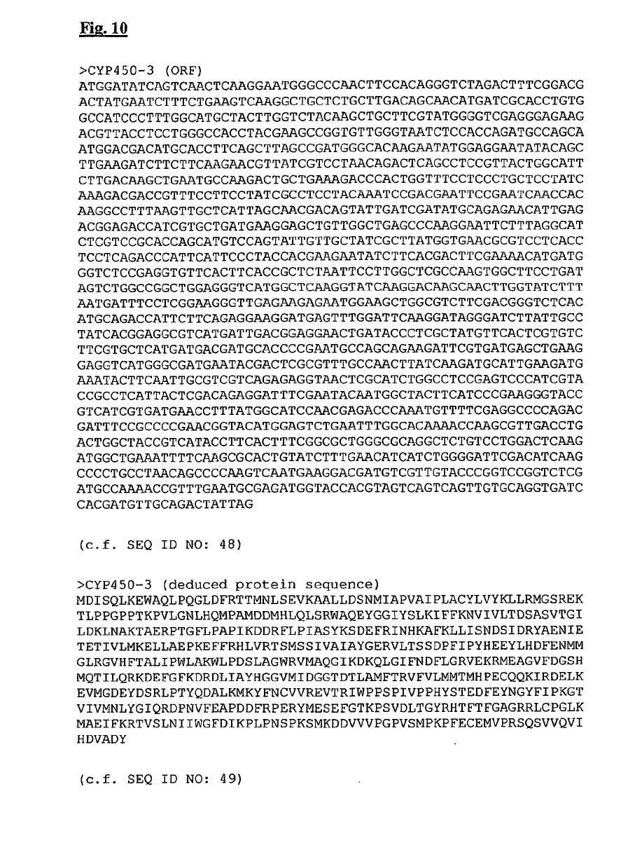

DESCRIPTION OF THE SEQUENCES

| | |
|---|---|
| SEQ ID NO: 1: | GNFMATPSTTAAYLMKATKWDDRAEDYLRHV |
| SEQ ID NO: 2: | FEAPTYFRCYSFERNASVTVNSNCLMSLL |
| SEQ ID NO: 3: | RLANDLHSISRDFNEVNLNSIMFSEF |
| SEQ ID NO: 4: | DYINIIRVTYLHTALYDDLGRLTRADISNA |
| SEQ ID NO: 5: | YSLLNHPRAQLASDNDKGLLRSEIEHYFLAG |
| SEQ ID NO: 6: | SHYRWTHVVGADNVAGTIALVFALCLLG |
| SEQ ID NO: 7: | PSSTFAKVEKGAAGKWFEFLPYMTIAPSSLEGTPI |
| SEQ ID NO: 8: | is shown in FIG. 3 |
| SEQ ID NO: 9: | is shown in FIG. 3 |
| SEQ ID NO: 10: | ggtaacttcatggctacgccatccaccaccgctgc |
| | gtacctcatgaaggccactaagtgggatgaccgag |
| | cggaagattaccttcgccacgtt |
| SEQ ID NO: 11: | tttgaggcacctacctacttccgttgctactcctt |
| | cgaaaggaacgcaagcgtgaccgtcaactccaact |
| | gccttatgtcgctcctc |
| SEQ ID NO: 12: | aggctcgccaacgaccttcacagtatctcccgcga |
| | cttcaacgaagtcaatctcaactccatcatgttct |
| | ccgaattc |
| SEQ ID NO: 13 | are shown in FIG. 3 |
| and 15: | |
| SEQ ID NO: 16: | caatgaccta tgggctcgag actgaa |
| SEQ ID NO: 17: | gttgaggtat gggaaagatg ggaagtc |
| SEQ ID NO: 18: | tctgagatta tgacatctgg cgccttt |
| SEQ ID NO: 19: | gtgcccaagg cggatgcagt cgt |
| SEQ ID NO: 20: | ctggaattgg gagccgaaga ttt |
| SEQ ID NO: 21: | gagaacccca tcctccatct gtatgat |
| SEQ ID NO: 22: | cgtcacaggt tttcggcatt acctta |
| SEQ ID NO: 23: | cgagaggaag aatgcggtgt acagt |
| SEQ ID NO: 24: | cccatgacga attcgttaca gagttt |
| SEQ ID NO: 25: | cttcgcggat tcaatgactt tgtaca |
| SEQ ID NO: 26: | ctgatgtcaa caagtacgaa tcccaaa |
| SEQ ID NO: 27: | tcgggcttct ggctctggag aat |
| SEQ ID NO: 28: | agtccgctct ccgtcgtggt tca |
| SEQ ID NO: 29: | agcttgtgga catgaggttg atgtagt |
| SEQ ID NO: 30: | caagacgtct atgacctcgg aatgaa |
| SEQ ID NO: 31: | gagccgtacg ccaagcctga gca |
| SEQ ID NO: 32: | ttcttagact acatccctcg cggttt |
| SEQ ID NO: 33: | caaccgttcc aaatcattga agcat |
| SEQ ID NO: 34: | attccggggt caggaccgga tct |
| SEQ ID NO: 35: | cgattcgatg tacgatatcg tggtctt |
| SEQ ID NO: 36: | gcgtcatgat tgacggagga act |
| SEQ ID NO: 37: | cagccatctt gagtccagga caga |
| SEQ ID NO: 38: | ggcgatgaat acgactcgcg ttt |
| SEQ ID NO: 39: | catgtaccgt tcggggcgga aat |
| SEQ ID NO: 40: | is shown in FIG. 6. |
| SEQ ID NO: 41: | is shown in FIG. 6. |
| SEQ ID NO: 42: | is shown in FIG. 7. |
| SEQ ID NO: 43: | is shown in FIG. 7. |
| SEQ ID NO: 44: | is shown in FIG. 8. |
| SEQ ID NO: 45: | is shown in FIG. 8. |
| SEQ ID NO: 46: | is shown in FIG. 9. |
| SEQ ID NO: 47: | is shown in FIG. 9. |
| SEQ ID NO: 48: | is shown in FIG. 10. |
| SEQ ID NO: 49: | is shown in FIG. 10. |
| SEQ ID NO: 50: | tgatggtcaa gttatcacga ttgg |
| SEQ ID NO: 51: | gagttgtaag tggtttcgtg aatacc |
| SEQ ID NO: 52: | tcggctctac aacgctttca |
| SEQ ID NO: 53: | tgtcataatc tcagacgctg caa |
| SEQ ID NO: 54: | aagattttcg tccacaggtt cac |
| SEQ ID NO: 55: | tacagcgaga ccagatcaca aataa |
| SEQ ID NO: 56: | gttacagagt ttgaggcacc tacct |
| SEQ ID NO: 57: | cgtggaggag cgacataagg |
| SEQ ID NO: 58: | gacatcgaag acgagtccgc |
| SEQ ID NO: 59: | ttgaaggacc gtgaagtaga caag |
| SEQ ID NO: 60: | tacatccctc gcggtttcc |
| SEQ ID NO: 61: | ggtcttccag ccg |
| SEQ ID NO: 62: | gtcatgattg acggaggaac tg |
| SEQ ID NO: 63: | tccttcagct catcacgaat ctt |
| SEQ ID NO: 64: | is shown in Example 4. |
| SEQ ID NO: 65: | is shown in Example 4. |
| SEQ ID NO: 66: | is shown in Example 4. |
| SEQ ID NO: 67: | is shown in Example 4. |
| SEQ ID NO: 68: | is shown in Example 4. |
| SEQ ID NO: 69: | is shown in Example 4. |
| SEQ ID NO: 70: | AATCGTCAAGATCGCCACTTATG |
| SEQ ID NO: 71: | GAGTACCATTCTGATACATTCCATTTG |

SEQ ID NOs: 1 to 3 are amino acid sequences of conserved regions of a preferred polypeptide, more precisely a preferred diterpene synthase, of the invention, whereas SEQ ID NOs: 4 to 7 relate to signature regions of said polypeptide.

SEQ ID NO: 9 (cf. also SEQ ID NO: 14) shows the supposed polypeptide sequence of the diterpene synthase involved in the production of pleuromutilin of Clitopilus passseckerianus.

SEQ ID NO: 8 shows the corresponding nucleotide sequence of the diterpene synthase cDNA involved in the production of pleuromutilin of Clitopilus passseckerianus. SEQ ID NO: 8 is also comprised in the reverse complementary strand (i.e. the (−) strand) of SEQ ID NO: 15.

SEQ ID NOs: 10 to 12 are nucleic acid sequences encoding the conserved regions of SEQ ID NOs: 1 to 3.

SEQ ID NO: 13 shows the supposed gene sequence of the diterpene synthase involved in the production of pleuromutilin of Clitopilus passseckerianus.

SEQ ID NO: 15 shows a nucleic acid sequence which is contemplated to comprise a gene cluster involved in the biosynthetic pathway for producing a diterpenoid, more precisely pleuromutilin. Said nucleic acid sequence is derived from the genome of Clitopilus passseckerianus.

SEQ ID Nos: 16-39 are the primers and nested primers used in RACE, as set forth in Example 3 below.

SEQ ID NO: 40 shows the supposed open reading frame of CYP450-1 involved in the production of pleuromutilin of Clitopilus passseckerianus. SEQ ID NO: 41 is the deduced amino acid sequence of CYP 450-1.

SEQ ID NO: 42 shows the supposed open reading frame of acyltransferase involved in the production of pleuromutilin of Clitopilus passseckerianus. SEQ ID NO: 43 is the deduced amino acid sequence of acyltransferase.

SEQ ID NO: 44 shows the supposed open reading frame of GGDPS involved in the production of pleuromutilin of Clitopilus passseckerianus. SEQ ID NO: 45 is the deduced amino acid sequence of GGDPS.

SEQ ID NO: 46 shows the supposed open reading frame of CYP450-2 involved in the production of pleuromutilin of Clitopilus passseckerianus. SEQ ID NO: 47 is the deduced amino acid sequence of CYP 450-2.

SEQ ID NO: 48 shows the supposed open reading frame of CYP450-3 involved in the production of pleuromutilin of Clitopilus passseckerianus. SEQ ID NO: 49 is the deduced amino acid sequence of CYP 450-3.

SEQ ID NOs: 50-63 are the primers used in quantitative PCR expression analysis, as set forth in Example 3 below.

SEQ ID NOs: 64-66 define the RNAi cassette of P2543_Hairpin.

SEQ ID NOs: 67 and 68 show the promotor and terminator used for efficient transcription of the hairpin cassette.

SEQ ID NO: 69 was used to construct P2558, as set forth in Example 4 below.

SEQ ID NOs: 70 and 71 are the primers used in quantitative PCR expression analysis, as set forth in Example 4 below.

### DETAILED DESCRIPTION OF PREFERRED ASPECTS AND EMBODIMENTS

The present invention particularly relates to compounds such as polypeptides and nucleic acid molecules, as well as methods and uses as defined in the claims.

Generally, in a first aspect, the present invention relates to novel isolated polypeptides, particularly to novel diterpene synthases, in particular pleuromutilin synthases.

In one embodiment, the polypeptide comprises an amino acid sequence which amino acid sequence comprises a sequence having at least 50% sequence identity to SEQ ID NO: 1, a sequence having at least 40% sequence identity to SEQ ID NO: 2, and at least one sequence selected from the group consisting of i) a sequence having at least 15% sequence identity to SEQ ID NO: 7; ii) a sequence having at least 25% sequence identity to SEQ ID NO: 4; iii) a sequence having at least 45% sequence identity to SEQ ID NO: 5; and iv) a sequence having at least 45% sequence identity to SEQ ID NO: 6, wherein SEQ ID NOs: 1-2 and 4-7 are of Clitopilus passeckerianus origin. The amino acid sequence of said isolated polypeptide may further comprise a sequence having at least 50% sequence identity to SEQ ID NO: 3. SEQ ID NOs: 1 to 7 are of Clitopilus passseckerianus origin.

In another embodiment, the polypeptide comprises an amino acid sequence which amino acid sequence comprises a sequence having at least 50% sequence identity to SEQ ID NO: 3, a sequence having at least 40% sequence identity to SEQ ID NO: 2, and at least one sequence selected from the group consisting of i) to iv) as defined above.

In another embodiment, the polypeptide comprises an amino acid sequence which amino acid sequence comprises a sequence having at least 50% sequence identity to SEQ ID NO: 1, a sequence having at least 50% sequence identity to SEQ ID NO: 3, and at least one sequence selected from the group consisting of i) to iv) as defined above.

In another embodiment, the polypeptide comprises an amino acid sequence which amino acid sequence comprises a sequence having at least 60%, particularly at least 70% sequence identity to SEQ ID NO: 9, more preferably at least 80%, even more preferably at least 85%, or even at least 90%, such as even more preferably at least 95% sequence identity to SEQ ID NO: 9.

The molecular weight of the isolated polypeptide define herein is preferably between 90 kDa and 140 kDa, particularly between 100 kDa and 130 kDa, especially between 105 kDa and 120 kDa.

In particular embodiments, the polypeptide comprises an amino acid sequence which amino acid sequence comprises SEQ ID NO: 1 and SEQ ID NO: 2, and at least one sequence selected from the group consisting of i) to iv) as defined above, especially at least one sequence selected from the group consisting of i′) SEQ ID NO: 7; ii′) SEQ ID NO: 4; iii′) SEQ ID NO: 5; and iv′) SEQ ID NO: 6. The amino acid sequence may further comprise SEQ ID NO: 3.

The isolated polypeptide of the invention preferably has diterpene synthase activity, especially pleuromutilin synthase activity. Accordingly, the isolated polypeptide is preferably involved in the biosynthetic pathway for producing pleuromutilin and is preferably capable of catalyzing the conversion of geranylgeranyl pyrophosphate into a pleuromutilin precursor, particularly into a compound according to formula (I). More preferably it is essential in pleuromutilin producing organisms for the production of pleuromutilin.

As mentioned above, the present inventors have succeeded in identifying a nucleic acid sequence which is contemplated to comprise a gene cluster derived from the genome of Clitopilus passeckerianus involved in the biosynthetic pathway for producing a diterpenoid, more precisely pleuromutilin. Said nucleic acid sequence is envisaged to comprise at least six transcriptionally co-regulated open reading frames encoding polypeptides which are thought to be involved in pleuromutilin biosynthesis, namely an acyltransferase (AT), a geranylgeranyldiphosphate synthase (GGDPS) and three cytochrome P450 enzymes. Typically, hydroxyl or other oxygen functionalities are introduced via the action of a monooxygenase such as cytochrome P450 monooxygenases. The resulting hydroxyl group might be further modified by acylation, alkylation and glycosylation. In the subsequent reactions of the pathway for producing pleuromutilin, the actions of cytochrome P-450 enzymes (acts, for example, on C3 and C11) and an acyltransferase (acts, for example, on C14 hydroxyl) are considered necessary to complete formation of pleuromutilin (cf. Yao, 2007).

Thus, in another embodiment, the polypeptide comprises an amino acid sequence which amino acid sequence comprises a sequence having at least 60%, particularly at least 70% sequence identity to SEQ ID NO: 43, more preferably at least 80%, even more preferably at least 85%, or even at least 90%, such as even more preferably at least 95% sequence identity to SEQ ID NO: 43. This isolated polypeptide preferably has acyltransferase activity. Accordingly, the isolated polypeptide is preferably involved in the biosynthetic pathway for producing pleuromutilin and preferably acts, for example, on the C14 hydroxyl, e.g. in the conversion from a compound of formula (III) into a compound of formula (IV), as shown in FIG. 2. More preferably it is essential in pleuromutilin producing organisms for the production of pleuromutilin. In preferred embodiments, an acyltransferase of the invention has at least 5%, 10% or 20%, preferably at least 30% or 40%, such as at least 50%, or at least 60%, 70%, 80%, particularly at least 90% or 95%, especially at least 100%, such as at least 125%, 150% or 175% of the activity of the acyltransferase of SEQ ID NO: 43, particularly at least 2-fold or 5-fold, at least 10-fold or 25-fold, such as from 50- to 100-fold the activity of the acyltransferase of SEQ ID NO: 43. Any suitable method may be used in order to determine the activity of the variant acyltransferase in comparison to the acyltransferase of SEQ ID NO: 45, including measuring the increase in concentration of a compound of formula (IV) over the time, or the decrease of a suitable substrate, e.g. acyl-CoA or a compound of formula (III), when incubated under identical conditions, which allow acyltransferase activity. Exemplary assays may involve product formation by employing [³H]-labeled substrates, and/or detection by GC-MS. However, suitable assays which may be used are generally known in the art.

In another embodiment, the polypeptide comprises an amino acid sequence which amino acid sequence comprises a sequence having at least 60%, particularly at least 70% sequence identity to SEQ ID NO: 45, more preferably at least 80%, even more preferably at least 85%, or even at least 90%, such as even more preferably at least 95% sequence identity to SEQ ID NO: 45. This isolated polypeptide preferably has geranylgeranyldiphosphate synthase activity. Accordingly, the isolated polypeptide is preferably involved in the biosynthetic pathway for producing pleuromutilin and is preferably involved in the formation of geranylgeranyl pyrophosphate (GGPP), which is preferably formed from farnesyl pyrophosphate (FPP). More preferably it is essential in pleuromutilin producing organisms for the production of pleuromutilin. In preferred embodiments, an geranylgeranyldiphosphate synthase of the invention has at least 5%, 10% or 20%, preferably at least 30% or 40%, such as at least 50%, or at least 60%, 70%, 80%, particularly at least 90% or 95%, especially at least 100%, such as at least 125%, 150% or 175% of the activity of the geranylgeranyldiphosphate synthase of SEQ ID NO: 45, particularly at least 2-fold or 5-fold, at least 10-fold or 25-fold, such as from 50- to 100-fold the activity of the geranylgeranyldiphosphate synthase of SEQ ID NO: 45.

Any suitable method may be used in order to determine the activity of the variant geranylgeranyldiphosphate synthase in comparison to the geranylgeranyldiphosphate synthase of SEQ ID NO: 45, including measuring the increase in concentration of GGPP over the time, or the decrease of a suitable substrate, e.g. FPP, when incubated with a suitable substrate, e.g. FPP, under identical conditions, which allow GGPP formation. Exemplary assays may involve product formation by employing [³H]-labeled substrates, and/or detection by GC-MS. Further assays, which may be suitable for determining GGDPS activity are e.g. described in Chang et al. (2006) Crystal structure of type-III geranylgeranyl pyrophosphate synthase from Saccharomyces cerevisiae and the mechanism of product chain length determination. Journal of Biological Chemistry; 281(21):14991-15000; and Singkaravanit S. et al. (2010) Geranylgeranyl diphosphate synthase genes in entomopathogenic fungi. Appl Microbiol Biotechnol; 85(5):1463-1472. However, suitable assays which may generally be used are known in the art.

In still another embodiment, the polypeptide comprises an amino acid sequence which amino acid sequence comprises a sequence having at least 60%, particularly at least 70% sequence identity to SEQ ID NO: 41, more preferably at least 80%, even more preferably at least 85%, or even at least 90%, such as even more preferably at least 95% sequence identity to SEQ ID NO: 41.

Alternatively, the polypeptide comprises an amino acid sequence which amino acid sequence comprises a sequence having at least 60%, particularly at least 70% sequence identity to SEQ ID NO: 47, more preferably at least 80%, even more preferably at least 85%, or even at least 90%, such as even more preferably at least 95% sequence identity to SEQ ID NO: 47; or

the polypeptide comprises an amino acid sequence which amino acid sequence comprises a sequence having at least 60%, particularly at least 70% sequence identity to SEQ ID NO: 49, more preferably at least 80%, even more preferably at least 85%, or even at least 90%, such as even more preferably at least 95% sequence identity to SEQ ID NO: 49. This isolated polypeptide preferably has cytochrome P450 activity, e.g. monooxygenase activity. Accordingly, the isolated polypeptide is preferably involved in the biosynthetic pathway for producing pleuromutilin and preferably acts, for example, on the C3 and C11 of the pleuromutilin precursor, such as a pleuromutilin precursor shown in formula (I) or (II) in FIG. 2. Thus, the cytochrome P450 is preferably a monooxygenase. Preferably, said cytochrome P450 is involved in the conversion of a compound of formula (I) into a compound of formula (II) and/or in the conversion of a compound of formula (II) into a compound of formula (III), as shown in FIG. 2. More preferably it is essential in pleuromutilin producing organisms for the production of pleuromutilin. In preferred embodiments, said cytochrome P450 enzyme has at least 5%, 10% or 20%, preferably at least 30% or 40%, such as at least 50%, or at least 60%, 70%, 80%, particularly at least 90% or 95%, especially at least 100%, such as at least 125%, 150% or 175% of the activity of the corresponding cytochrome P450 encoded by SEQ ID NO: 41, 47, and 49, respectively, particularly at least 2-fold or 5-fold, at least 10-fold or 25-fold, such as from 50- to 100-fold the activity of the corresponding cytochrome P450 encoded by SEQ ID NO: 41, 47, and 49, respectively. Any suitable method may be used in order to determine the activity of the variant cytochrome P450 enzyme in comparison to the cytochrome P450 enzyme of SEQ ID NO: 41, 47 or 49, respectively, including measuring the increase in concentration of a compound of formula (II) or (III) over the time, or the decrease of a suitable substrate, e.g. a compound of formula (I) or (II), when incubated under identical conditions, which allow cytochrome P450 activity. Exemplary assays may involve product formation by employing [³H]-labeled substrates, and/or detection by GC-MS. However, suitable assays which may be used are known in the art.

In certain preferred embodiments, the polypeptides of the invention are non-natural polypeptides.

According to a second aspect, there is provided an isolated nucleic acid molecule comprising

A) a nucleotide sequence encoding a polypeptide according to any one of claims 1 to 4 or a polypeptide of SEQ ID NO: 9,

B) a nucleotide sequence which is
a) the sequence of SEQ ID NO: 8; or
a′) the sequence of SEQ ID NO: 15 or the sequence complementary thereto; or
b) a partial sequence of a sequence defined in a′), which partial sequence encodes a diterpene synthase; or
c) a sequence which encodes a diterpene synthase and has at least 40% sequence identity to a sequence defined in a′) or has at least 60% sequence identity to the sequence defined in a) or the partial sequence defined in b); or
d) a sequence which encodes a diterpene synthase and which is degenerate as a result of the genetic code to a sequence defined in any one of a), a′), b) and c); or
e) a sequence which encodes a diterpene synthase and which is capable of hybridizing to SEQ ID NO: 8 and/or SEQ ID NO: 13 under stringent conditions,

C) at least 18 consecutive nucleotides of a nucleotide sequence as defined in item B, and/or

D) at least 18 consecutive nucleotides and capable of hybridizing to a nucleic acid molecule having a nucleotide sequence as defined in item A or item B under stringent conditions.

In one preferred embodiment, the isolated nucleic acid molecule comprises A) as defined above. In another preferred embodiment, the isolated nucleic acid molecule comprises any one of B), such as Ba, Ba′, Bb, Bc, Bd, Be, as defined above.

Generally, preferred nucleic acid molecules of the invention encode a diterpene synthase, more preferably a pleuromutilin synthase; and/or encode a polypeptide having diterpene synthase activity, more preferably a polypeptide having pleuromutilin synthase activity. Additionally or alternatively, the isolated nucleic acid molecule may (also) comprise

A) a nucleotide sequence encoding a polypeptide according to a polypeptide of SEQ ID NO: 43 or a polypeptide having acyltransferase activity and comprising an amino acid sequence having at least 60% sequence identity to SEQ ID NO: 43, as defined above,

B) a nucleotide sequence which is
a) the sequence of SEQ ID NO: 42; or
b) a partial sequence of SEQ ID NO: 15 or the sequence complementary thereto, which partial sequence encodes an acyltransferase; or
c) a sequence which encodes an acyltransferase and has at least 60% sequence identity to the sequence defined in a) or the partial sequence defined in b); or
d) a sequence which encodes an acyltransferase and which is degenerated as a result of the genetic code to a sequence defined in any one of a), b) and c); or
e) a sequence which encodes an acyltransferase and which is capable of hybridizing to SEQ ID NO: 42 under stringent conditions.

Preferably, the acyltransferase is involved in the production of pleuromutilin or a pleuromutilin precursor, such as a compound of formula (IV), as shown in FIG. 2. Additionally or alternatively, the isolated nucleic acid molecule may (also) comprise

A) a nucleotide sequence encoding a polypeptide according to a polypeptide of SEQ ID NO: 45 or a polypeptide having geranylgeranyldiphosphate synthase activity and comprising an amino acid sequence having at least 60% sequence identity to SEQ ID NO: 45, as defined above,

B) a nucleotide sequence which is
a) the sequence of SEQ ID NO: 44; or
b) a partial sequence of SEQ ID NO: 15 or the sequence complementary thereto, which partial sequence encodes a geranylgeranyldiphosphate synthase; or
c) a sequence which encodes a geranylgeranyldiphosphate synthase and has at least 60% sequence identity to the sequence defined in a) or the partial sequence defined in b); or
d) a sequence which encodes a geranylgeranyldiphosphate synthase and which is degenerated as a result of the genetic code to a sequence defined in any one of a), b) and c); or
e) a sequence which encodes a geranylgeranyldiphosphate synthase and which is capable of hybridizing to SEQ ID NO: 44 under stringent conditions.

Additionally or alternatively, the isolated nucleic acid molecule may (also) comprise

A) a nucleotide sequence encoding a polypeptide according to a polypeptide of SEQ ID NO: 41 or a polypeptide having cytochrome P450 activity and comprising an amino acid sequence having at least 60% sequence identity to SEQ ID NO: 41, as defined above,

B) a nucleotide sequence which is
a) the sequence of SEQ ID NO: 40; or
b) a partial sequence of SEQ ID NO: 15 or the sequence complementary thereto, which partial sequence encodes a cytochrome P450; or
c) a sequence which encodes a cytochrome P450 and has at least 60% sequence identity to the sequence defined in a) or the partial sequence defined in b); or
d) a sequence which encodes a cytochrome P450 and which is degenerated as a result of the genetic code to a sequence defined in any one of a), b) and c); or
e) a sequence which encodes a cytochrome P450 and which is capable of hybridizing to SEQ ID NO: 40 under stringent conditions.

Additionally or alternatively, the isolated nucleic acid molecule may (also) comprise

A) a nucleotide sequence encoding a polypeptide according to a polypeptide of SEQ ID NO: 47 or a polypeptide having cytochrome P450 activity and comprising an amino acid sequence having at least 60% sequence identity to SEQ ID NO: 47, as defined above,

B) a nucleotide sequence which is
a) the sequence of SEQ ID NO: 46; or
b) a partial sequence of SEQ ID NO: 15 or the sequence complementary thereto, which partial sequence encodes a cytochrome P450; or
c) a sequence which encodes a cytochrome P450 and has at least 60% sequence identity to the sequence defined in a) or the partial sequence defined in b); or
d) a sequence which encodes a cytochrome P450 and which is degenerated as a result of the genetic code to a sequence defined in any one of a), b) and c); or
e) a sequence which encodes a cytochrome P450 and which is capable of hybridizing to SEQ ID NO: 46 under stringent conditions.

Additionally or alternatively, the isolated nucleic acid molecule may (also) comprise

A) a nucleotide sequence encoding a polypeptide according to a polypeptide of SEQ ID NO: 49 or a polypeptide having cytochrome P450 activity and comprising an amino acid sequence having at least 60% sequence identity to SEQ ID NO: 49, as defined above,

B) a nucleotide sequence which is
a) the sequence of SEQ ID NO: 48; or
b) a partial sequence of SEQ ID NO: 15 or the sequence complementary thereto, which partial sequence encodes a cytochrome P450; or
c) a sequence which encodes a cytochrome P450 and has at least 60% sequence identity to the sequence defined in a) or the partial sequence defined in b); or
d) a sequence which encodes a cytochrome P450 and which is degenerated as a result of the genetic code to a sequence defined in any one of a), b) and c); or
e) a sequence which encodes a cytochrome P450 and which is capable of hybridizing to SEQ ID NO: 48 under stringent conditions.

Preferably, the cytochrome P450 activity is monooxygenase activity, more preferably involved in the production of a pleuromutilin precursor, such as a compound of formula (II) and/or a compound of formula (III), as shown in FIG. 2.

As used herein, a “sequence which encodes a polypeptide having diterpene synthase activity” is preferably a sequence which encodes a diterpene synthase. Preferably, a sequence which encodes a diterpene synthase is a sequence which encodes a pleuromutilin synthase.

Also, as used herein, a “sequence which encodes a polypeptide having diterpene synthase activity” is preferably a sequence which encodes a polypeptide having pleuromutilin synthase activity. Preferably, a sequence which encodes a polypeptide having pleuromutilin synthase activity is a sequence which encodes a pleuromutilin synthase. In one additional embodiment, the isolated nucleic acid molecule comprises C) as defined above. In one additional embodiment, the isolated nucleic acid molecule comprises D) as defined above. Said at least 18 consecutive nucleotides of C) or D) are preferably at least 19, 20, 25, particularly at least 30, 35, 40, 45, particularly at least 50, 55, 60, 65, particularly at least 70, 75, 80, 85, 90, 95, particularly at least 100, 150, 200, 250, particularly at least 300, 350, 400, 450, or 500 consecutive nucleotides.

The sequence according to Ba′ was obtained by isolating and sequencing genomic DNA of Clitopilus passeckerianus.

In preferred embodiments, the nucleic acid molecule of the invention comprises a nucleotide sequence which nucleotide sequence comprises a sequence having at least 70% sequence identity to SEQ ID NO: 8, more preferably at least 80%, even more preferably at least 85%, or even at least 90%, such as even more preferably at least 95% sequence identity to SEQ ID NO: 8.

In other preferred embodiments, the nucleic acid molecule of the invention comprises a nucleotide sequence which nucleotide sequence comprises a sequence having at least 50%, particularly at least 60%, especially at least 70% sequence identity to SEQ ID NO: 15, more preferably at least 80%, even more preferably at least 85%, or even at least 90%, such as even more preferably at least 95% sequence identity to SEQ ID NO: 15. In certain preferred embodiments, the nucleic acid molecule of the invention comprises a gene cluster involved in a biosynthetic pathway for producing a diterpene, particularly for producing pleuromutilin.

The isolated polypeptides and the isolated nucleic acid molecules are preferably derivable from a fungal host, particularly a fungus from the division basidomycota, more particularly from the order agaricales, even more particularly from the family entolomataceae, especially from the genus Clitopilus, particularly from the group consisting of Clitopilus scyphoides, Clitopilus prunulus, Clitopilus hobsonii, Clitopilus pseudo-pinsitus, Clitopilus pinsitus and Clitopilus passeckerianus, in particular from Clitopilus pinsitus or Clitopilus passeckerianus, or from the genus Pleurotus.

In certain preferred embodiments, the nucleic acid molecules of the invention are non-natural nucleic acid molecules.

The nucleic acid molecule of the invention may further comprise one or more regulatory sequences e.g. selected from promoters, particularly strong promoters, enhancers, repressor binding sites, internal ribosomal entry sites (IRES), and terminators, or may further comprise any combination thereof. If the nucleic acid molecule comprises more than one nucleic acid sequences according to the invention involved in a diterpene synthesis pathway, such as the pleuromutilin biosynthetic pathway, said nucleic acid sequences may be in tandem orientation, or may be polycistronic. Regulatory sequences for tandem expression or polycistronic expression are known in the art. The nucleic acid molecule of the invention may encode a diterpene synthase according to the invention involved in the pleuromutilin biosynthetic pathway. Alternatively, the nucleic acid molecule may encode an acyltransferase according to the invention involved in the pleuromutilin biosynthetic pathway. Alternatively, the nucleic acid molecule may encode a geranylgeranyl synthase according to the invention involved in the pleuromutilin biosynthetic pathway. Alternatively, the nucleic acid molecule may encode at least one cytochrome P450 enzyme, e.g. a monooxoygenase, according to the invention involved in the pleuromutilin biosynthetic pathway. Alternatively, the nucleic acid molecule may encode both a diterpene synthase according to the invention and an acyltransferase according to the invention involved in the pleuromutilin biosynthetic pathway. Alternatively, the nucleic acid molecule may encode both a diterpene synthase according to the invention and a geranylgeranyl synthase according to the invention involved in the pleuromutilin biosynthetic pathway. Alternatively, the nucleic acid molecule may encode both a diterpene synthase according to the invention and at least one cytochrome P450 enzyme according to the invention involved in the pleuromutilin biosynthetic pathway. However, also contemplated is a nucleic acid molecule, which may encode both an acyltransferase according to the invention and at least one cytochrome P450 enzyme according to the invention involved in the pleuromutilin biosynthetic pathway. Also contemplated is a nucleic acid molecule, which may encode both an acyltransferase according to the invention and a geranylgeranyldiphosphate synthase according to the invention involved in the pleuromutilin biosynthetic pathway. Also contemplated is a nucleic acid molecule, which may encode both at least one cytochrome P450 enzyme according to the invention and a geranylgeranyldiphosphate synthase according to the invention involved in the pleuromutilin biosynthetic pathway. The isolated nucleic acid molecule may also encode a diterpene synthase according to the invention, at least one cytochrome P450 enzyme according to the invention and a geranylgeranyldiphosphate synthase according to the invention involved in the pleuromutilin biosynthetic pathway. Alternatively, the isolated nucleic acid molecule may also encode a diterpene synthase according to the invention, an acyltransferase according to the invention and a geranylgeranyldiphosphate synthase according to the invention involved in the pleuromutilin biosynthetic pathway. Alternatively, the isolated nucleic acid molecule may also encode a diterpene synthase according to the invention, an acyltransferase according to the invention and at least one cytochrome P450 enzyme according to the invention involved in the pleuromutilin biosynthetic pathway. Finally, the isolated nucleic acid molecule may also encode a diterpene synthase according to the invention, a geranylgeranyldiphosphate synthase according to the invention, an acyltransferase according to the invention and at least one cytochrome P450 enzyme according to the invention involved in the pleuromutilin biosynthetic pathway. In one embodiment (i), the at least one cytochrome P450 enzyme is a polypeptide having the amino acid sequence shown in SEQ ID NO: 41 or a polypeptide having cytochrome P450 activity and comprising an amino acid sequence having at least 60% sequence identity to SEQ ID NO: 41, as defined above. In another embodiment (ii), the at least one cytochrome P450 enzyme is a polypeptide having the amino acid sequence shown in SEQ ID NO: 47 or a polypeptide having cytochrome P450 activity and comprising an amino acid sequence having at least 60% sequence identity to SEQ ID NO: 47, as defined above. In still another embodiment (iii), the at least one cytochrome P450 enzyme is a polypeptide having the amino acid sequence shown in SEQ ID NO: 49 or a polypeptide having cytochrome P450 activity and comprising an amino acid sequence having at least 60% sequence identity to SEQ ID NO: 49, as defined above. The term “at least one cytochrome P450 enzyme” in this context means one, two, or three cytochrome P450 enzymes selected from the above embodiments (i), (ii), and (iii). Particularly preferred is a combination of (i) and (ii), or a combination of (i) and (iii), or a combination of (ii) and (iii), as well as a combination of (i), (ii) and (iii).

A third aspect concerns a vector comprising a nucleic acid molecule of the present invention. Vectors are generally known in the art. Commonly vectors include phenotypical markes, preferably selectable markers, non-limiting examples of which include markers providing antibiotic resistance or prototrophy for certain amino acids. Alternatively, the markers may be screenable markers. Also contemplated are particular expression vectors, such as tandem expression vectors or polycistronic expression vectors.

A fourth aspect relates to a host, particularly a non-naturally-occurring host selected from a cell, tissue and non-human organism, said host comprising at least one nucleic acid molecule of the invention and/or at least one vector of the invention. Particularly, said host is a fungal host, more particularly a fungus from the division basidomycota, even more particularly from the order agaricales, even more particularly from the family entolomataceae, especially from the genus Clitopilus or from the genus Pleurotus.

In some preferred embodiments, the host comprises a nucleotide sequence which nucleotide sequence comprises a sequence having at least 70% sequence identity to SEQ ID NO: 8, more preferably at least 80%, even more preferably at least 85%, or even at least 90%, such as even more preferably at least 95% sequence identity to SEQ ID NO: 8. In further preferred embodiments, the host comprises a nucleotide sequence which nucleotide sequence comprises a sequence having at least 70% sequence identity to SEQ ID NO: 40, more preferably at least 80%, even more preferably at least 85%, or even at least 90%, such as even more preferably at least 95% sequence identity to SEQ ID NO: 40. In further preferred embodiments, the host comprises a nucleotide sequence which nucleotide sequence comprises a sequence having at least 70% sequence identity to SEQ ID NO: 42, more preferably at least 80%, even more preferably at least 85%, or even at least 90%, such as even more preferably at least 95% sequence identity to SEQ ID NO: 42. In further preferred embodiments, the host comprises a nucleotide sequence which nucleotide sequence comprises a sequence having at least 70% sequence identity to SEQ ID NO: 44, more preferably at least 80%, even more preferably at least 85%, or even at least 90%, such as even more preferably at least 95% sequence identity to SEQ ID NO: 44. In further preferred embodiments, the host comprises a nucleotide sequence which nucleotide sequence comprises a sequence having at least 70% sequence identity to SEQ ID NO: 46, more preferably at least 80%, even more preferably at least 85%, or even at least 90%, such as even more preferably at least 95% sequence identity to SEQ ID NO: 46. In further preferred embodiments, the host comprises a nucleotide sequence which nucleotide sequence comprises a sequence having at least 70% sequence identity to SEQ ID NO: 48, more preferably at least 80%, even more preferably at least 85%, or even at least 90%, such as even more preferably at least 95% sequence identity to SEQ ID NO: 48. In other preferred embodiments, the host comprises a nucleotide sequence which nucleotide sequence comprises a sequence having at least 70% sequence identity to SEQ ID NO: 13, more preferably at least 80%, even more preferably at least 85%, or even at least 90%, such as even more preferably at least 95% sequence identity to SEQ ID NO: 13. In other preferred embodiments, the host comprises a nucleotide sequence which nucleotide sequence comprises a sequence having at least 50%, particularly at least 60%, especially at least 70% sequence identity to SEQ ID NO: 15, more preferably at least 80%, even more preferably at least 85%, or even at least 90%, such as even more preferably at least 95% sequence identity to SEQ ID NO: 15.

It is envisaged that based on the disclosure herein, and particularly on the disclosure of the putative DTS coding sequence (cds) and gene according to SEQ ID NOs: 8, 13, or coding sequences SEQ ID NOs: 40, 42, 44, 46, 48 as well as on SEQ ID NO: 15 including the supposed gene cluster involved in a biosynthetic pathway for producing a diterpene, a nucleic acid molecule of the invention encoding a diterpene synthase, an acyltransferase, a GGDPS, and/or a cytochrome P450 enzyme may be easily obtained by the skilled person. That is, employing bioinformatics techniques/computational techniques such as gene prediction software such as “GeneScan” a computational tool for the genome-wide prediction of protein coding genes from eukaryotic DNA sequences, may suitably be employed to identify the identity and location of said nucleic acid molecules as well as of further nucleic acid sequences encoding proteins involved in the biosynthetic pathway for producing a diterpene which sequences are part of the gene cluster described herein.

In addition, SEQ ID NO: 15, which sequence is envisaged to encode a gene cluster involved in a biosynthetic pathway for producing a diterpene, may suitably be employed in this respect. Said nucleic acid molecule may e.g. be removed from a Clitopilus passeckerianus, and/or may be modified by routine techniques such as site directed mutagenesis in order to arrive at further nucleotide sequences of the invention.

Particularly in light of the partial nucleic acid sequences SEQ ID NOs: 10 to 12, which encode conserved regions of a preferred polypeptide of the invention, and in particular SEQ ID NO: 8, 40, 42, 44, 46, 48, which encode the preferred polypeptides of the invention, and in particular SEQ ID NO: 15, which sequence is envisaged to encode a gene cluster involved in a biosynthetic pathway for producing a diterpene, the skilled person is considered to be readily in a position to identify and obtain nucleic acids of the invention. It is envisaged that based on the disclosure herein nucleic acid molecules of the invention as well as a vector of the invention may also be obtained by a procedure which is outlined below: First steps are, for example, preparing a messenger RNA population isolated from a specific cell culture, e.g. a cell culture from Clitopilus passeckerianus such as mycelium from Clitopilus passeckerianus, preparing DNA probes suitable for hybridizing with at least a part of the desired messenger RNA. Exemplary DNA probes that are considered suitable for this purpose may be based on DNA sequences such as SEQ ID NOs 8 and 10 to 13, of which DNA sequence SEQ ID NO: 8 is a nucleic acid sequences encoding a preferred polypeptide of the invention and SEQ ID NOs: 10 to 12 encode conserved regions of said preferred polypeptide of the invention. Further useful DNA probes that are considered suitable for this purpose may be based on the DNA sequences shown in SEQ ID NOs 40, 42, 44, 46, and 48, which are each a nucleic acid sequence encoding a preferred polypeptide of the invention.

Subsequent steps are screening the messenger RNA population by said DNA probes, preparing cDNA molecules from the messenger RNAs via a reverse transcriptase, cloning the cDNA molecules into vectors, analyzing these vectors, e.g. by means of restriction enzymes or DNA sequencing, and selecting vectors which carry or are likely to carry the cDNA fragments. The cDNA fragment may subsequently be transferred into a suitable expression vector, equipped with well-known genetic elements that allow expression of the polypeptide. In case the cDNA fragment does not represent the desired gene, cDNA fragments of different clones may be combined and subsequently inserted in a suitable expression vector, equipped with well-known genetic elements that allow expression of the polypeptide. Adjacent regions of a region of interest may be further analyzed by means of screening a cosmid library containing larger sequence portions, or by techniques such as genome walking, e.g. within a gene bank comprising mutually overlapping DNA regions. It is envisaged that the nucleic acid molecules may be prepared from Clitopilus passeckerianus or another organism which is known or suspected to be capable of producing a diterpene, particularly a pleuromutilin antibiotic, especially pleuromutilin. Preferably, the organism may be a fungal host, more particularly a fungus from the division basidomycota, even more particularly from the order agaricales, even more particularly from the family entolomataceae, in particular from the genus Clitopilus or Pleurotus, especially one of the pleuromutilin producers disclosed herein. Positive clones may be analyzed e.g. by restriction enzyme analysis, sequencing, sequence comparisons and others.

One exemplary suitable technique to obtain a full length sequence of an RNA transcript is known as “RACE” (rapid amplification of cDNA ends), which results in the production of a cDNA copy of the RNA sequence of interest, produced through reverse transcription of the cDNA copies. PCR-amplified cDNA copies are then sequenced. RACE may provide the sequence of an RNA transcript from a small known sequence within the transcript to the 5′ end (5′ RACE-PCR) or 3′ end (3′ RACE-PCR) of the RNA. This method is further exemplified in Example 3 below.

Tools to more closely define the borders of the gene cluster and uncover the regulation of the cluster, which are known to the skilled person, include transcription profiling by qRT-PCR (quantitative real time polymerase chain reaction) where amplified cDNA is detected in real time as the reaction progresses. In particular this technique is suitable to identify co-regulation and to quantify the expression level of genes which belong to the gene cluster and which are functional related. In addition transcription profiling can also be applied to identify functional related genes which are located elsewhere in the genome. This method is further exemplified in Example 3 below.

Suitable details for assisting the obtaining of a nucleic acid sequence of the invention on the basis of the disclosure may e.g. be taken from Kawaide et al., 1997, or Kilaru et al., 2009b.

Sequences of polypeptides of the invention may easily be deduced from the thus obtained nucleic acid sequences. Moreover, a polypeptide of the invention may be prepared by using the thus obtained cDNA (nucleic acid molecule) or expression vector in a suitable method to achieve expression of polypeptides, such as by introduction into a host.

Accordingly, a fifth aspect concerns a method of producing a polypeptide of the invention, the method comprising (i) introducing into a host selected from a cell, tissue and non-human organism at least one nucleic acid molecule of the invention and/or at least one vector of the invention, and (ii) cultivating the host under conditions suitable for the production of the polypeptide. Particularly, the method comprises a further step of (iii) recovering the polypeptide from the host.

Methods for introducing genetic material into a host are well known to the skilled person, preferred ways depending on the respective host. For example in case that the host is a fungus such as from the genus Clitopilus, non-limiting examples are considered to include Agrobacterium-mediated transformation systems and PEG-mediated transformation systems (cf. Kilaru et al., 2009b).

Methods of recovering or purifying, respectively, polypeptides from a host are known to a person skilled in the art. These methods may employ any known chromatographic techniques such as ion exchange chromatography or HPLC, centrifugation techniques such as ultracentrifugation, precipitation techniques such as ammonium sulfate precipitation, differential solubilization techniques, and the like. Conveniently, a polypeptide may be purified by any known technique involving the use of an N-terminal or a C-terminal tag, such as a His-tag, and a corresponding purification technique, such as involving Ni²⁺-affinity chromatography. Step (iii) may also be dispensable when employing means that allow secretion of the polypeptide from the host such as by using signal peptides. In the latter case, the growth medium may be used as is or may be subjected to one or more purification steps.

A sixth aspect concerns a method of producing pleuromutilin, the method comprising (i) introducing into a host selected from a cell, tissue and non-human organism a gene cluster of the invention such as a nucleic acid molecule having a sequence as defined in Ba′ above, and (ii) cultivating the host under conditions suitable for the production of pleuromutilin.

In one embodiment, said method comprises introducing a vector comprising a sequence as defined in Ba′ above in addition to or instead of a nucleic acid molecule having a sequence as defined in Ba′ above.

The host may be a host capable or incapable of producing pleuromutilin and preferably is a host incapable of producing pleuromutilin. The host may be incapable of producing pleuromutilin due to the absence of a whole gene cluster for producing pleuromutilin or due to the absence of parts thereof, which is why said method is considered feasible by providing the whole cluster, such as a sequence as defined in Ba′ above. Alternatively, only (the missing) parts of the cluster may be provided to complete the cluster and allow production of pleuromutilin. Particularly, the host may be incapable of producing pleuromutilin due to the absence of a nucleic acid encoding a pleuromutilin synthase or a polypeptide having pleuromutilin synthase activity as disclosed herein. In this case, a nucleic acid molecule or polypeptide of the invention may be introduced. Generally, more than one copy of the nucleic acid molecule may be introduced to increase production of pleuromutilin. The nucleic acid molecule(s) may be part of a vector. Upon introduction, the introduced nucleic acid molecule(s) may or may not integrate into a chromosome.

A seventh aspect generally concerns a method of altering the production of pleuromutilin in a host selected from a cell, tissue and non-human organism. In preferred embodiments, it concerns a method of altering the production of pleuromutilin in a host selected from a cell, tissue and non-human organism, wherein said host is capable of producing pleuromutilin and comprises at least one nucleic acid molecule comprising a nucleotide sequence as defined in A) or B) above, the method comprising manipulating i) the expression, ii) the identity, or iii) both the expression and the identity of said at least one nucleic acid molecule.

It is contemplated that this method is a method of increasing the production of pleuromutilin. Increasing the production of pleuromutilin may be achieved by directly or indirectly manipulating the expression of said at least one nucleic acid molecule. Direct manipulation in this respect may for example be achieved by the provision of further copies of said at least one nucleic acid molecule such as by the introduction of vectors disclosed herein and/or or by incorporation into a chromosome. In a preferred embodiment, overexpression of one or more nucleic acid molecules of the invention is achieved by means of one or more vectors. In one embodiment, the sequence of SEQ ID NO: 15 obtainable from Clitopilus passeckerianus, which sequence is envisaged to encode a gene cluster involved in a biosynthetic pathway for producing a diterpene and which sequence is obtainable by molecular biological methods known in the art, if the skilled man is provided with the information given herein, in particular the nucleic acid sequences SEQ ID NO: 8 and 10-12, is introduced into the host. Alternatively or in addition, a nucleic acid molecule of the invention comprising the nucleic acid sequences SEQ ID NO: 40, 42, 44, 46 and/or 48 may be introduced into the host. Suitable vectors and expression systems are known in the art, and include those vector/selection systems described in the following publications, which are hereby incorporated by reference, as examples for Basidomycetes:

Binninger et al., 1987; Kilaru et al., 2009a; and Kilaru et al., 2009b, and those vector/selection systems described in the following publications, which are hereby incorporated by reference, as examples for Streptomycetes: Lacalle et al., 1992; Jones and Hopwood, 1984 and Motamedi and Hutchison, 1987.

Indirect manipulation in this respect may for example be achieved by the provision of elements such as suitable promoters or enhancers, or by the removal of repressor binding sites or terminators, or by combinations thereof. Promoters, enhancers, terminators, and the like are known to the skilled person. The production of pleuromutilin may also be increased by manipulating the identity of said at least one nucleic acid molecule such as by mutagenesis thereof and selection for increased pleuromutilin production. Alternatively, the production of pleuromutilin may be increased by a combination thereof. The expression may also be indirectly increased by optimizing the expression and/or transcriptional regulation of at least one nucleic acid molecule during fermentation of the host through the adjusting of physiological parameters and/or fermentation conditions. In one embodiment, regulatory genes and/or DNA binding sites of regulatory proteins of the at least one nucleic acid molecule are influenced.

It is also contemplated that the method of the seventh aspect is a method of decreasing the production of pleuromutilin. Particularly, it may comprise disrupting or down-regulating said at least one nucleic acid molecule. Methods for direct manipulation in this respect include the targeted disruption of genes that is well-known within the art. Therefore, e.g. a mutated host such as a mutated Clitopilus strain, such as a mutated Clitopilus passeckerianus strain may be constructed, from which one or more natural nucleic acids encoding polypeptides involved in the biosynthetic pathway for producing pleuromutilin have been partly or completely deleted from the genome. Alternatively, the identity of said at least one nucleic acid molecule may be manipulated e.g. by mutagenesis thereof and selection for decreased pleuromutilin production. Generally, other suitable such methods to decrease the production of pleuromutilin may involve the use of RNA interference (RNAi). This method is further exemplified in Example 4 below. The feasibility for an RNAi mediated gene silencing as a means of knocking down expression of specific genes has been demonstrated just recently in the dikaryotic Clitopilus passeckerianus by Kilaru et al., 2009b). Here, it is believed to be more convenient than targeted gene disruption due to the dikaryotic nature of this fungus. The methods described in this reference are contemplated to be likewise applicable in other basidiomycetes, particularly in other members of the genus Clitopilus, such as the preferred ones disclosed herein including Clitopilus passeckerianus. Alternatively or additionally, production of pleuromutilin may be decreased by indirectly manipulating the expression by means of removal of elements such as suitable promoters or enhancers, or by the provision of repressor binding sites or terminators, or by combinations thereof.

Generally, e.g. (limited) mutagenesis of the pleuromutilin gene cluster in, for example, promoter regions or coding sequences, may be used for altering the production of pleuromutilin. For example, it can lead to a beneficial increase in the capabilities of a pleuromutilin producing organism to produce a pleuromutilin precursor or can even change the final product of pleuromutilin biosynthesis.

The production of pleuromutilin in a host may also be altered by replacing individual sections in the at least one nucleic acid molecule by other sections, such as sections from other gene clusters. The production of pleuromutilin in a host may also be altered by inactivating individual steps in the biosynthetic pathway for producing pleuromutilin, such as by deleting or disrupting other polypeptides of the gene cluster. Also in this aspect, the host is preferably as defined hereinabove.

The method of altering the production of pleuromutilin in a host selected from a cell, tissue and non-human organism may particularly involve the use of an isolated nucleic acid molecule, a vector or of a host cell of the invention a) for overexpressing at least one nucleic acid molecule encoding a polypeptide involved in the biosynthetic pathway for producing pleuromutilin; or b) for inactivating or modifying one or more genes involved in the biosynthetic pathway for producing pleuromutilin; or c) for constructing a non-naturally occurring host from which one or more genes involved in the biosynthetic pathway for producing pleuromutilin have been deleted; or c) for constructing a mutated host, such as Clitopilus strains, from which one or more genes involved in the biosynthetic pathway for producing pleuromutilin have been deleted or disrupted.

The method of altering the production of pleuromutilin in a host selected from a cell, tissue and non-human organism may also involve the redirection of metabolic fluxes towards the educt of the MVA pathway, i.e. acetyl-CoA, and/or to increase the amount of substrate for the geranylgeranyl disphosphate synthase, e.g. by cutting off non-essential reactions competing for the precursors IPP and DMAPP.

An eighth aspect concerns the use of a nucleic acid molecule of the invention in the production of pleuromutilin, wherein 2 to 50 nucleotides of the sequence of said nucleic acid molecule are divergent from a sequence of a gene cluster involved in the biosynthetic pathway for producing pleuromutilin comprised by a wild type organism capable of producing pleuromutilin Preferably, said 2 to 50 nucleotides are at least 3, 4, 5, 6, 7, 8, 9, 10, 11, 12, 13, 14, 15, 16, 17, 18, 19, 20, 25, 30, 35, 40, or 45 nucleotides, preferably from 3 to 100, more preferably from 5 to 80 nucleotides, even more preferably from 8 to 60 nucleotides. Such limited mutagenesis of the pleuromutilin gene cluster in, for example, promoter regions or coding sequences, can lead to a beneficial increase in the capabilities of a pleuromutilin producing organism to produce pleuromutilin or can even change the final product of pleuromutilin biosynthesis, thereby yielding a higher yield of pleuromutilin precursors. Yet, said nucleic acid molecule must not be identical to a sequence of a gene cluster involved in the biosynthetic pathway for producing pleuromutilin comprised by a wild type organism capable of producing pleuromutilin or any one of the organisms selected for pleuromutilin production as of the priority date of this application. The use is not particularly limited and may comprise any of the methods for producing pleuromutilin disclosed herein. The use may involve the complete gene cluster as disclosed above, or any partial sequence thereof as long as the sequence employed is divergent from a sequence of a gene cluster involved in the biosynthetic pathway for producing pleuromutilin comprised by a wild type organism, particularly a wild type organism capable of producing pleuromutilin. The use may e.g. be an in vivo, ex vivo or in vitro use or a combination thereof.

A ninth aspect concerns the use of an isolated nucleic acid molecule of the invention in the production of a pleuromutilin precursor, wherein 2 to 50 nucleotides of the sequence of said nucleic acid molecule are divergent from a sequence encoding a diterpene synthase comprised by a wild type organism capable of producing pleuromutilin. Preferably, said 2 to 50 nucleotides are at least 3, 4, 5, 6, 7, 8, 9, 10, 11, 12, 13, 14, 15, 16, 17, 18, 19, 20, 25, 30, 35, 40, or 45 nucleotides, preferably from 3 to 100, more preferably from 5 to 80 nucleotides, even more preferably from 8 to 60 nucleotides. Such limited mutagenesis of the pleuromutilin gene cluster in, for example, promoter regions or coding sequences, can lead to a beneficial increase in the capabilities of a pleuromutilin producing organism to produce a pleuromutilin precursor or can even change the final product of pleuromutilin biosynthesis, which may e.g. result in a higher yield of pleuromutilin precursors. Particularly, the pleuromutilin precursor is a compound according to formula (I). Alternatively, the pleuromutilin precursor may be a compound according to formula (II). In another embodiment, the pleuromutilin precursor may be a compound according to formula (III). In still another embodiment, the pleuromutilin precursor may be a compound according to formula (IV). The use is not particularly limited and may comprise any of the methods for producing a pleuromutilin precursor disclosed herein. The use may involve the complete gene cluster as disclosed above, or any partial sequence thereof as long as the sequence employed is divergent from a sequence encoding a diterpene synthase comprised by a wild type organism, particularly a wild type organism capable of producing pleuromutilin. The use may e.g. be an in vivo, ex vivo or in vitro use or a combination thereof.

A tenth aspect concerns the use of a host according to claim 8, in the production of pleuromutilin or of a pleuromutilin precursor, particularly wherein said pleuromutilin precursor is a compound according to formula (I).

Also contemplated is the use of a host according to the invention, in the production of a pleuromutilin precursor according to formula (II). Further contemplated is the use of a host according to the invention, in the production of a pleuromutilin precursor according to formula (III). Finally, the use of a host according to the invention, in the production of a pleuromutilin precursor according to formula (IV) is also contemplated.

The use is not particularly limited. It may involve any of the hosts disclosed herein and may e.g. be an in vivo, ex vivo or in vitro use or a combination thereof.

Similarly, the invention concerns methods for the production of pleuromutilin or pleuromutilin precursor that correspond to the uses of the eight to tenth aspect and include at least one step of using an isolated nucleic acid molecule, a vector, or a host of the invention.

Likewise, methods for the production of pleuromutilin or pleuromutilin precursor are also featured, which comprise the use of a polypeptide of the invention, e.g. in a solid state fermentation process.

An eleventh aspect concerns the use of an isolated nucleic acid molecule of the invention for identifying one or more nucleic acids encoding a polypeptide having diterpene synthase activity. Preferably, such nucleic acids encoding a polypeptide having diterpene synthase activity encode a polypeptide having pleuromutilin synthase activity.

Likewise, the isolated nucleic acid molecule of the invention may be used for identifying one or more nucleic acids encoding a polypeptide having acyltransferase activity. Preferably, such nucleic acids encoding a polypeptide having acyltransferase activity encode a polypeptide involved in the pleuromutilin biosynthetic pathway.

Alternatively, the isolated nucleic acid molecule of the invention may be used for identifying one or more nucleic acids encoding a polypeptide having cytochrome P450 activity. Preferably, such nucleic acids encoding a polypeptide having cytochrome P450 activity, such as monooxygenase activity, encode a polypeptide involved in the pleuromutilin biosynthetic pathway.

Finally, the isolated nucleic acid molecule of the invention may be used for identifying one or more nucleic acids encoding a polypeptide having geranylgeranyldiphosphate synthase activity. Preferably, such nucleic acids encoding a polypeptide having geranylgeranyldiphosphate synthase activity encode a polypeptide involved in the pleuromutilin biosynthetic pathway.

In addition, this aspect concerns the use of an isolated nucleic acid molecule of the invention for identifying one or more nucleic acids encoding a diterpene synthase, preferably encoding a pleuromutilin synthase.

In certain embodiments, said aspect concerns the use of a nucleic acid molecule of the invention for identifying a gene cluster of the invention involved in a biosynthetic pathway for producing a diterpene, particularly for producing pleuromutilin.

For example, the nucleic acid molecule of the invention may be used in a method which is characterized by the steps of establishing a cDNA or genomic library, screening said library by using a nucleic acid molecule disclosed herein, and isolating positive clones. That is to say, the nucleic acid molecules disclosed herein or partial sequences thereof may be employed as probes for screening a cDNA or genomic library. Various standard methods are available for identifying positive clones. In some embodiments, to improve detectability, an isolated nucleic acid molecule described herein may be labeled with a detectable label, such as any label that is easily identifiable e.g. by known physical or chemical methods. Detectable labels are well-known within the art and include, but are not limited to radiolabels, chromophores, fluorescent agents, enzymes, coenzymes, substrates, enzyme inhibitors, antibodies and the like.

The cDNA or genomic library may be prepared from any candidate organism, particularly from an organism which is known or suspected to be capable of producing a diterpene, particularly a pleuromutilin antibiotic, especially pleuromutilin. Preferably, the organism is selected from a fungal host, more particularly a fungus from the division basidomycota, even more particularly from the order agaricales, even more particularly from the family entolomataceae, in particular from the genus Clitopilus or Pleurotus, such as from the group consisting of Clitopilus scyphoides, Clitopilus prunulus, Clitopilus hobsonii, Clitopilus pseudo-pinsitus, Clitopilus pinsitus and Clitopilus passeckerianus, especially wherein said organism is Clitopilus pinsitus or Clitopilus passeckerianus. Positive clones may be analyzed e.g. by restriction enzyme analysis, sequencing, sequence comparisons and others. Adjacent regions of a region of interest may be further analyzed by means of screening a cosmid library containing larger sequence portions, or by techniques such as genome walking, e.g. within a genomic library comprising mutually overlapping DNA regions.

A method for identifying a nucleic acid i) encoding a polypeptide having diterpene synthase activity, ii) encoding a polypeptide having pleuromutilin synthase activity, iii) encoding a diterpene synthase, and/or iv) encoding a pleuromutilin synthase, may comprise a step of performing a Southern blot with chromosomal DNA of a candidate organism to detect the presence of nucleic acids having homology with nucleic acid molecules disclosed herein.

Optionally, a method for identifying a nucleic acid i) encoding a polypeptide having diterpene synthase activity, ii) encoding a polypeptide having pleuromutilin synthase activity, iii) encoding a diterpene synthase, and/or iv) encoding a pleuromutilin synthase, may comprise a preceding step of determining whether the candidate organism is capable of producing pleuromutilin by assaying for pleuromutilin production by any known method.

Likewise, also contemplated is a method for identifying a nucleic acid i) encoding a polypeptide having acyltransferase activity, and/or ii) encoding an acyltransferase, which may comprise a step of performing a Southern blot with chromosomal DNA of a candidate organism to detect the presence of nucleic acids having homology with nucleic acid molecules disclosed herein.

Further contemplated is a method for identifying a nucleic acid i) encoding a polypeptide having geranylgeranyldiphosphate synthase activity, and/or ii) encoding a geranylgeranyldiphosphate synthase, which may comprise a step of performing a Southern blot with chromosomal DNA of a candidate organism to detect the presence of nucleic acids having homology with nucleic acid molecules disclosed herein.

Also contemplated is a method for identifying a nucleic acid i) encoding a polypeptide having cytochrome P450 activity, ii) encoding a polypeptide having monooxygenase activity, and/or iii) encoding an cytochrome P450 enzyme, which may comprise a step of performing a Southern blot with chromosomal DNA of a candidate organism to detect the presence of nucleic acids having homology with nucleic acid molecules disclosed herein. Optionally, the above methods may comprise a preceding step of determining whether the candidate organism is capable of producing pleuromutilin by assaying for pleuromutilin production by any known method.

The invention further concerns the use of an isolated nucleic acid molecule of the invention for isolating one or more nucleic acids encoding a polypeptide having diterpene synthase activity and/or encoding a polypeptide having pleuromutilin synthase activity from an organism, preferably wherein the organism is as defined above.

In addition, this aspect concerns the use of an isolated nucleic acid molecule of the invention for isolating one or more nucleic acids encoding a diterpene synthase, preferably encoding a pleuromutilin synthase from an organism, preferably wherein the organism is as defined above.

Further contemplated is the use of an isolated nucleic acid molecule of the invention for isolating one or more nucleic acids encoding an acyltransferase, preferably encoding an acylatransferase from an organism, preferably wherein the organism is as defined above.

Also contemplated is the use of an isolated nucleic acid molecule of the invention for isolating one or more nucleic acids encoding a geranylgeranyldiphosphate synthase, preferably encoding an geranylgeranyldiphosphate synthase from an organism, preferably wherein the organism is as defined above.

Finally, the use of an isolated nucleic acid molecule of the invention for isolating one or more nucleic acids encoding a cytochrome P450 enzyme, such as a monooxygenase, preferably encoding a cytochrome P450 enzyme from an organism, preferably wherein the organism is as defined above, is also contemplated.

A twelfth aspect concerns a method of production of a pleuromutilin precursor, particularly of a compound according to formula (I), wherein the method is a method for the fermentative production of said precursor and comprises the steps of (i) introducing into a host selected from a cell, tissue and non-human organism at least one nucleic acid molecule of the invention and/or at least one vector of the invention, and (ii) cultivating the host under conditions suitable for the fermentative production of said precursor.

Also contemplated is a method of production of a pleuromutilin precursor, particularly of a compound according to formula (II) and/or (III), wherein the method is a method for the fermentative production of said precursor and comprises the steps of (i) introducing into a host selected from a cell, tissue and non-human organism at least one nucleic acid molecule of the invention and/or at least one vector of the invention comprising a nucleic acid sequence encoding a cytochrome P450 enzyme, such as a monooxygenase, and (ii) cultivating the host under conditions suitable for the fermentative production of said precursor.

Further contemplated is a method of production of a pleuromutilin precursor, particularly of a compound according to formula (IV), wherein the method is a method for the fermentative production of said precursor and comprises the steps of (i) introducing into a host selected from a cell, tissue and non-human organism at least one nucleic acid molecule of the invention and/or at least one vector of the invention comprising a nucleic acid sequence encoding an acyltransferase, and (ii) cultivating the host under conditions suitable for the fermentative production of said precursor.

A thirteenth aspect concerns a method of the production of a pleuromutilin precursor, particularly of a compound according to formula (I), wherein the method is a method for the synthetic production of said precursor and comprises reacting geranylgeranylpyrophosphate with a polypeptide according of the invention or a polypeptide obtainable by a method described above. In the latter two aspects, the host is preferably as disclosed hereinabove.

In an even further aspect, the invention concerns an isolated compound according to formula (I). In some embodiments, the isolated compound according to formula (I) is obtainable by a method disclosed herein, particularly by a method of the twelfth or thirteenth aspect.

In further aspects, the description features an isolated compound according to formula (II) obtainable by a method disclosed herein; an isolated compound according to formula (III) obtainable by a method disclosed herein; and/or an isolated compound according to formula (IV) obtainable by a method disclosed herein.

In a still further aspect, the diterpene, particularly the pleuromutilin precursor, produced in accordance with the invention may be converted into a pleuromutilin antibiotic such as a new pleuromutilin antibiotic. This may be done by synthetic organic chemistry, e.g. in analogous ways to how pleuromutilin is converted to tiamulin (analogous to the process described in IN 2005CH00521), valnemulin (analogous to the process described in CN 101318921), retapamulin (analogous to the process described in WO 2009075776) and the like. The present invention thus also relates to the use of intermediate I (i.e. the compound according to formula (I)) in the semisynthetic production of a pleuromutilin antibiotic. “Semisynthetic production” relates to a combination of fermentative production of a pleuromutilin precursor followed by at least one synthetic chemical step to yield a covalent modification of the pleuromutilin precursor.

Thus the invention also concerns a method for the production of a pleuromutilin antibiotic, wherein the method comprises at least one step of reacting a diterpene or pleuromutilin precursor obtained by means of a polypeptide or method of the invention.

In an even further aspect, the new pleuromutilin antibiotic, especially the new pleuromutilin obtained in accordance with the invention is used as a medicament. In one embodiment, it is used in a method of treating a bacterial infection. Also envisaged is a method of treating a bacterial infection involving a step of administering a diterpene, particularly the new pleuromutilin antibiotic, especially the new semisynthetic pleuromutilin obtained in accordance with the invention, to a subject in need thereof, particularly a subject suffering from a bacterial infection or a disorder or disease involving a bacterium

The bacterium (causing the bacterial infection or involved in said disorder or disease) may be selected from the group consisting of Gram-positive bacteria particularly staphylococci, streptococci, pneumococci and enterococci; Gram-negative bacteria particularly selected from the genera Neisseria, Haemophilus, Moraxella, Bordetella, Legionella, Leptospira; mycoplasmas; chlamydia; Gram-positive anaerobes and Gram-negative anaerobes.

Generally, all documents cited herein are incorporated by reference herein in their entirety. Also, while certain aspects and embodiments of this invention are described or exemplified herein, it will be understood by a person skilled in the art that various modifications can be made without departing from the spirit and scope of the invention.

### EXEMPLIFYING SECTION

The following examples are meant to further illustrate, but not limit, the invention. The examples comprise technical features and it will be appreciated that the invention relates also to combinations of the technical features presented in this exemplifying section.

### Example 1

### Identification of a Diterpene Synthase (DS)

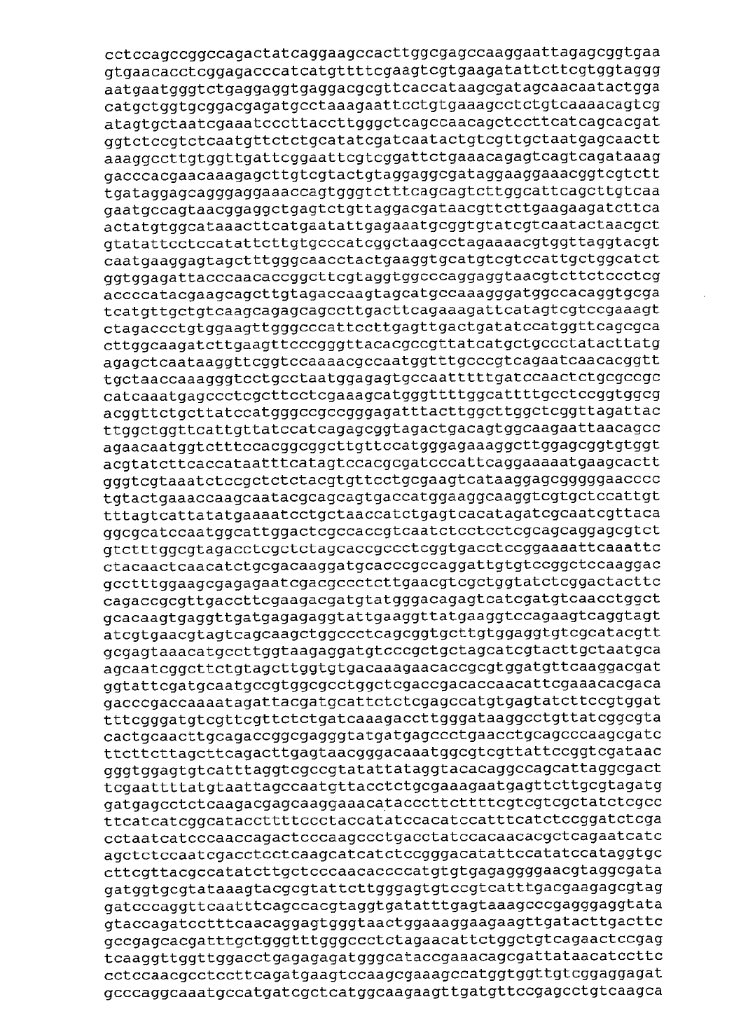

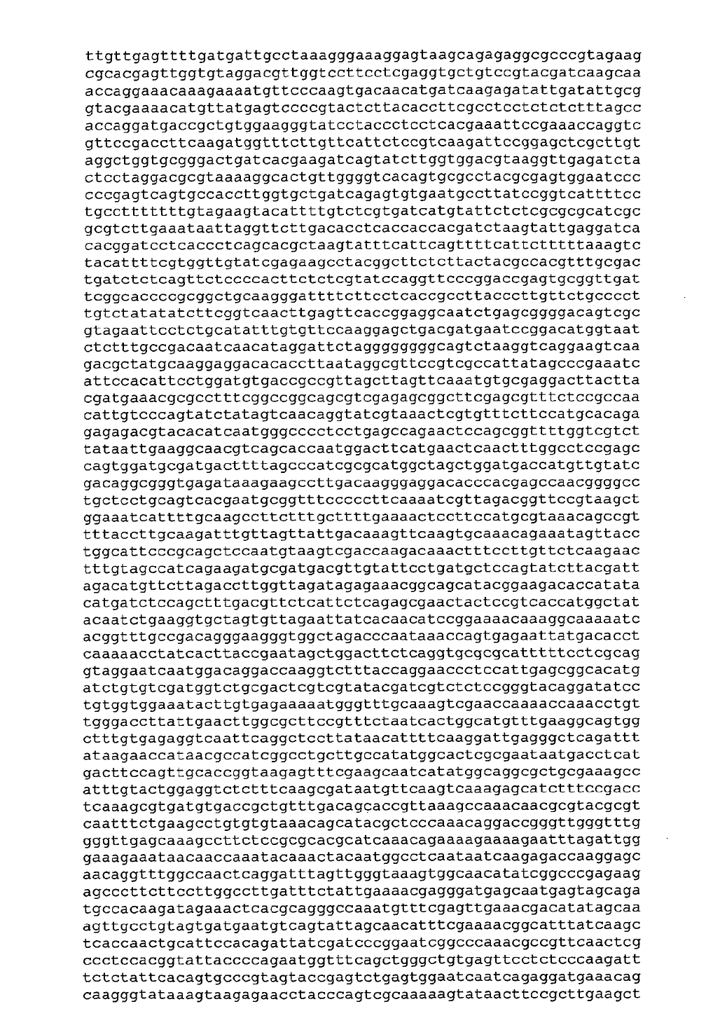

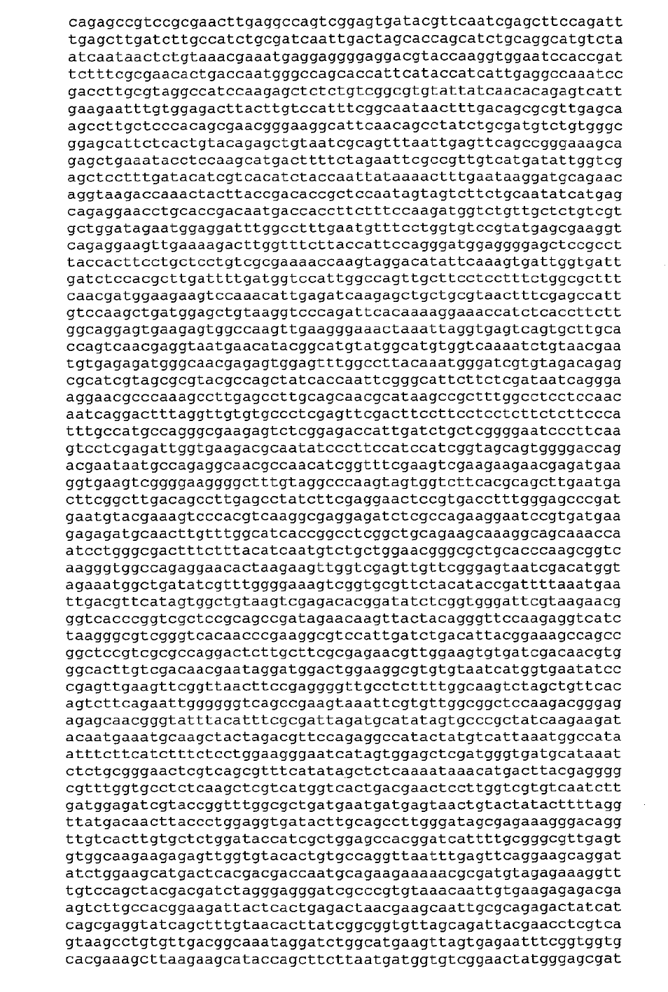

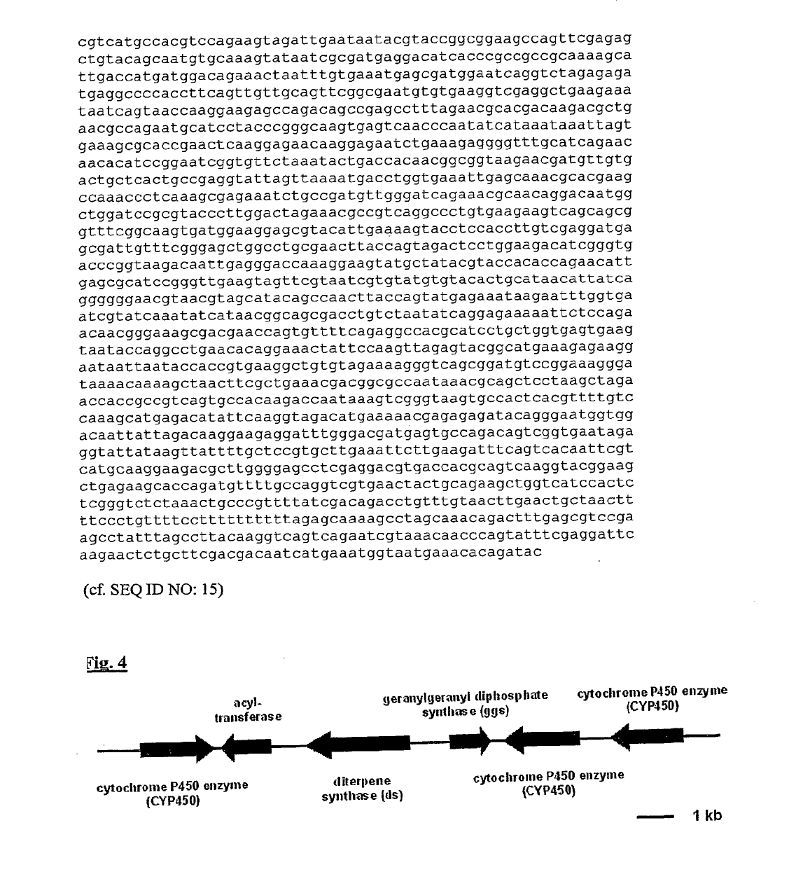

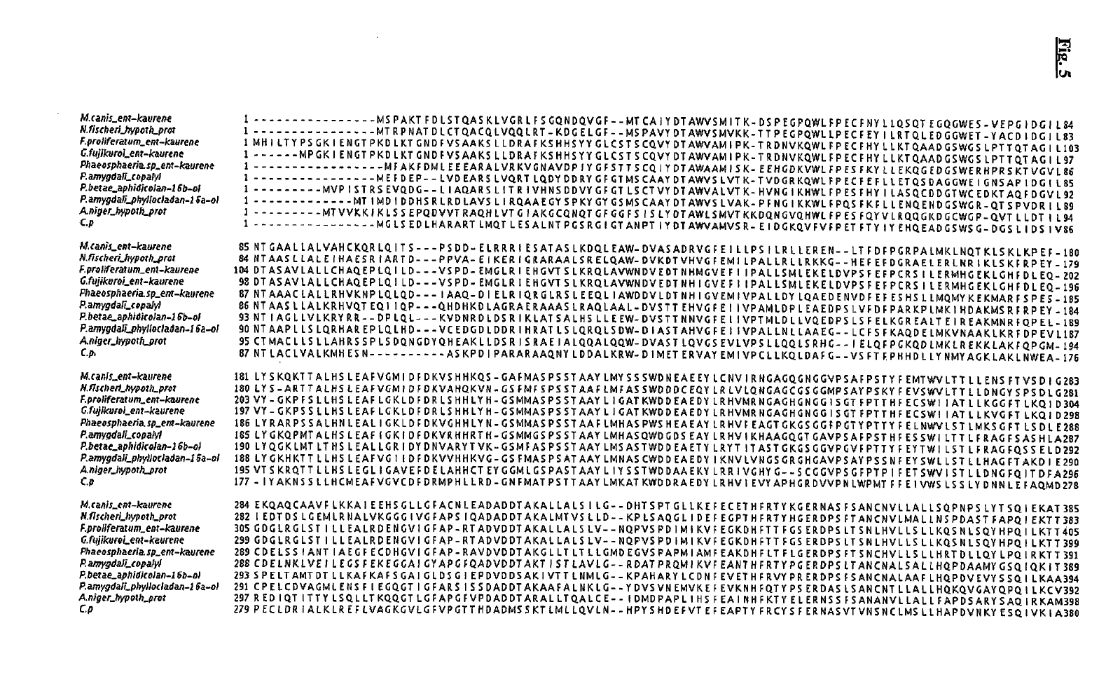

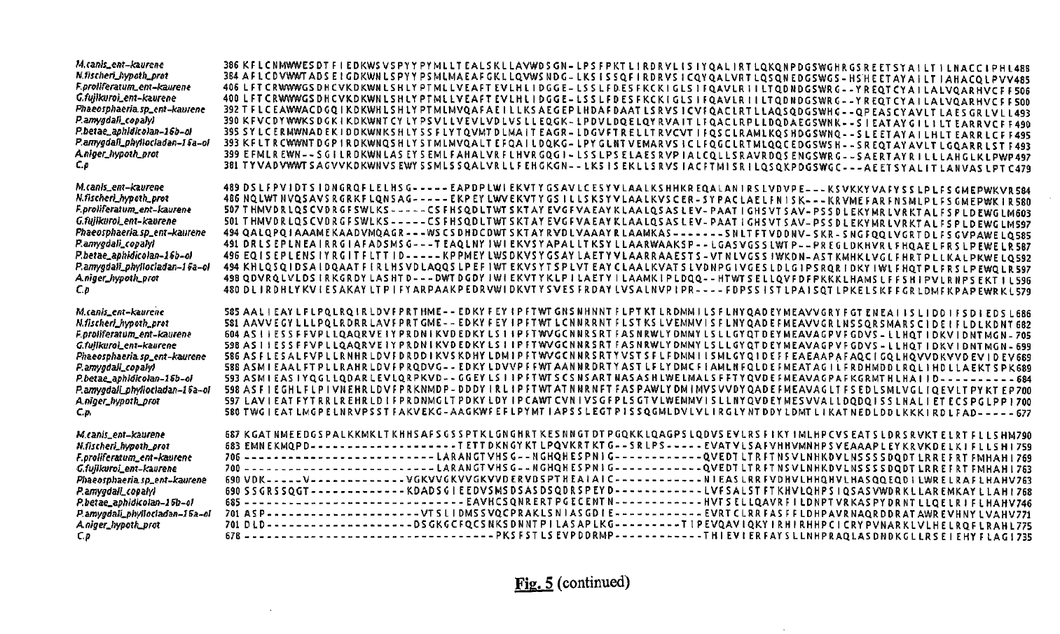

The present inventors have identified a ds gene in Clitopilus passeckerianus. Partial protein sequences of the polypeptide encoded by the ds gene that have been identified and are included herein as SEQ ID NOs: 1-7. The putative protein sequence of the diterpene synthase encoded by the ds gene is herein included as SEQ ID NO: 9 and was obtained by computational methods. Alignment of the sequence with known terpene synthase sequences has revealed numerous shared conserved regions rendering it likely that the polypeptide has diterpene synthase activity. Particularly since Clitopilus passeckerianus is known to the present inventors as a pleuromutilin producing strain and in view of the fact that this is the only diterpene synthase in Clitopilus passeckerianus, it is envisaged that the identified diterpene synthase has pleuromutilin synthase activity. An alignment of the putative protein sequence of the diterpene synthase encoded by the ds gene (designated as “C.p”) and several known diterpene synthase sequences is shown in FIG. 5.

### Example 2

### Identification of the Putative Pleuromutilin Gene Cluster in Clitopilus passeckerianus

To arrive at the present invention, the inventors have analyzed the genomic region around the putative diterpene synthase described above and have employed bioinformatics tools using genomic sequences of Clitopilus passeckerianus.

The present inventors have, in close proximity to the putative ds gene, identified a previously unknown putative geranylgeranyl diphosphate synthase (ggs) gene of Clitopilus passeckerianus. The new ggs gene from Clitopilus passeckerianus shows a close relationship to known ggs genes. An alignment with a known ggs gene of Phomopsis amygdali (BAG30959) gives 31% identity. The identities were calculated by aligning sequences with the freeware program ClustalX (Version 1.83) with default parameters and subsequent counting of identical residues by hand. Percentage identity (PID) was then calculated by dividing the number of identities by length of the shortest sequence. Default settings for, e.g., pairwise alignment (slow-accurate) are: gap opening parameter: 10.00; gap extension parameter 0.10; Protein weight matrix: Gonnet 250; DNA weight matrix IUB. The ClustalX program is described in detail in Thompson et al., 1997

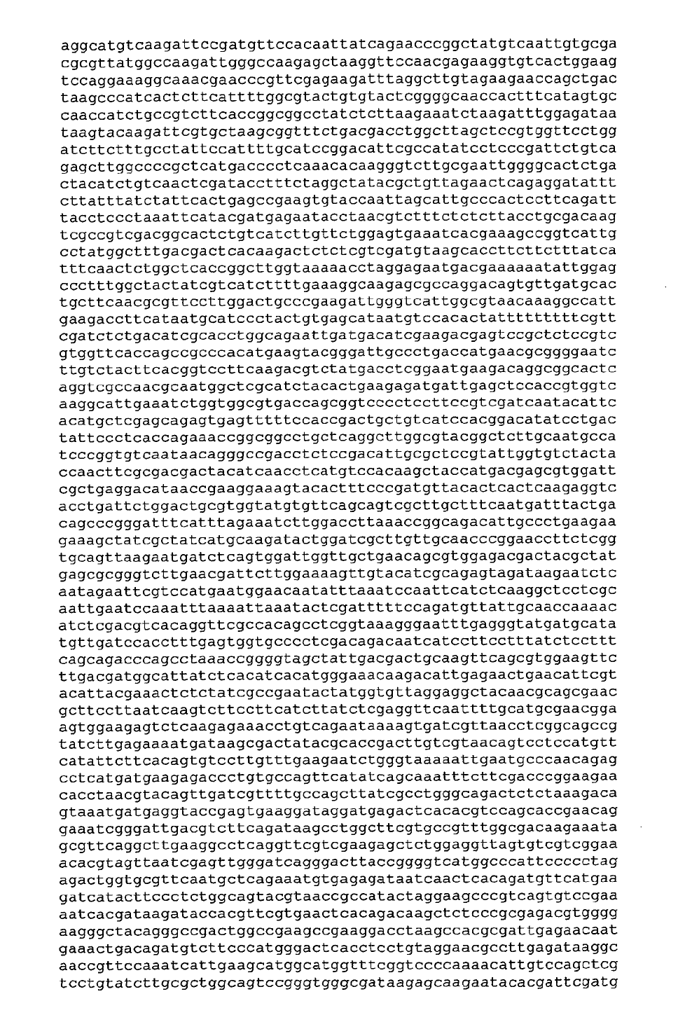

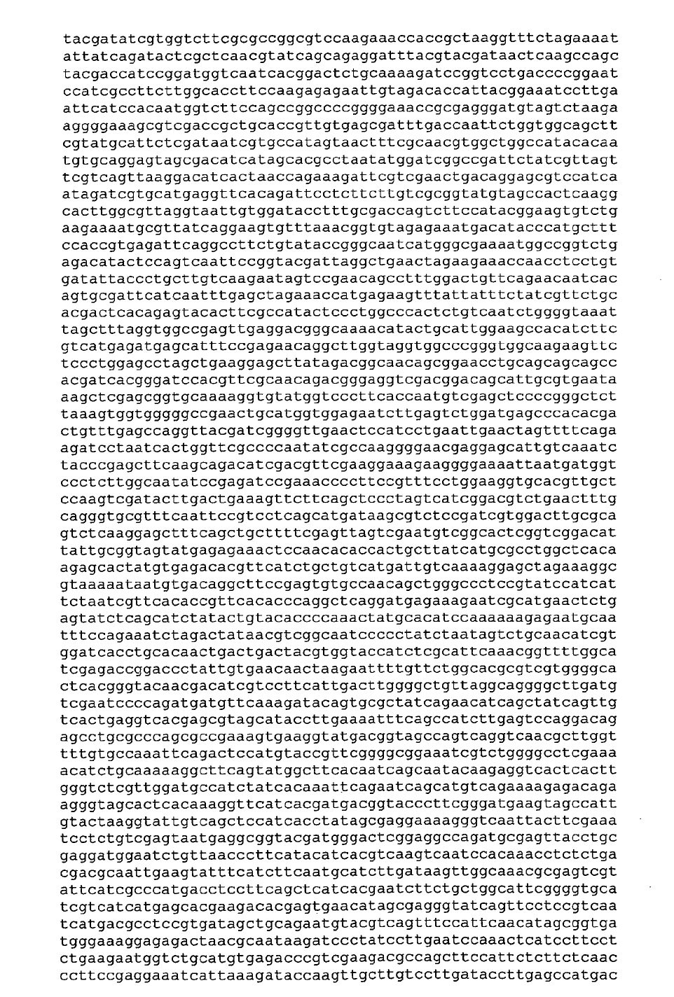

As illustrated in FIG. 4, further analysis of a genomic region comprising SEQ ID NO: 8 and having a size of about 27 kb further revealed three putative CYP450 genes, a putative acyltransferase gene as well as further putative genes in proximity to the ggs and ds genes, which suggests that this cluster is the pleuromutilin biosynthesis gene cluster. Moreover, the presence of these enzymatic activities is in line with the predicted biosynthetic pathway. Furthermore, expression analysis reveals that those genes show co-regulation under various conditions known to influence gene expression patterns, further corroborating the above conclusion.

### Example 3

### Identification of Pleuromutilin Candidate Genes in Clitopilus passeckerianus

Draft gene models for pleuromutilin candidate genes of Clitopilus passeckerianus were developed manually by blastx searching protein databases (e.g., nr=non-redundant protein sequences) using translated nucleotide queries derived from SEQ ID NO: 15. Based on these draft gene model specific primers were designed for rapid amplification of 5′- and 3′-cDNA ends (RACE). Primers (see tables below) were designed in a way that the fragments overlap, thereby obtaining full length cDNAs. RACE was done using the GeneRacer® RACE Ready cDNA Kit (Invitrogen) according to the manufacturers protocol. Amplicons were purified via agarose gels and cloned into either pCR®-Blunt II-TOPO® (Invitrogen) or pCR®4 BluntTOPO (Invitrogen).

| | | nested | |
|---|---|---|---|
| primer | sequence (5′→3′) | primer | sequence (5′→3′) |
| cyp450- | CAATGACCTATGGGCTCGAGAC | cyp450-1_3n | TCTGAGATTATGACATCTGGCG |
| 1_3 | TGAA | (27 bp) | CCTTT |
| (26 bp) | (SEQ ID NO: 16) | | (SEQ ID NO: 17) |
| cyp450- | GTTGAGGTATGGGAAAGATGG | cyp450-1_5n | GTGCCCAAGGCGGATGCAGTCG |
| 1_5 | GAAGTC | (23 bp) | T |
| (27 bp) | (SEQ ID NO: 18) | | (SEQ ID NO: 19) |
| predP-1_3 | CTGGAATTGGGAGCCGAAGATT | predP-1_3n | CGTCACAGGTTTTCGGCATTAC |
| (23 bp) | T | (26 bp) | CTTA |
| | (SEQ ID NO: 20) | | (SEQ ID NO: 22) |
| predP-1_5 | GAGAACCCCATCCTCCATCTGT | predP-1_5n | CGAGAGGAAGAATGCGGTGTA |
| (27 bp) | ATGAT | (25 bp) | CAGT |
| | (SEQ ID NO: 21) | | (SEQ ID NO: 23) |
| dts_3 | CCCATGACGAATTCGTTACAGA | dts_3n | CTGATGTCAACAAGTACGAATC |
| (26 bp) | GTTT | (27 bp) | CCAAA |
| | (SEQ ID NO: 24) | | (SEQ ID NO: 26) |
| dts_5 | CTTCGCGGATTCAATGACTTTG | dts_5n | TCGGGCTTCTGGCTCTGGAGAA |
| (26 bp) | TACA | (23 bp) | T |
| | (SEQ ID NO: 25) | | (SEQ ID NO: 27) |
| ggdps_3 | AGTCCGCTCTCCGTCGTGGTTC | ggdps_3n | CAAGACGTCTATGACCTCGGAA |
| (23 bp) | A | (26 bp) | TGAA |
| | (SEQ ID NO: 28) | | (SEQ ID NO: 30) |
| ggdps_5 | AGCTTGTGGACATGAGGTTGAT | ggdps_5n | GAGCCGTACGCCAAGCCTGAGC |
| (27 bp) | GTAGT | (23 bp) | A |
| | (SEQ ID NO: 29) | | (SEQ ID NO: 31) |
| cyp450- | TTCTTAGACTACATCCCTCGCG | cyp450-2_3n | ATTCCGGGGTCAGGACCGGAT |
| 2_3 | GTTT | (23 bp) | CT |
| (26 bp) | (SEQ ID NO: 32) | | (SEQ ID NO: 34) |
| cyp450- | CAACCGTTCCAAATCATTGAAG | cyp450-2_5n | CGATTCGATGTACGATATCGTG |
| 2_5 | CAT | (27 bp) | GTCTT |
| (25 bp) | (SEQ ID NO: 33) | | (SEQ ID NO: 35) |
| cyp450- | GCGTCATGATTGACGGAGGAAC | cyp450-3_3n | GGCGATGAATACGACTCGccmr |
| 3_3 | T | (23 bp) | T |
| (23 bp) | (SEQ ID NO: 36) | | (SEQ ID NO: 38) |
| cyp450- | CAGCCATCTTGAGTCCAGGACA | cyp450-3_5n | CATGTACCGTTCGGGGCGGAA |
| 3_5 | GA | | AT |
| (24 bp) | (SEQ ID NO: 37) | | (SEQ ID NO: 39) |

Overlapping sequences were assembled, the largest open reading frame (ORF) within each cDNA (as obtained from RACE) was translated to deduce the corresponding amino acid sequence.

The presumptive pleuromutilin core cluster encodes all the enzymes required to carry out the biochemical reactions postulated for pleuromutilin biosynthesis (I) a diterpene synthase (DTS) linked to a geranylgeranyldiphosphate synthase (GGDPS), sharing the same promoter region but transcribing the genes from complementary promoters in opposite direction (II) three cytochrome P450 (CYP450) enzymes for adding oxygen functions to the tricyclic diterpene hydrocarbon intermediate (III) an acetyltransferase (AT) for the acetylation of C14. The ORFs and the deduced amino acid sequences are shown in FIGS. 3 and 6-10. The ORFs are encoded on the putative pleuromutilin gene cluster shown in SEQ ID NO: 15 at the following positions (stop codons included):
CYP450-1: (501 . . . 699, 759 . . . 867, 917 . . . 943, 998 . . . 1186, 1240 . . . 1504, 1560 . . . 1677, 1732 . . . 1909, 1966 . . . 2070, 2120 . . . 2251, 2303 . . . 2446, 2502 . . . 2607)
AT: (2856 . . . 3281, 3335 . . . 3362, 3416 . . . 3474, 3536 . . . 4156)
DTS: (5029 . . . 6714, 6767 . . . 7053, 7109 . . . 7229, 7284 . . . 8069)
GGDPS: (9021 . . . 9158, 9217 . . . 9386, 9446 . . . 9735, 9795 . . . 10040, 10101 . . . 10309)
CYP450-2: (10725 . . . 10823, 10894 . . . 10998, 11053 . . . 11091, 11143 . . . 11258, 11316 . . . 11336, 11388 . . . 11436, 11494 . . . 11556, 11613 . . . 11811, 11865 . . . 12229, 12287 . . . 12417, 12472 . . . 12537, 12594 . . . 12681, 12730 . . . 12769, 12820 . . . 13010)
CYP450-3: (13901 . . . 13993, 14046 . . . 14135, 14187 . . . 14344, 14399 . . . 14419, 14475 . . . 14523, 14574 . . . 14636, 14693 . . . 14900, 14960 . . . 15333, 15385 . . . 15515, 15568 . . . 15721, 15778 . . . 15817, 15870 . . . 16129)

### Quantitative PCR Expression Analysis

One can assume that an increase of pleuromutilin productivity correlates with an enhanced transcription of the genes within the pleuromutilin biosynthesis cluster. Therefore the expression profiles of two strains, Clitopilus passeckerianus DSM1602 (ATCC34646, NRLL3100) and a derivative (Cp24, selected for increased pleuromutilin productivity) were analyzed. Both strains were cultivated in shake flasks, essentially as e.g. described in Hartley et al. (2009). One pre-culture was used to inoculate three parallel main-cultures (biological replicates). Two cultures each were sampled for RNA; the third one was used for pleuromutilin analytics. Samples were taken at t=72 h, 96 h, 120 h, 144 h, and 168 h of the main cultures.

Total RNA was isolated from mycelia collected by filtration on sterile cloth. The wet mycelium was flash-frozen in liquid nitrogen and ground to a powder using a mortar and pestle. Total RNA was extracted using the TRIZOL® Reagent (Invitrogen) extraction protocol. All procedures were performed according to the manufacturers protocols. After extraction the RNA samples were purified with RNeasy® columns (Qiagen) and an on-column DNase digest was performed using the RNase-Free DNase Set Kit (Qiagen) according to the manufacturers protocol. RNA was re-suspended in DEPC-treated, sterile, destilled water and its concentration was measured by spectrophotometry (Ultrospec 3100 pro, Amersham). The quality of the RNA was checked by Bioanalyzer-measurements (Agilent) using the RNA 6000 Nano Assay (Agilent). Subsequent reverse transcription was performed using the High Capacity cDNA Archive Kit (Applied Biosystems) according to the manufacturers protocol. The RT-PCR was performed with Applied Biosystems 7900HT Fast Real-Time PCR System and Power SYBR® Green PCR Mix (Applied Biosystems). All gene-specific primers were designed using Primer Express software (Applied Biosystems). Relative standard curves were prepared for each primer pair using serial dilutions of reverse-transcribed total RNA. Primers have to comply with the following criteria: −3.60≦slope≦3.10, and 0.960≦R2.

Primers used for quantitative RT-PCR analysis of Clitopilus passeckerianus:

| primer | sequence (5′→3′) |
|---|---|
| Cp_act_U1 | TGATGGTCAAGTTATCACGATTGG |
| | (SEQ ID NO: 50) |
| Cp_act_L1 | GAGTTGTAAGTGGTTTCGTGAATACC |
| | (SEQ ID NO: 51) |
| Cp_cyp450-1_U1 | TCGGCTCTACAACGCTTTCA |
| | (SEQ ID NO: 52) |
| Cp_cyp450-1_L1 | TGTCATAATCTCAGACGCTGCAA |
| | (SEQ ID NO: 53) |
| Cp_predP-1_U1 | AAGATTTTCGTCCACAGGTTCAC |
| | (SEQ ID NO: 54) |
| Cp_predP-l_L1 | TACAGCGAGACCAGATCACAAATAA |
| | (SEQ ID NO: 55) |
| Cp_dts_U1 | GTTACAGAGTTTGAGGCACCTACCT |
| | (SEQ ID NO: 56) |
| Cp_dts_L1 | CGTGGAGGAGCGACATAAGG |
| | (SEQ ID NO: 57) |
| Cp_ggdps_U1 | GACATCGAAGACGAGTCCGC |
| | (SEQ ID NO: 58) |
| Cp_ggdps_L1 | TTGAAGGACCGTGAAGTAGACAAG |
| | (SEQ ID NO: 59) |
| Cp_cyp450-2_U1 | TACATCCCTCGCGGTTTCC |
| | (SEQ ID NO: 60) |
| Cp_cyp450-2_L1 | GGTCTTCCAGCCG |
| | (SEQ ID NO: 61) |
| Cp_cyp450-3_U1 | GTCATGATTGACGGAGGAACTG |
| | (SEQ ID NO: 62) |
| Cp_cyp450-3_L1 | TCCTTCAGCTCATCACGAATCTT |
| | (SEQ ID NO: 63) |

The reaction was performed according to the manufacturers protocol using 20 μl reactions which each contained 50 ng of template cDNA. A no template control (NTC), with no added template RNA to control for any contaminants in reagents for each template is included.

The PCR conditions were: initial cycle of denaturation (10 min at 95° C.) followed by 40 cycles (15 s, 95° C. and 1 min, 60° C.). Samples were tested for nonspecific amplifications by dissociation curve determination with an additional cycle for 15 s at 95° C., 15 s at 60° C. and 15 s at 95° C. Data analysis was done with the bundled SDS software, ver. 2.3 (Applied Biosystems) using the comparative CT method.

All samples were measured in triplicates. Relative transcript level values for pleuromutilin biosynthesis genes were obtained after normalization of values calculated for the target genes (detector) against those of the beta actin gene as endogenous control.

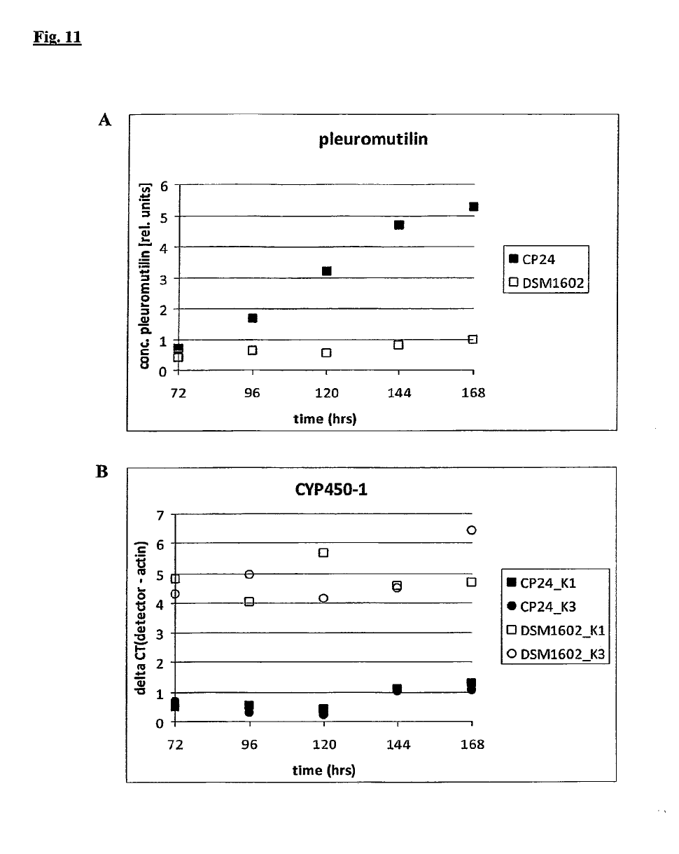

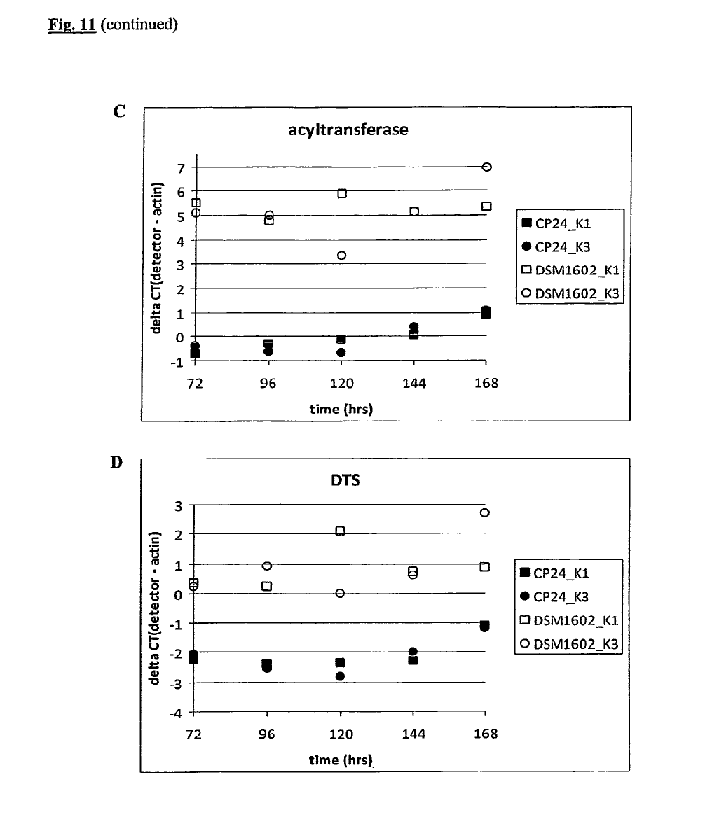

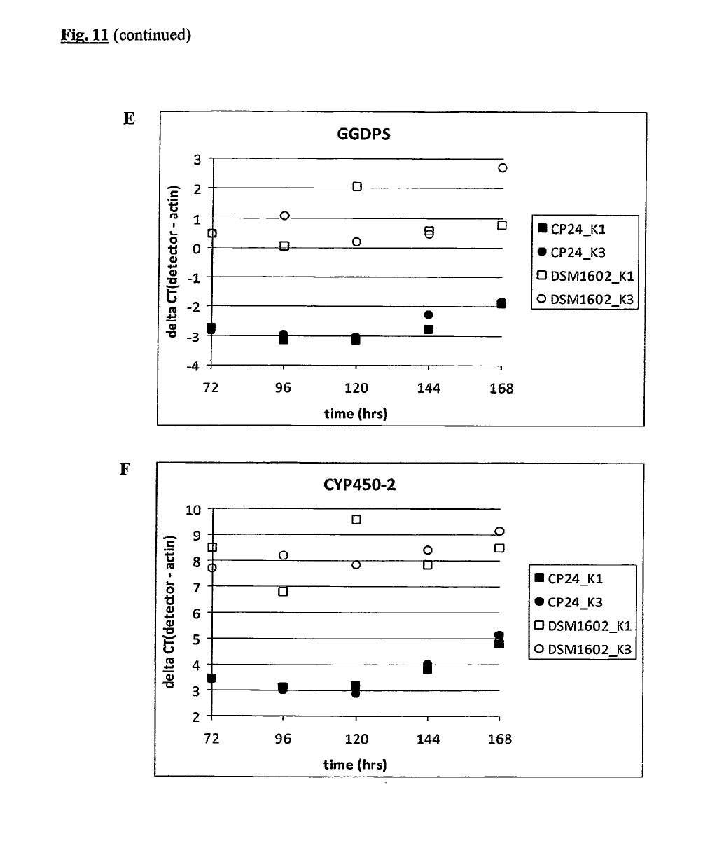

As it can be taken from the results shown in FIG. 11, core pleuromutilin cluster genes (CYP450-1, AT, DTS, GGDPS, CYP450-2, CYP450-3) show exactly the expected profiles: constitutive upregulation in Cp24. In contrast thereto, GAPDH as the negative control shows no significant differences in the expression profile.

### Example 4

### RNAi Knockdown of the Diterpene Synthase of Clitopilus passeckerianus

### Construction of Plasmids Used for RNAi

It is well known from literature that transcriptional suppression of a target gene by RNA-interference may experimentally be induced by supplying a cell with pieces of double stranded RNA partially identical with the target gene's mRNA. An efficient way to achieve this in fungi consists of transforming a cell with a plasmid transcribing a mRNA which is able to fold into a hairpin looped structure. Hairpin looped single strand nucleic acids consist of a basepaired stem structure and a spacer sequence with unpaired bases forming a loop.

Technically generating a RNAi cassette is achieved by cloning a sequence stretch and its self-complementary counterpart as repeat separated by a short spacer sequence. This RNAi cassette is positioned between a promoter and a terminator on a selection plasmid resulting in a RNAi hairpin vector, which constitutes a RNAi vector targeted against expression of the diterpene synthase gene, e.g. P2543_Hairpin.

The RNAi cassette of P2543_Hairpin is defined as continuous stretch of sequences A, B and C:

A) subsequence of the diteipene synthase gene
(forward sequence)
(SEQ ID NO: 64)
TCGCCCTCGTCTTCGCCCTTTGTCTTCTTGGTCATCAGATCAATGAAGAA
CGAGGCTCTCGCGATTTGGTGGACGTTTTCCCCTCCCCAGTCCTGAAGTA
CTTGTTCAACGACTGTGTCATGCACTTTGGTACATTCTCAAGGCTCGCCA
ACGACCTTCACAGTATCTCCCGCGACTTCAACGAAGTCAATCTCAACTCC
ATCATGTTCTCCGAATTCACCGGACCAAAGTCTGGTACCGATACAGAGAA
GGCTCGTGAAGCTGCTCTGCTTGAATTGACCAAATTCGAACGCAAGGCTA
CCGACGATGGTTTCGAGTACTTGGTCCAGCAACTCACTCCACATGTCGGG
GCCAAACGCGCACGGGATTATATCAATATAATCCGCGTCACCTACCTGCA
B) spacer containing Intron 1 (underlined) of
Cutinase gene from Magnaporthe grisea
(SEQ ID NO: 65)
ctcgaggtacgtacaagcttgctggaggatacaggtgagcGTGAGCCTTT
CTTCTTGCCTCTCTTTGTTTTTTTTTTGTTCTTTTTGCCGAATAGTGTAC
CCACTGGAGATTTGTTGGCCATGCAAATAAATGGAAGGGACTGACAAGAT
TGTGAAATTGTTCAAAACACACAGcacacagccagggaacggcagatctt
cgcatgctaaggcctcccagcccatagtcttcttctgcat
C) subsequence of the diterpene synthase gene
(reversed complement)
(SEQ ID NO: 66)
TGCAGGTAGGTGACGCGGATTATATTGATATAATCCCGTGCGCGTTTGGC
CCCGACATGTGGAGTGAGTTGCTGGACCAAGTACTCGAAACCATCGTCGG
TAGCCTTGCGTTCGAATTTGGTCAATTCAAGCAGAGCAGCTTCACGAGCC
TTCTCTGTATCGGTACCAGACTTTGGTCCGGTGAATTCGGAGAACATGAT
GGAGTTGAGATTGACTTCGTTGAAGTCGCGGGAGATACTGTGAAGGTCGT
TGGCGAGCCTTGAGAATGTACCAAAGTGCATGACACAGTCGTTGAACAAG
TACTTCAGGACTGGGGAGGGGAAAACGTCCACCAAATCGCGAGAGCCTCG
TTCTTCATTGATCTGATGACCAAGAAGACAAAGGGCGAAGACGAGGGCGA

Upon transcription of the RNAi cassette the resulting mRNA may form a hairpin structure with sequences A) and C) forming a stein and spacer B) forming a loop. Often in literature spacer sequence do contain intron sequences. The exact function of introns in the respect of RNAi induction is not understood but thought to increase RNAi efficiency. For reasons of consistency with literature intron 1 of the cutinase gene of Magnaporthe grisea has been used as part of the spacer region.

Promotor (D) and Terminator (E) have been used for efficient transcription of the hairpin cassette:

D) promoter sequence
(SEQ ID NO: 67)
gcacgcaattaagtatgttcgtcctgcggtagaaggttttcaagtagacg
tacttcgtaggatcatccgggtattttgacctcaagtcttggttcttgtt
cacggcccgttcaaatttcagaagtgttctccgtatggagggagctgaaa
gttcttcagcctgcgaagggtgagcatccaagttagttcgaggccactat
acgacactcacatcttcctgcactccttccccagcagcattctcaaatat
cttgaggatatccttttgctccgacgttaacccccctccgtagaaccgac
cctcttcatcttcttccgcgaagtagtctgcatcaccacctggcgcgaag
tctccagcatcttcatccggcacgtcttcaacgcgggcagcgcgtctttg
tctgctgccctcaggcggctctccattcatttcaacatccatactgggac
cagcagcagcacttccattgtcaagcttcatcttcttcaacatttcagga
gtgggattatccggtagcttcctcttgttcccagtcaaaggaacttttgg
gaccttgagggacacgtcaaaccttcaataactttagcttagaagcagtc
tttactgactttgaatagactgtcgatatccattggtagtcctcagtggt
tggtcgaacagaatgtggcaagcaaagtagcaaacgtgtttacgtaatgt
aatgaattcgttcatagccccctcaacagctcgtacacacaggacatggc
tcaaattcagatgtattatggtactttcaacacacagaacgccacatatg
cttaccagaagcgacaacttagggagtaaaatcctgaagttcatgaaacc
ctcaaagtgtcaatcatcattgttcaagcacatctaagcaaggcctcaca
ttatacagcagcgatagcgtaacgttgtctgaagtccttctaatatgcct
gaaaagtttagtagggctttttgcgattcttcttcaactcctgctcgagt
tgcctggcctttctgtggccaatctccacaggccggatggcagtgctgtc
tgctttcttcagtttaatgggtcggttgccgacatatttacctgaaagta
tcatcagtgagcgtagcaaaaaagaaaggtcaatgcttaccatccatctc
cttccatgccttcaagaaatcttcaggatcggcgaatgcaacgaaaccgt
actttgcctaataatagttggtaagtcgatgttgaatggaatatgagaga
ttgtccatttacctttccactgagccggtcacggataacacgcgctttct
ggaaggagacatacttgttgaaggcatttgaaaggacgtcgtcagaaacg
tcgttgctaagatcgccaacaaacaaacggaaccatgctagagaacggta
atgtcatataaatggatgcaatgtaagaatcggagagaaacacacatgga
ttccactccagcagcgtctggtcctcccaaacttttcctgctcccttcct
cagaactgtggttctctttccaccctttgcaagtttgcctccagcccctc
cacgcttgtctatcgcagccccgggaacgtaaacactttgctgagcgagt
atcccgacatcgtattcgtaagcattggcgggcacaggcgcggaatgcga
ggatgaagcaaccgggaaagaggggccttgataaggtttgtagtaaggat
taatatcctgcccttgcgacgtctgctgctgttggtattgctgataataa
ttctgactataatccatctataccgacctgaatgaacgtcgtcgaagtga
aagaaaatgcggagaaacgggatgatggcagtctgcagtcaagcactgca
acaagcctgcacagacggcagtgctgctgactcagcatacgcttatgtaa
tcccctctgtgaacagagaatctgtgtagatcgacgagggcaacacggtc
gccgtcctcaaaaccctcctccctcaaggtatgttaccgttacaaacgat
tgaaagccattctgtatgctgcgcgaatgtatcccagttgaattggagcg
aaatctgcagtattcaggatggatgcacattctcggatttggatgtcaac
gcaaaagtactgacatatcgtgatag
E) terminator sequence
(SEQ ID NO: 68)
tttgctacttcactctcaccttcacgcactttctttcatgtaccatgagc
atatgtcgatatggatatcacaccaaaatgcattcaactatgctggccaa
aaaacatgcatcacgaacgggatattatttaaccttggctgccgccaaaa
ctatactcttgacccaagcaagcaagcctacagacttgtcgccggaa

A DNA sequence containing promoter (D), RNAi cassette (A, B, C) and terminator (E) as continuous stretch has been generated by gene synthesis and cloned into selection plasmid pPHT1 (Cummings et al., 1999). The resulting plasmid P2543 Hairpin is able to transform Clitopilus passeckerianus to Hygromycin resistance and induce RNAi interference targeted against the diterpene synthase gene.

As negative control for the RNAi induction process Plasmid P2558 has been deviced. P2558 was constructed from P2543_Hairpin by replacing the RNAi cassette (AscI pos. 8′1335 to Pad pos. 1) with sequence (F) using AscI and Pad restriction sites flanking the diterpene synthase sequence F):

F) subsequence of the diterpen synthase gene
(used for construction of the negative control)
(SEQ ID NO: 69)
gggcaaccttaaatccatatccgagaagctcctgtctagggtgtccatcg
cctgcttcacgatgatcagtcgtattctccagagccagaagcccgatggc
tcttggggatgcgctgaagaaacctcatacgctctcattacactcgccaa
cgtcgcttctcttcccacttgcgacctcatccgcgaccacctgtacaaag
tcattgaatccgcgaaggcatacctcacccccatcttctacgcccgccct
gctgccaaaccggaggaccgtgtctggattgacaaggttacatacagcgt
cgagtcattccgcgatgcctaccttgtttctgctctcaacgtacccatcc
cccgcttcgatccatcttccatcagcactcttcctgctatctcgcaaacc
ttgccaaaggaactctctaagttcttcgggcgtcttgacatgttcaagcc
tgctcctgaatggcgcaagcttacgtggggcattgaggccactctcatgg
gccccgagcttaaccgtgttccatcgtccacgttcgccaaggtagagaag
ggagcggcgggcaaatggttcgagttcttgccatacatgaccatcgctcc
aagtagcttggaaggcactc

Plasmid P2558 is able to transform Clitopilus passeckerianus to Hygromycin resistance but may not induce RNA interference.

Transformation of Clitopilus passeckerianus DSM1602

Clitopilus passeckerianus DSM1602 was obtained from German Collection of Microorganisms and Cell Cultures DSMZ, 38124 Braunschweig. This strain can easily be transformed to Hygromycin resistance with plasmid pPHT1 (Kilaru et al. 2009). Plasmids P2558 and P2543 Hairpin, derived from plasmid pPHT1, have been transformed into protoplasts as follows.

For the preparation of an inoculum, 1 well grown colony of DSM1602 is mechanically shared in H₂O to hyphal fragments of 5-10 cells in average and used for inoculation of 100 ml moist broken maize corns in a glas flask closed with a cotton plug. After incubation at 25° C. for 14 days the hyphal mesh is harvested by adding 50 ml of H₂O and vigorous shaking at 230 rpm for 2 hrs. The suspension containing broken hyphal fragments is filtered to separate from maize corns and used as inoculum for preparing liquid cultures. Subsequently, mycelium for protoplasting was grown for 3 days at 27° C. by inoculating 100 ml of medium containing corn steep liquor and glucose as carbon and nitrogen sources with 10 ml of DSM1602 inoculum. After cultivation for 3 days at 27° C. and 230 rpm the mycelium was harvested by zentrifugation. Protoplasting was performed in protoplasting solution (MgSO₄/PO₄ buffer pH 6.8 containing glucanex) at 27° C. and 230 rpm for 2 hrs. Sucrose/CaCl₂/Tris buffer pH 7.5 was used to wash protoplasts twice by centrifugation and to concentrate protoplasts to a density of 10⁸/ml.

For the transformation procedure, 50 μl of protoplast suspension was mixed with 5-10 μg of plasmid DNA and 25 μl of 36% PEG mix (polyethyleneclycol 4′000 in CaCl₂/Tris buffer pH 7.5). After incubating for 20 min. on crushed ice 200 μl of PEG mix was added and the suspension incubated for 5 min. at room temperature. In addition to transformation samples containing either plasmid P2543_Hairpin or the control P2558 also samples without DNA have been prepared to control the selection process. Selection of transformants was performed on potato dextrose agar containing 100 μg/ml Hygromycin. Selected transformants showing normal phenotypes with regard to growth speed and morphology have been used for analysis of pleuromutilin productivity in shake flask fermentations and transcription of the diterpen synthase gene to test for a correlation between Pleuromutilin productivity and expression of the diterpene synthase gene.

Quantitative PCR Expression Analysis of the Diterpene Synthase of Clitopilus passeckerianus

In order to quantify the expression pattern of diterpene synthase gene in transformants carrying the RNAi construct as well as control strains without this construct quantitative PCR analysis was performed (ABI7900 HT, Applied Biosystems) as described in Example 3.

RNA from mycelia of C. passeckarianus was collected by filtration on sterile cloth. The wet mycelium was flash-frozen in liquid nitrogen and ground to a powder using a ball mill. Total RNA was extracted using the TRIZOL® Reagent (Invitrogen) extraction protocol. All procedures were performed according to the manufacturers protocols. After extraction the RNA samples were purified with RNeasy® columns (Qiagen) and an on-column DNase digest was performed using the RNase-Free DNase Set Kit (Qiagen). RNA was re-suspended in DEPC-treated, sterile, destilled water and its concentration was measured by spectrophotometry (Nanodrop 1000, Peqlab). The quality of the RNA was checked by Bioanalyzer-measurements (Agilent) using the RNA 6000 Nano Assay (Agilent). Subsequent reverse transcription was performed using the High Capacity cDNA Archive Kit (Applied Biosytems).

Two independent primer sets for the diterpene synthase gene were designed using the Primer Express software (Applied Biosystems). The forward primer Cp_dts_U1 (5′-GTTACAGAGTTTGAGGCACCTACCT-3′) (SEQ ID NO: 56) and the reverse primer Cp_dts_L1 (5′-CGTGGAGGAGCGACATAAGG-3′) (SEQ ID NO: 57) which cover positions 997-1021 and 1096-1077, respectively, as well as the forward primer Cp_DTS_U2 (5′-AATCGTCAAGATCGCCACTTATG-3′) (SEQ ID NO: 70) and the reverse primer Cp_DTS_L2 (5′-GAGTACCATTCTGATACATTCCATTTG-3′) (SEQ ID NO: 71) which cover positions 1125-1147 and 1214-1188 of the spliced diterpene synthase messenger RNA sequence were designed. As a control, the act gene of C. passeckerianus was used for the design of the forward primer Cp_act_U1 (5′-TGATGGTCAAGTTATCACGATTGG-3′) (SEQ ID NO: 50) and the reverse primer Cp_act_L1 (5′-GAGTTGTAAGTGGTTTCGTGAATACC-3′) (SEQ ID NO: 51) which yield a 119-bp amplicon. For PCR reactions the SYBR Green Mastermix (Applied Biosystems) was used. The reaction was performed according to the manufacturers protocol using 12 μl reactions which each contained 12 ng of template cDNA. A no template control (NTC), with no added template RNA to control for any contaminants in reagents for each pair of primers was included. The good efficiency of all primer sets used was validated by standard curves.

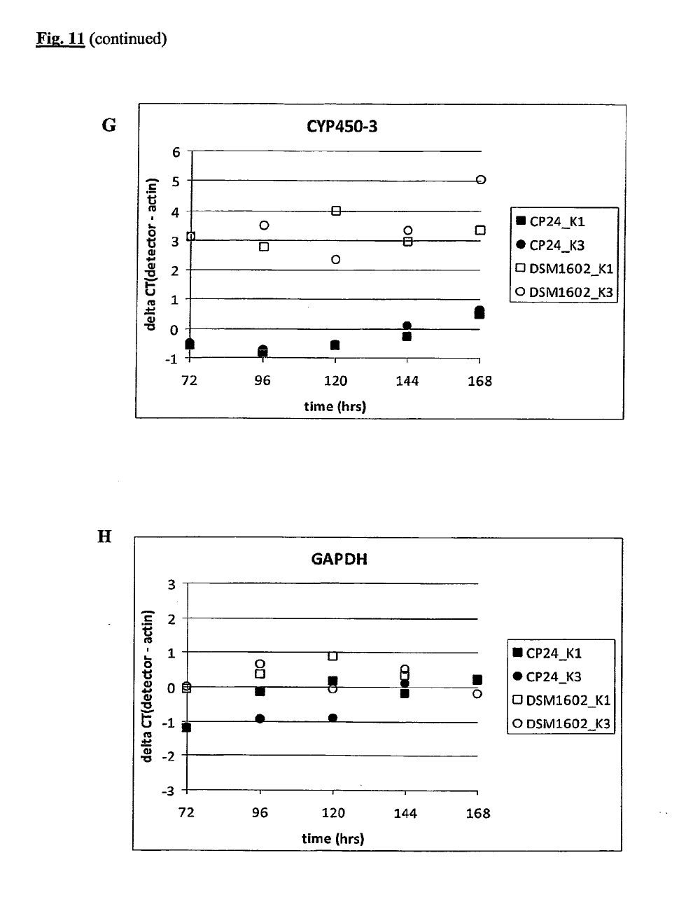

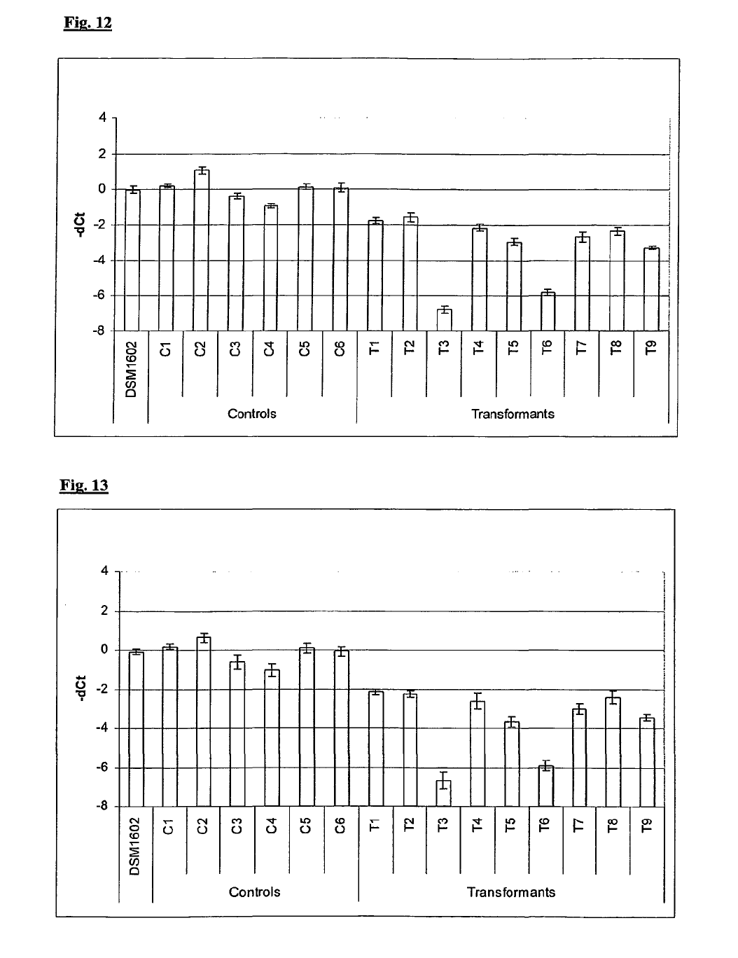

The results with both primer pairs used clearly confirm a lower level (about 4-fold) of diterpene synthase transcription in all strains transformed with the RNAi construct (c.f. FIG. 12; FIG. 13). Transformants (T1-T9) containing plasmid P2543_Hairpin obviously show a significantly reduced transcription of the diterpene synthase compared to transformants containing plasmid P2558 (C1-C6) and to parental strain DSM1602.

Productivity of Clitopilus passeckerianus DSM1602 Transformants

Pleuromutilin productivity of selected transformants has been analyzed in shake flask fermentations using Medium M2 containing corn steep liquor, glucose and butyloleat as carbon and nitrogen sources.

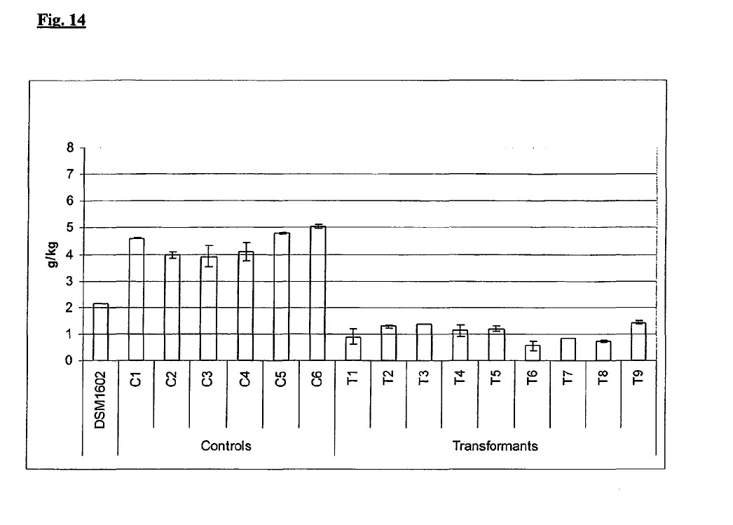

As consequence of RNA interference transformants (T1-T9) containing plasmid P2543_Hairpin obviously show a significantly reduced pleuromutilin productivity compared to transformants containing plasmid P2558 (C1-C6) and to parental strain DSM1602 (c.f. FIG. 14).

It can be concluded from this data that the diterpene synthase of Clitopilus passeckerianus has pleuromutilin synthase activity.

### Particular Embodiments

In the following section, the present invention is illustrated by several embodiments which are, however, not intended to limit the scope of the present invention.
1. An isolated polypeptide comprising an amino acid sequence,
   which amino acid sequence comprises one, particularly two, especially three sequences selected from the group consisting of
      a1) a sequence having at least 50% sequence identity to SEQ ID NO: 1;
      a2) a sequence having at least 40% sequence identity to SEQ ID NO: 2; and
      a3) a sequence having at least 50% sequence identity to SEQ ID NO: 3,
   and which amino acid sequence comprises at least one sequence selected from the group consisting of
      b1) a sequence having at least 25% sequence identity to SEQ ID NO: 4;
      b2) a sequence having at least 15% sequence identity to SEQ ID NO: 7;
      b3) a sequence having at least 45% sequence identity to SEQ ID NO: 5; and
      b4) a sequence having at least 45% sequence identity to SEQ ID NO: 6.
2. The isolated polypeptide of embodiment 1, wherein said amino acid sequence comprises any group of sequences selected from the following groups of sequences as defined in embodiment 1
   a1, b1; a1, b2; a1, b3; a1, b4; a1, b1, b2; a1, b1, b3; a1, b1, b4; a1, b2, b3; a1, b2, b4; a1, b3, b4; a1, b1, b2, b3; a1, b1, b2, b4; a1, b1, b3, b4; a1, b2, b3, b4; a1, b1, b2, b3, b4;
   a2, b1; a2, b2; a2, b3; a2, b4; a2, b1, b2; a2, b1, b3; a2, b1, b4; a2, b2, b3; a2, b2, b4; a2, b3, b4; a2, b1, b2, b3; a2, b1, b2, b4; a2, b1, b3, b4; a2, b2, b3, b4; a2, b1, b2, b3, b4;
   a3, b1; a3, b2; a3, b3; a3, b4; a3, b1, b2; a3, b1, b3; a3, b1, b4; a3, b2, b3; a3, b2, b4; a3, b3, b4; a3, b1, b2, b3; a3, b1, b2, b4; a3, b1, b3, b4; a3, b2, b3, b4; a3, b1, b2, b3, b4.
3. An isolated polypeptide comprising an amino acid sequence,
   which amino acid sequence comprises
      a1) a sequence having at least 50% sequence identity to SEQ ID NO: 1; and
      a2) a sequence having at least 40% sequence identity to SEQ ID NO: 2; and, optionally,
      a3) a sequence having at least 50% sequence identity to SEQ ID NO: 3,
   and which amino acid sequence comprises at least one sequence selected from the group consisting of
      b1) a sequence having at least 25% sequence identity to SEQ ID NO: 4;
      b2) a sequence having at least 15% sequence identity to SEQ ID NO: 7;
      b3) a sequence having at least 45% sequence identity to SEQ ID NO: 5; and
      b4) a sequence having at least 45% sequence identity to SEQ ID NO: 6.
4. The isolated polypeptide according to any one of embodiments 1 or 3, wherein said amino acid sequence comprises any group of sequences selected from the following groups of sequences as defined in embodiment 1
   a1, a2, b1; a1, a2, b2; a1, a2, b3; a1, a2, b4;
   a1, a2, b1, b2; a1, a2, b1, b3; a1, a2, b1, b4; a1, a2, b2, b3; a1, a2, b2, b4; a1, a2, b3, b4;
   a1, a2, b1, b2, b3; a1, a2, b1, b2, b4; a1, a2, b1, b3, b4; a1, a2, b2, b3, b4;
   a1, a2, b1, b2, b3, b4.
5. The isolated polypeptide according to any one of embodiments 1 or 3,
   wherein said amino acid sequence comprises any group of sequences selected from the following groups of sequences as defined in embodiment 1
      a1, a2, a3, b1; a1, a2, a3, b2; a1, a2, a3, b3; a1, a2, a3, b4;
      a1, a2, a3, b1, b2; a1, a2, a3, b1, b3; a1, a2, a3, b1, b4; a1, a2, a3, b2, b3; a1, a2, a3, b2, b4; a1, a2, a3, b3, b4;
      a1, a2, a3, b1, b2, b3; a1, a2, a3, b1, b2, b4; a1, a2, a3, b1, b3, b4; a1, a2, a3, b2, b3, b4;
      a1, a2, a3, b1, b2, b3, b4.
6. The polypeptide according to any one of embodiments 1-5, which comprises two sequences, particularly three sequences, especially four sequences of b1, b2, b3, and b4.
7. The polypeptide according to any one of embodiments 1-6, wherein the sequence defined in a1 has at least 55%, 60%, 65%, 70%, 75%, 80%, 85%, 90%, 92%, 95%, 97%, or 99%, particularly 100% sequence identity to SEQ ID NO: 1.
8. The polypeptide according to any one of embodiments 1-7, wherein the sequence defined in a2 has at least 45%, 50%, 55%, 60%, 65%, 70%, 75%, 80%, 85%, 90%, 92%, 95%, 97%, or 99%, particularly 100% sequence identity to SEQ ID NO: 2.
9. The polypeptide according to any one of embodiments 1-8, wherein the sequence defined in a3 has at least 55%, 60%, 65%, 70%, 75%, 80%, 85%, 90%, 92%, 95%, 97%, or 99%, particularly 100% sequence identity to SEQ ID NO: 3.
10. The polypeptide according to any one of embodiments 1-9, wherein the sequence defined in b1 has at least 30%, 35%, 40%, 45%, 50%, 55%, 60%, 65%, 70%, 75%, 80%, 85%, 90%, 92%, 95%, 97%, or 99%, particularly 100% sequence identity to SEQ ID NO: 4.
11. The polypeptide according to any one of embodiments 1-10, wherein the sequence defined in b2 has at least 20%, 25%, 30%, 35%, 40%, 45%, 50%, 55%, 60%, 65%, 70%, 75%, 80%, 85%, 90%, 92%, 95%, 97%, or 99%, particularly 100% sequence identity to SEQ ID NO: 7.
12. The polypeptide according to any one of embodiments 1-11, wherein the sequence defined in b3 has at least 50%, 55%, 60%, 65%, 70%, 75%, 80%, 85%, 90%, 92%, 95%, 97%, or 99%, particularly 100% sequence identity to SEQ ID NO: 5.
13. The polypeptide according to any one of embodiments 1-12, wherein the sequence defined in b4 has at least 50%, 55%, 60%, 65%, 70%, 75%, 80%, 85%, 90%, 92%, 95%, 97%, or 99%, particularly 100% sequence identity to SEQ ID NO: 6.
14. The isolated polypeptide according to embodiment 5 comprising the sequences a1, a2, a3, b1, b2, b3 and b4, wherein the sequences a1, a2, a3, b1, b2, b3 and b4 have the following sequence identities:
   a1 has at least 60% sequence identity to SEQ ID NO: 1, a2 has at least 50% sequence identity to SEQ ID NO: 2, a3 has at least 60% sequence identity to SEQ ID NO: 3, b1 has at least 35% sequence identity to SEQ ID NO: 4, b2 has at least 25% sequence identity to SEQ ID NO: 7, b3 has at least 55% sequence identity to SEQ ID NO: 5, and b4 has at least 55% sequence identity to SEQ ID NO: 6, more preferably wherein
   a1 has at least 70% sequence identity to SEQ ID NO: 1, a2 has at least 60% sequence identity to SEQ ID NO: 2, a3 has at least 70% sequence identity to SEQ ID NO: 3, b1 has at least 45% sequence identity to SEQ ID NO: 4, b2 has at least 35% sequence identity to SEQ ID NO: 7, b3 has at least 65% sequence identity to SEQ ID NO: 5, and b4 has at least 65% sequence identity to SEQ ID NO:6, and even more preferably wherein
   a1 has at least 80% sequence identity to SEQ ID NO: 1, a2 has at least 70% sequence identity to SEQ ID NO: 2, a3 has at least 80% sequence identity to SEQ ID NO: 3, b1 has at least 55% sequence identity to SEQ ID NO: 4, b2 has at least 45% sequence identity to SEQ ID NO: 7, b3 has at least 75% sequence identity to SEQ ID NO: 5, and b4 has at least 75% sequence identity to SEQ ID NO: 6.
15. The isolated polypeptide according to any one of embodiments 1-14, wherein at least five, particularly at least six, especially all seven of SEQ ID NOs: 1-7 are of Clitopilus passeckerianus origin.
16. The isolated polypeptide according to any one of embodiments 1-15, wherein the molecular weight of the polypeptide is between 90 kDa and 140 kDa, particularly between 100 kDa and 130 kDa, especially between 105 kDa and 120 kDa, and/or
   wherein the polypeptide comprises an amino acid sequence which amino acid sequence comprises a sequence having at least 70% sequence identity to SEQ ID NO: 9, more preferably at least 80%, even more preferably at least 85%, or even at least 90%, such as even more preferably at least 95% sequence identity to SEQ ID NO: 9.
17. The isolated polypeptide according to any one of embodiments 1-16, wherein said polypeptide is a diterpene synthase, particularly a pleuromutilin synthase, and/or wherein said polypeptide has diterpene synthase activity, particularly pleuromutilin synthase activity.
18. The isolated polypeptide according to any one of embodiments 1-17, wherein the polypeptide is involved in the biosynthetic pathway for producing pleuromutilin.
19. The isolated polypeptide according to any one of embodiments 1-18, wherein the polypeptide is capable of catalyzing the conversion of geranylgeranyl pyrophosphate into a pleuromutilin precursor, particularly into a compound according to formula (I).
20. The isolated polypeptide according to any one of embodiments 1-19, wherein said polypeptide is derivable from a fungal host, particularly a fungus from the division basidomycota, more particularly from the order agaricales, even more particularly from the family entolomataceae;
   in particular wherein said polypeptide is derivable from the genus Clitopilus or from the genus Pleurotus;
   especially wherein said polypeptide is derivable from any one of Clitopilus scyphoides, Clitopilus prunulus, Clitopilus hobsonii, Clitopilus pseudo-pinsitus, Clitopilus pinsitus and Clitopilus passeckerianus, in particular from Clitopilus pinsitus or Clitopilus passeckerianus.
21. An isolated nucleic acid molecule comprising
   A) a nucleotide sequence encoding a polypeptide according to any one of embodiments 1 to 20 or a polypeptide of SEQ ID NO: 9,
   B) a nucleotide sequence which is
      a) the sequence of SEQ ID NO: 8; or
      a′) the sequence of SEQ ID NO: 15 or the sequence complementary thereto; or
      b) a partial sequence of a sequence defined in a′), which partial sequence encodes a diterpene synthase; or
      c) a sequence which encodes a diterpene synthase and has at least 40% sequence identity to a sequence defined in a′) or has at least 60% sequence identity to the sequence defined in a) or the partial sequence defined in b); or
      d) a sequence which encodes a diterpene synthase and which is degenerate as a result of the genetic code to a sequence defined in any one of a), a′), b) and c); or
      e) a sequence which encodes a diterpene synthase and which is capable of hybridizing to SEQ ID NO: 8 and/or SEQ ID NO: 13 under stringent conditions,
   C) at least 18 consecutive nucleotides of a nucleotide sequence as defined in item B, and/or
   D) at least 18 consecutive nucleotides and capable of hybridizing to a nucleic acid molecule having a nucleotide sequence as defined in item A or item B under stringent conditions.
22. An isolated nucleic acid molecule comprising a nucleotide sequence encoding a polypeptide according to any one of embodiments 1 to 20 or a polypeptide of SEQ ID NO: 9.
23. An isolated nucleic acid molecule comprising a nucleotide sequence which is
   a) the sequence of SEQ ID NO: 8 or the sequence complementary thereto; or
   a′) the sequence of SEQ ID NO: 15 or the sequence complementary thereto; or
   b) a partial sequence of the sequence defined in a) or a′), which partial sequence encodes a polypeptide having diterpene synthase activity; or
   c) a sequence which encodes a polypeptide having diterpene synthase activity and has at least 40% sequence identity to a sequence defined in a) or a′) or has at least 60% sequence identity to the partial sequence defined in b); or
   d) a sequence which encodes a polypeptide having diterpene synthase activity and which is degenerate as a result of the genetic code to a sequence defined in any one of a), a′), b) and c); or
   e) a sequence which encodes a polypeptide having diterpene synthase activity and which is capable of hybridizing to a sequence defined in any one of a), a′), b) and c), particularly to SEQ ID NO: 8 and/or SEQ ID NO: 13, under stringent conditions,
24. An isolated nucleic acid molecule comprising a nucleotide sequence which is
   a) the sequence of SEQ ID NO: 15; or
   b) the sequence complementary thereto; or
   c) a sequence which encodes a polypeptide having diterpene synthase activity and has at least 40% sequence identity to the sequence defined in a) or b); or
   d) a sequence which encodes a polypeptide having diterpene synthase activity and which is degenerate as a result of the genetic code to a sequence defined in any one of a), b) and c); or
   e) a sequence which encodes a polypeptide having diterpene synthase activity and which is capable of hybridizing to a sequence defined in any one of a), b), c) and d), particularly to SEQ ID NO: 15, under stringent conditions.
25. An isolated nucleic acid molecule comprising a nucleotide sequence which is
   a) the sequence of SEQ ID NO: 15, which sequence encodes a gene cluster involved in a biosynthetic pathway for producing a diterpene; or
   b) the sequence of SEQ ID NO: 15, which sequence encodes a gene cluster involved in a biosynthetic pathway for producing pleuromutilin; or
   c) a sequence which encodes a polypeptide having diterpene synthase activity and has at least 40% sequence identity to the sequence defined in a) or b); or
   d) a sequence which encodes a polypeptide having diterpene synthase activity and which is degenerate as a result of the genetic code to a sequence defined in any one of a), b) and c); or
   e) a sequence which encodes a polypeptide having diterpene synthase activity and which is capable of hybridizing to SEQ ID NO: 15.
26. The isolated nucleic acid molecule according any one of embodiments 24 and 25, wherein said nucleic acid molecule comprises a gene cluster comprising nucleic acid sequences that encode for polypeptides which are capable of catalyzing the conversion of geranylgeranyl pyrophosphate into pleuromutilin, particularly that are capable of catalyzing the conversion of farnesyl diphosphate into pleuromutilin.
27. An isolated nucleic acid molecule comprising a nucleotide sequence which is
   a) the sequence of SEQ ID NO: 8; or
   b) the sequence of SEQ ID NO: 13; or
   c) a sequence which encodes a polypeptide having diterpene synthase activity and has at least 60% sequence identity to the sequence defined in a) or b); or
   d) a sequence which encodes a polypeptide having diterpene synthase activity and which is degenerate as a result of the genetic code to a sequence defined in any one of a), b) and c); or
   e) a sequence which encodes a polypeptide having diterpene synthase activity and which is capable of hybridizing to a sequence defined in any one of a), b), c) and d) under stringent conditions.
28. An isolated nucleic acid molecule comprising a nucleotide sequence which is
   a) a partial sequence of SEQ ID NO: 15, which sequence encodes a polypeptide having diterpene synthase activity; or
   b) a partial sequence of SEQ ID NO: 15, which sequence encodes a polypeptide having pleuromutilin synthase activity; or
   c) a sequence which encodes a polypeptide having diterpene synthase activity and has at least 60% sequence identity to the partial sequence defined in a) or b); or
   d) a sequence which encodes a polypeptide having diterpene synthase activity and which is degenerate as a result of the genetic code to a partial sequence defined in any one of a), b) and c); or
   e) a sequence which encodes a polypeptide having diterpene synthase activity and which is capable of hybridizing to a sequence defined in any one of a), b), c) and d) under stringent conditions,
29. The isolated nucleic acid molecule according any one of embodiments 21, 23, 24, 25 27 and 28, wherein said at least 40% sequence identity in item c) is at least 45%, 50%, 55%, particularly at least 60%, 65%, 70%, especially at least 75%, 80%, 85%, more particularly at least 90%, 92%, 95%, 97%, 99%, particularly 100% sequence identity; and/or
   wherein said at least 60% sequence identity in item c) is at least 65%, 70%, 75%, particularly at least 80%, 85%, especially at least 90%, 92%, more particularly at least 95%, 97%, 99%, particularly 100% sequence identity.
30. The nucleic acid molecule according to any one of embodiments 21-29, wherein said nucleic acid molecule is derivable from a fungal host, particularly a fungus from the division basidomycota, more particularly from the order agaricales, even more particularly from the family entolomataceae;
   in particular wherein said nucleic acid molecule is derivable from Clitopilus or from Pleurotus;
   especially wherein said nucleic acid molecule is derivable from any one of Clitopilus scyphoides, Clitopilus prunulus, Clitopilus hobsonii, Clitopilus pseudo-pinsitus, Clitopilus pinsitus and Clitopilus passeckerianus, in particular from Clitopilus pinsitus or Clitopilus passeckerianus.
31. The nucleic acid molecule according to any one of embodiments 21 to 30, wherein the sequence which encodes a polypeptide having pleuromutilin synthase activity is
   i) a sequence which encodes a diterpene synthase; and/or
   ii) a sequence which encodes a polypeptide having pleuromutilin synthase activity; and/or
   ii) a sequence which encodes a pleuromutilin synthase,
   particularly wherein said polypeptide having diterpene synthase activity is capable of catalyzing the conversion of geranylgeranyl pyrophosphate into a pleuromutilin precursor, especially into a compound according to formula (I).
32. An isolated nucleic acid molecule comprising at least 18, 19, 20, 25, particularly at least 30, 35, 40, 45, particularly at least 50, 55, 60, 65, particularly at least 70, 75, 80, 85, 90, 95, particularly at least 100, 150, 200, 250, particularly at least 300, 350, 400, 450, or 500 consecutive nucleotides of a sequence as defined in any one of embodiments 22 to 31; especially wherein the nucleic acid molecule further comprises a detectable label.
33. An isolated nucleic acid molecule comprising at least 18, 19, 20, 25, particularly at least 30, 35, 40, 45, particularly at least 50, 55, 60, 65, particularly at least 70, 75, 80, 85, 90, 95, particularly at least 100, 150, 200, 250, particularly at least 300, 350, 400, 450, or 500 consecutive nucleotides, wherein said nucleic acid molecule is capable of hybridizing to a nucleic acid molecule as defined in any one of embodiments 22 to 31 under stringent conditions; especially wherein the nucleic acid molecule further comprises a detectable label.
34. A polypeptide encoded by a nucleic acid molecule according to any one of embodiments 21 and 23 to 31.
35. The polypeptide according to embodiment 34, further defined as in any one of embodiments 1 to 20.
36. A vector comprising a nucleic acid molecule as defined in any one of embodiments 21 to 31, or a vector comprising a nucleic acid sequence as defined in any one of embodiments 21 to 31.
37. A non-naturally-occurring host selected from a cell, tissue and non-human organism, said host comprising at least one nucleic acid molecule comprising a nucleotide sequence as defined in any one of embodiments 21 to 31, particularly wherein said nucleotide sequence encodes a polypeptide having diterpene synthase activity, particularly a diterpene synthase or pleuromutilin synthase, especially wherein said polypeptide having diterpene synthase activity is capable of catalyzing the conversion of geranylgeranyl pyrophosphate into a pleuromutilin precursor, in particular into a compound according to formula (I).
38. A non-naturally-occurring host selected from a cell, tissue and non-human organism, said host comprising at least one vector according to embodiment 36.
39. A non-naturally-occurring host selected from a cell, tissue and non-human organism, said host comprising at least one nucleic acid molecule comprising a nucleotide sequence as defined in any one of embodiments 21 to 31,
   particularly wherein said nucleotide sequence encodes a polypeptide having diterpene synthase activity, particularly a diterpene synthase or pleuromutilin synthase, especially wherein said polypeptide having diterpene synthase activity is capable of catalyzing the conversion of geranylgeranyl pyrophosphate into a pleuromutilin precursor, in particular into a compound according to formula (I), and said host comprising at least one vector according to embodiment 36.
40. The host according to any one of embodiments 37 to 39, wherein said host is capable of producing a pleuromutilin precursor, in particular a compound according to formula (I).
41. The host according to any one of embodiments 37 to 40, wherein said host is capable of producing a diterpene or diterpenoid, particularly pleuromutilin.
42. The host according to any one of embodiments 37 to 41, wherein a corresponding naturally-occurring host selected from a cell, tissue and non-human organism not comprising said at least one said nucleic acid molecule and/or vector is capable of
   (i) producing a compound according to formula (I), and/or
   (ii) producing pleuromutilin.
43. The host according to any one of embodiments 37 to 41, wherein a corresponding naturally-occurring host selected from a cell, tissue and non-human organism not comprising said at least one said nucleic acid molecule and/or vector is incapable of
   (i) producing a compound according to formula (I), and/or
   (ii) producing pleuromutilin.
44. The host of according to any one of embodiments wherein said host is a fungal host, more particularly a fungus from the division basidomycota, even more particularly from the order agaricales, even more particularly from the family entolomataceae, in particular wherein said host is from the genus Clitopilus or from the genus Pleurotus;
   especially wherein said host is selected from the group consisting of Clitopilus scyphoides, Clitopilus prunulus, Clitopilus hobsonii, Clitopilus pseudo-pinsitus, Clitopilus pinsitus and Clitopilus passeckerianus, in particular from Clitopilus pinsitus or Clitopilus passeckerianus.
45. A method of producing a polypeptide according to any one of embodiments 1 to 20, 34 and 35, the method comprising
   (i) introducing into a host selected from a cell, tissue and non-human organism at least one nucleic acid molecule according to any one of embodiments 21 to 31 and/or at least one vector according to embodiment 36, and
   (ii) cultivating the host under conditions suitable for the production of the polypeptide,
   particularly wherein the method comprises a further step of (iii) recovering the polypeptide from the host.
46. A method of producing a polypeptide according to any one of embodiments 1 to 20, 34 and 35, the method comprising
   (i) introducing into a host selected from a cell, tissue and non-human organism at least one nucleic acid molecule according to any one of embodiments 21 to 31, and
   (ii) cultivating the host under conditions suitable for the production of the polypeptide,
   particularly wherein the method comprises a further step of (iii) recovering the polypeptide from the host.
47. A method of producing a polypeptide according to any one of embodiments 1 to 20, 34 and 35, the method comprising
   (i) introducing into a host selected from a cell, tissue and non-human organism at least one vector according to embodiment 36, and
   (ii) cultivating the host under conditions suitable for the production of the polypeptide,
   particularly wherein the method comprises a further step of (iii) recovering the polypeptide from the host.
48. A method of producing a polypeptide according to any one of embodiments 1 to 20, 34 and 35, the method comprising
   (i) introducing into a host selected from a cell, tissue and non-human organism at least one nucleic acid molecule according to any one of embodiments 21 to 31 and at least one vector according to embodiment 36, and
   (ii) cultivating the host under conditions suitable for the production of the polypeptide,
   particularly wherein the method comprises a further step of (iii) recovering the polypeptide from the host.
49. A method of producing pleuromutilin, the method comprising
   (i) introducing into a host selected from a cell, tissue and non-human organism a nucleic acid molecule according to any one of embodiments 21Ba′, 23, 24, and 25 and/or a vector comprising a nucleic acid molecule according to any one of embodiments 21Ba′, 23, 24, 25 and 26, and
   (ii) cultivating the host under conditions suitable for the production of pleuromutilin.
50. A method of producing pleuromutilin, the method comprising
   (i) introducing into a host selected from a cell, tissue and non-human organism a nucleic acid molecule according to any one of embodiments 21Ba′, 23, 24, 25 and 26, and
   (ii) cultivating the host under conditions suitable for the production of pleuromutilin.
51. A method of producing pleuromutilin, the method comprising
   (i) introducing into a host selected from a cell, tissue and non-human organism a vector comprising a nucleic acid molecule according to any one of embodiments 21Ba′, 23, 24, 25 and 26, and
   (ii) cultivating the host under conditions suitable for the production of pleuromutilin.
52. A method of producing pleuromutilin, the method comprising
   (i) introducing into a host selected from a cell, tissue and non-human organism a nucleic acid molecule according to any one of embodiments 21Ba′, 23, 24, and 25 and a vector comprising a nucleic acid molecule according to any one of embodiments 21Ba′, 23, 24, 25 and 26, and
   (ii) cultivating the host under conditions suitable for the production of pleuromutilin.
53. A method of producing a pleuromutilin precursor, in particular a compound according to formula (I), the method comprising
   (i) introducing into a host selected from a cell, tissue and non-human organism a nucleic acid molecule according to any one of embodiments 21 to 31 and/or a vector according to embodiment 36, and
   (ii) cultivating the host under conditions suitable for the production of said pleuromutilin precursor.
54. A method of producing a pleuromutilin precursor, in particular a compound according to formula (I), the method comprising
   (i) introducing into a host selected from a cell, tissue and non-human organism a nucleic acid molecule according to any one of embodiments 21 to 31, and
   (ii) cultivating the host under conditions suitable for the production of said pleuromutilin precursor.
55. A method of producing a pleuromutilin precursor, in particular a compound according to formula (I), the method comprising
   (i) introducing into a host selected from a cell, tissue and non-human organism a vector according to embodiment 36, and
   (ii) cultivating the host under conditions suitable for the production of said pleuromutilin precursor.
56. A method of producing a pleuromutilin precursor, in particular a compound according to formula (I), the method comprising
   (i) introducing into a host selected from a cell, tissue and non-human organism a nucleic acid molecule according to any one of embodiments 21 to 31 and a vector according to embodiment 36, and
   (ii) cultivating the host under conditions suitable for the production of said pleuromutilin precursor.
57. A method of altering the production of pleuromutilin in a host selected from a cell, tissue and non-human organism, wherein said host is capable of producing pleuromutilin and comprises at least one nucleic acid molecule comprising a nucleotide sequence as defined in any one of embodiments 21 to 31, the method comprising manipulating i) the expression, ii) the identity, or iii) both the expression and the identity of said at least one nucleic acid molecule;
   particularly wherein said method is
      a) a method of increasing the production of pleuromutilin, or
      b) a method of decreasing the production of pleuromutilin, in particular comprising disrupting or down-regulating said at least one nucleic acid molecule;
   especially wherein said host is a fungal host, more particularly a fungus from the division basidomycota, even more particularly from the order agaricales, even more particularly from the family entolomataceae.
58. A method of altering the production of pleuromutilin in a host selected from a cell, tissue and non-human organism, wherein said host is capable of producing pleuromutilin and comprises at least one nucleic acid molecule comprising a nucleotide sequence as defined in any one of embodiments 21 to 31, the method comprising manipulating the identity of said at least one nucleic acid molecule.
59. A method of altering the production of pleuromutilin in a host selected from a cell, tissue and non-human organism, wherein said host is capable of producing pleuromutilin and comprises at least one nucleic acid molecule comprising a nucleotide sequence as defined in any one of embodiments 21 to 31, the method comprising manipulating the expression of said at least one nucleic acid molecule.
60. The method according to any one of embodiments 57 to 59, wherein said method is a method of increasing the production of pleuromutilin.
61. The method according to any one of embodiments 57 to 59, wherein said method is a method of decreasing the production of pleuromutilin, in particular comprising disrupting or down-regulating said at least one nucleic acid molecule.
62. The method according to any one of embodiments 57 to 61, wherein said host is a fungal host, more particularly a fungus from the division basidomycota, even more particularly from the order agaricales, even more particularly from the family entolomataceae,
   in particular wherein said host is from the genus Clitopilus or from the genus Pleurotus;
   especially wherein said host is selected from group consisting of Clitopilus scyphoides, Clitopilus prunulus, Clitopilus hobsonii, Clitopilus pseudo-pinsitus, Clitopilus pinsitus and Clitopilus passeckerianus, in particular from Clitopilus pinsitus or Clitopilus passeckerianus.
63. Use of an isolated nucleic acid molecule according to any one of embodiments 21 to 31 in the production of pleuromutilin, wherein 2 to 50 nucleotides of the sequence of said nucleic acid molecule are divergent from a sequence of a gene cluster involved in the biosynthetic pathway for producing pleuromutilin comprised by a wild type organism capable of producing pleuromutilin; or wherein said nucleic acid molecule is a non-natural nucleic acid molecule.
64. Use of an isolated nucleic acid molecule according to any one of embodiments 21 to 31 in the production of a pleuromutilin precursor, wherein 2 to 50 nucleotides of the sequence of said nucleic acid molecule are divergent from a sequence encoding a diterpene synthase comprised by a wild type organism capable of producing pleuromutilin; or wherein said nucleic acid molecule is a non-natural nucleic acid molecule;
   particularly wherein said pleuromutilin precursor is a compound according to formula (I).
65. The use according to embodiment 63 or 64, wherein from 2 to 50 nucleotides are at least 3, 4, 5, 6, 7, 8, 9, 10, 11, 12, 13, 14, 15, 16, 17, 18, 19, 20, 25, 30, 35, 40, or 45 nucleotides,
   particularly from 3 to 100, more particularly from 5 to 80 nucleotides, even more particularly from 8 to 60 nucleotides.
66. Use of a host according to any one of embodiments 37 to 44, in the production of pleuromutilin or of a pleuromutilin precursor, particularly wherein said pleuromutilin precursor is a compound according to formula (I).
67. Use of an isolated nucleic acid molecule according to any one of embodiments 21 to 33, particularly of embodiments 32 or 33, for identifying one or more nucleic acids encoding a polypeptide having diterpene synthase activity, particularly pleuromutilin synthase activity and/or encoding a diterpene synthase, particularly a pleuromutilin synthase.
68. Use of an isolated nucleic acid molecule according to any one of embodiments 21 to 33, particularly of embodiments 32 or 33, in a method of isolating one or more nucleic acids encoding a polypeptide having diterpene synthase activity particularly pleuromutilin synthase activity and/or encoding a diterpene synthase, particularly a pleuromutilin synthase.
69. Use of an isolated nucleic acid molecule according to any one of embodiments 21 to 33, particularly of embodiments 32 or 33, in a method of isolating a polypeptide having diterpene synthase activity, particularly pleuromutilin synthase activity, and/or of isolating a diterpene synthase, particularly a pleuromutilin synthase.
70. The use of any one of embodiments 67 to 69, wherein said polypeptide having diterpene synthase activity or diterpene synthase is further defined as is the polypeptide of any one of embodiments 1 to 20.
71. The use of any one of embodiments 67 to 68, wherein said one or more nucleic acids are further defined as is the nucleic acid of any one of embodiments 21 to 33.
72. A method of the production of a pleuromutilin precursor, particularly of a compound according to formula (I), wherein the method
   A) is a method for the fermentative production of said precursor and particularly comprises the steps of
      (i) introducing into a host selected from a cell, tissue and non-human organism at least one nucleic acid molecule according to any one of embodiments 21 to 31 and/or at least one vector according to embodiment 36, and
      (ii) cultivating the host under conditions suitable for the fermentative production of said precursor;
   or
   B) is a method for the synthetic production of said precursor and comprises reacting geranylgeranylpyrophosphate with a polypeptide according to any one of claims 1 to 20, 34, and 35, or a polypeptide obtainable by a method of any one of embodiments 45 to 48.
73. A method of the production of a pleuromutilin precursor, particularly of a compound according to formula (I), wherein the method is a method for the fermentative production of said precursor and particularly comprises the steps of
   (i) introducing into a host selected from a cell, tissue and non-human organism at least one nucleic acid molecule according to any one of embodiments 21 to 31 and/or at least one vector according to embodiment 36, and
   (ii) cultivating the host under conditions suitable for the fermentative production of said precursor;
   particularly wherein the method comprises a further step of (iii) recovering said precursor from the host.
74. The method according to embodiment 72 or 73, wherein said host is a fungal host, more particularly a fungus from the division basidomycota, even more particularly from the order agaricales, even more particularly from the family entolomataceae,
   in particular wherein said host is from the genus Clitopilus or from the genus Pleurotus;
   especially wherein said host is selected from group consisting of Clitopilus scyphoides, Clitopilus prunulus, Clitopilus hobsonii, Clitopilus pseudo-pinsitus, Clitopilus pinsitus and Clitopilus passeckerianus, in particular from Clitopilus pinsitus or Clitopilus passeckerianus.
75. A method of the production of a pleuromutilin precursor, particularly of a compound according to formula (I), wherein the method is a method for the synthetic production of said precursor and comprises reacting geranylgeranylpyrophosphate with a polypeptide according to any one of claims 1 to 20, 34, and 35, or with a polypeptide obtainable by a method of any one of embodiments 45 to 48.
76. An isolated compound according to formula (I).
77. An isolated compound according to formula (I), wherein said compound is obtainable by a method of any one of embodiments 53 to 56 or 72 to 75.
78. A method for the production of a pleuromutilin antibiotic, wherein the method comprises a step of reacting
   i) a pleuromutilin precursor obtained by a method according to any one of embodiments 53 to 56 and 72 to 75;
   ii) a pleuromutilin precursor obtained by a use of embodiment 64; or
   iii) an isolated compound according to embodiment 76 or 77,
   the method optionally comprising further reaction steps to produce said pleuromutilin antibiotic.
79. Use of
   i) a pleuromutilin precursor obtained by a method according to any one of embodiments 53 to 56 and 72 to 75;
   ii) a pleuromutilin precursor obtained by a use of embodiment 64; or
   iii) an isolated compound according to embodiment 76 or 77;
   in the production of a pleuromutilin antibiotic, particularly a pleuromutilin derivative.
80. A pleuromutilin obtained according to any one of embodiments 49 to 52, 63 and 66
   i) for use as a medicament; or
   ii) for use in a method of treating a bacterial infection, particularly an infection caused by a bacterium selected from the group consisting of Gram-positive bacteria particularly selected from staphylococci, streptococci, pneumococci and enterococci; Gram-negative bacteria particularly selected from the genera Neisseria, Haemophilus, Moraxella, Bordetella, Legionella, Leptospira; mycoplasmas; chlamydia; Gram-positive anaerobes and Gram-negative anaerobes; or
   iii) for use in treating a disorder or disease involving a bacterium selected from the group consisting of Gram-positive bacteria particularly selected from staphylococci, streptococci, pneumococci and enterococci; Gram-negative bacteria particularly selected from the genera Neisseria, Haemophilus, Moraxella, Bordetella, Legionella, Leptospira; mycoplasmas; chlamydia; Gram-positive anaerobes and Gram-negative anaerobes.
81. A pleuromutilin antibiotic obtained by a method of embodiment 78
   i) for use as a medicament; or
   ii) for use in a method of treating a bacterial infection, particularly an infection caused by a bacterium selected from the group consisting of Gram-positive bacteria particularly selected from staphylococci, streptococci, pneumococci and enterococci; Gram-negative bacteria particularly selected from the genera Neisseria, Haemophilus, Moraxella, Bordetella, Legionella, Leptospira; mycoplasmas; chlamydia; Gram-positive anaerobes and Gram-negative anaerobes; or
   iii) for use in treating a disorder or disease involving a bacterium selected from the group consisting of Gram-positive bacteria particularly selected from staphylococci, streptococci, pneumococci and enterococci; Gram-negative bacteria particularly selected from the genera Neisseria, Haemophilus, Moraxella, Bordetella, Legionella, Leptospira; mycoplasmas; chlamydia; Gram-positive anaerobes and Gram-negative anaerobes.

### REFERENCES

W. J. Cummings, M. Celerin, J. Crodian, L. K. Brunick, and M. E. Zolan (1999) Insertional mutagenesis in Coprinus cinereus: use of a dominant selectable marker to generate tagged, sporulation-defective mutants, Curr Genet 36: 371-382
D. M. Binninger, C. Skrzynia, P. J. Pukkila and L. A. Casselton (1987) DNA-mediated transformation of the Basidiomycete Coprinus cinereus, The EMBO Journal, p. 835-840, Vol 6, No. 4
Dewick P M. (2002) The biosynthesis of C-5-C-25 terpenoid compounds. Nat Prod Rep; 19(2):181-222
Kawaide H, Imai R, Sassa T, Kamiya Y. (1997) Ent-kaurene synthase from the fungus Phaeosphaeria sp. L487. cDNA isolation, characterization, and bacterial expression of a bifunctional diterpene cyclase in fungal gibberellin biosynthesis, J Biol Chem., 29; 272(35): 21706-12
Kilaru S., Catherine M. Collins, Amanda J. Hartley, Claire Burns, Gary D. Foster (2009a). Investigating dominant selection markers for Coprinopsis cinerea: a carboxin resistance system and re-evaluation of hygromycin and phleomycin resistance vectors, Current Genetics, p. 543-550, Vol. 55
Kilaru S, Collins C M, Hartley A J, Bailey A M, Foster G D. (2009b) Establishing molecular tools for genetic manipulation of the pleuromutilin-producing fungus Clitopilus passeckerianus. Appl Environ Microbiol, 75(22): 7196-204.
Hartley A J, de Mattos-Shipley K, Collins C M, Kilaru S, Foster G D, Bailey A M. (2009) Investigating pleuromutilin-producing Clitopilus species and related basidiomycetes. FEMS Microbiol Lett; 297(1):24-30.
Hu C, Zou Y. (2009) Mutilins Derivatives: From Veterinary to Human-used Antibiotics. Mini-Reviews in Medicinal Chemistry; 9(12):1397-1406
Hunt E. (2000) Pleuromutilin antibiotics. Drugs of the Future, 55(11): 1163-1168.
Ikeda H, Ishikawa J, Hanamoto A, Shinose M, Kikuchi H, Shiba T et al. (2003) Complete genome sequence and comparative analysis of the industrial microorganism Streptomyces avermitilis. Nature Biotechnology; 21(5):526-531
Keller N P, Turner G, Bennett J W. (2005) Fungal secondary metabolism—From biochemistry to genomics. Nature Reviews Microbiology; 3(12):937-947
Kuzuyama T. (2002) Mevalonate and nonmevalonate pathways for the biosynthesis of isoprene units. Bioscience Biotechnology and Biochemistry; 66(8):1619-1627
Lacalle R A, J A Tercero, and A Jiménez (1992), Cloning of the complete biosynthetic gene cluster for an aminonucleoside antibiotic, EMBO J.; 11(2): 785-792.
G H Jones and D A Hopwood (1984), Molecular cloning and expression of the phenoxazinone synthase gene from Streptomyces, J. Biol. Chem. 259: 14151-14157.
H Motamedi and C R Hutchinson (1987), Cloning and heterologous expression of a gene cluster for the biosynthesis of tetracenom, Proc Natl Acad Sci USA.; 84(13): 4445-4449.
Oliynyk M, Samborskyy M, Lester J B, Mironenko T, Scott N, Dickens S et al. (2007) Complete genome sequence of the erythromycin-producing bacterium Saccharopolyspora erythraea NRRL23338. Nature Biotechnology; 25(4):447-453
Thompson J. D., Gibson T. J., Plewniak F., Jeanmougin F. and Higgins D. G. (1997) The ClustalX windows interface: flexible strategies for multiple sequence alignment aided by quality analysis tools. Nucleic Acids Research 24:4876-4882
Toyomasu T, Kawaide H, Ishizaki A, Shinoda S, Otsuka M, Mitsuhashi W et al. (2000) Cloning of a full-length cDNA encoding ent-kaurene synthase from Gibberella fujikuroi: Functional analysis of a bifunctional diterpene cyclase. Bioscience Biotechnology and Biochemistry; 64(3):660-664.
Toyomasu T, Nakaminami K, Toshima H, Mie T, Watanabe K, Ito H, Matsui H, Mitsuhashi W, Sassa T, Oikawa H. (2004), Cloning of a gene cluster responsible for the biosynthesis of diterpene aphidicolin, a specific inhibitor of DNA polymerase alpha, Biosci Biotechnol Biochem., 68(1): 146-52.
Tsukagoshi T, Tokiwano T, Oikawa H. (2007) Studies on the later stage of the biosynthesis of pleuromutilin. Bioscience Biotechnology and Biochemistry; 71(12):3116-3121
Yao, Qingmei (2007) Biosynthetic Studies of Fungal Diterpene Antibiotics. Master Thesis, Oregon State University
CN 101318921
IN 2005CH00521
WO 2009075776

1.-15. (canceled)

16. An isolated polypeptide,
the polypeptide comprising an amino acid sequence which comprises
a sequence having at least 50% sequence identity to SEQ ID NO: 1,
a sequence having at least 40% sequence identity to SEQ ID NO: 2, and
at least one sequence selected from the group consisting of
i) a sequence having at least 15% sequence identity to SEQ ID NO: 7;
ii) a sequence having at least 25% sequence identity to SEQ ID NO: 4;
iii) a sequence having at least 45% sequence identity to SEQ ID NO: 5; and
iv) a sequence having at least 45% sequence identity to SEQ ID NO: 6,
wherein SEQ ID NOs: 1-2 and 4-7 are of Clitopilus passeckerianus origin and wherein said polypeptide is a diterpene synthase.

17. The isolated polypeptide according to claim 16, wherein said amino acid sequence further comprises a sequence having at least 50% sequence identity to SEQ ID NO: 3, wherein SEQ ID NO: 3 is of Clitopilus passeckerianus origin.

18. The isolated polypeptide according to claim 16, wherein the molecular weight of the polypeptide is between 90 kDa and 140 kDa.

19. The isolated polypeptide according to claim 16, wherein the polypeptide comprises an amino acid sequence which amino acid sequence comprises a sequence having at least 70% sequence identity to SEQ ID NO: 9.

20. The isolated polypeptide according to claim 16, wherein the polypeptide is capable of catalyzing the conversion of geranyl pyrophosphate into a pleuromutilin precursor.

21. The isolated polypeptide according to claim 20, wherein said pleuromutilin precursor is a compound according to formula (I).

22. The isolated polypeptide according to claim 16, wherein said polypeptide is derivable from a fungal host.

23. The isolated polypeptide according to claim 22, wherein said polypeptide is derivable from Clitopilus pinsitus or Clitopilus passeckerianus.

24. An isolated nucleic acid molecule comprising
a nucleotide sequence encoding a polypeptide according to claim 16 or a polypeptide of SEQ ID NO: 9.

25. An isolated nucleic acid molecule comprising a nucleotide sequence which
has at least 40% sequence identity to SEQ ID NO: 15 or the sequence complementary thereto; or
which has at least 60% sequence identity to SEQ ID NO: 8; or
which has at least 60% sequence identity to a partial sequence of SEQ ID NO: 15 or the sequence complementary thereto, which partial sequence encodes a diterpene synthase.

26. The isolated nucleic acid molecule of claim 25, wherein said nucleotide sequence is the sequence of SEQ ID NO: 8, or wherein said nucleotide sequence is the sequence of SEQ ID NO: 15 or the sequence complementary thereto.

27. The isolated nucleic acid molecule of claim 25, wherein said nucleotide sequence is degenerated from SEQ ID NO: 8, or SEQ ID NO: 15 or the sequence complementary thereto, or a partial sequence of SEQ ID NO: 15 or the sequence complementary thereto as a result of the genetic code.

28. An isolated nucleic acid molecule comprising a nucleotide sequence, which is capable of hybridizing to SEQ ID NO: 8, or SEQ ID NO: 13, or both under stringent conditions.

29. An isolated nucleic acid molecule comprising at least 18 consecutive nucleotides of a nucleotide sequence as defined in claim 25.

30. An isolated nucleic acid molecule comprising
(i) a nucleotide sequence encoding a polypeptide comprising an amino acid sequence which comprises:
a sequence having at least 50% sequence identity to SEQ ID NO: 1,
a sequence having at least 40% sequence identity to SEQ ID NO: 2, and
at least one sequence selected from the group consisting of
a) a sequence having at least 15% sequence identity to SEQ ID NO: 7;
b) a sequence having at least 25% sequence identity to SEQ ID NO: 4;
c) a sequence having at least 45% sequence identity to SEQ ID NO: 5; and
d) a sequence having at least 45% sequence identity to SEQ ID NO: 6, wherein SEQ ID NOs: 1-2 and 4-7 are of Clitopilus passeckerianus origin and wherein said polypeptide is a diterpene synthase
or a polypeptide of SEQ ID NO: 9; or
(ii) a nucleotide sequence which has at least 40% sequence identity to SEQ ID NO: 15 or the sequence complementary thereto; or
(iii) a nucleotide sequence which has at least 60% sequence identity to SEQ ID NO: 8; or
(iv) a nucleotide sequence which has at least 60% sequence identity to a partial sequence of SEQ ID NO: 15 or the sequence complementary thereto, which partial sequence encodes a diterpene synthase; or
(v) a nucleotide sequence, which is capable of hybridizing to SEQ ID NO: 8, or SEQ ID NO: 13, or both under stringent conditions.

31. The isolated nucleic acid molecule of claim 29, which is capable of hybridizing under stringent conditions to a nucleic acid molecule that comprises
(i) a nucleotide sequence encoding a polypeptide comprising an amino acid sequence which comprises:
a sequence having at least 50% sequence identity to SEQ ID NO: 1,
a sequence having at least 40% sequence identity to SEQ ID NO: 2, and
at least one sequence selected from the group consisting of
a) a sequence having at least 15% sequence identity to SEQ ID NO: 7;
b) a sequence having at least 25% sequence identity to SEQ ID NO: 4;
c) a sequence having at least 45% sequence identity to SEQ ID NO: 5; and
d) a sequence having at least 45% sequence identity to SEQ ID NO: 6, wherein SEQ ID NOs: 1-2 and 4-7 are of Clitopilus passeckerianus origin and wherein said polypeptide is a diterpene synthase
or a polypeptide of SEQ ID NO: 9; or
(ii) a nucleotide sequence which has at least 40% sequence identity to SEQ ID NO: 15 or the sequence complementary thereto; or
(iii) a nucleotide sequence which has at least 60% sequence identity to SEQ ID NO: 8; or
(iv) a nucleotide sequence which has at least 60% sequence identity to a partial sequence of SEQ ID NO: 15 or the sequence complementary thereto, which partial sequence encodes a diterpene synthase; or
(v) a nucleotide sequence, which is capable of hybridizing to SEQ ID NO: 8, or SEQ ID NO: 13, or both under stringent conditions.

32. The isolated nucleic acid molecule according to claim 30 or at least 18 consecutive nucleotides thereof, wherein said nucleic acid molecule is derivable from a fungal host.

33. The isolated nucleic acid molecule according to claim 30, wherein said nucleic acid molecule is derivable from Clitopilus pinsitus or Clitopilus passeckerianus.

34. A vector comprising a nucleic acid molecule of claim 30.

35. A non-naturally-occurring host selected from a cell, tissue and non-human organism, said host comprising at least one nucleic acid molecule of claim 30, or a vector comprising the nucleic acid.

36. The host according to claim 37, wherein said host is a fungal host.

37. A method of producing a polypeptide, the method comprising
(i) introducing into a host selected from a cell, tissue and non-human organism at least one nucleic acid molecule according to claim 30 or a vector comprising the at least one nucleic acid molecule;
(ii) cultivating the host under conditions suitable for the production of the polypeptide; and
(iii) recovering the polypeptide from the host.

38. A method of producing pleuromutilin, the method comprising
(i) introducing into a host selected from a cell, tissue and non-human organism a nucleic acid molecule having the sequence of SEQ ID NO: 15 or the sequence complementary thereto, or a vector comprising a nucleic acid molecule having the sequence of SEQ ID NO: 15 or the sequence complementary thereto, and
(ii) cultivating the host under conditions suitable for the production of pleuromutilin.

39. A method of altering the production of pleuromutilin in a host selected from a cell, tissue and non-human organism, wherein said host is capable of producing pleuromutilin and comprises at least one nucleic acid molecule of claim 30, the method comprising manipulating i) the expression, ii) the identity, or iii) both the expression and the identity of said at least one nucleic acid molecule.

40. The method of claim 39, wherein said method is
a) a method of increasing the production of pleuromutilin, or
b) a method of decreasing the production of pleuromutilin.

41. The method of claim 39, wherein said method of decreasing the production of pleuromutilin comprises disrupting or down-regulating said at least one nucleic acid molecule.

42. A method for the production of pleuromutilin or a pleuromutilin precursor, comprising using an isolated nucleic acid molecule of claim 30, wherein
a) in the production of pleuromutilin, 2 to 50 nucleotides of the sequence of said nucleic acid molecule are divergent from a sequence of a gene cluster involved in the biosynthetic pathway for producing pleuromutilin comprised by a wild type organism capable of producing pleuromutilin; or
b) in the production of a pleuromutilin precursor, 2 to 50 nucleotides of the sequence of said nucleic acid molecule are divergent from a sequence encoding a diterpene synthase comprised by a wild type organism capable of producing pleuromutilin.

43. The method of claim 41, wherein said pleuromutilin precursor is a compound according to formula (I).

44. A method for the production of pleuromutilin or of a pleuromutilin precursor, which method comprises using a host according to claim 34.

45. The method of claim 43, wherein said pleuromutilin precursor is a compound according to formula (I).

46. A method of identifying one or more nucleic acids encoding a polypeptide having diterpene synthase activity, which method comprises
using an isolated nucleic acid molecule according to claim 30 or at least 18 consecutive nucleotides thereof.

47. The method of claim 45, wherein said nucleic acid encoding a polypeptide having diterpene synthase activity encodes a diterpene synthase.

48. The method of claim 45, wherein said nucleic acid encoding a polypeptide having diterpene synthase activity encodes a pleuromutilin synthase.

49. A method of the production of a pleuromutilin precursor, wherein the method
is a method for the fermentative production of said precursor and comprises the steps of
(i) introducing into a host selected from a cell, tissue and non-human organism at least one nucleic acid molecule according to claim 30 or a vector comprising the at least one nucleic acid molecule or at least one vector comprising the nucleic acid molecule, and
(ii) cultivating the host under conditions suitable for the fermentative production of said precursor.

50. A method for synthetic production of a pleuromutilin precursor, wherein the method comprises reacting geranyl pyrophosphate with a polypeptide.

51. The method of claim 50, wherein the polypeptide reacted with geranyl pyrophosphate comprises:
a sequence having at least 50% sequence identity to SEQ ID NO: 1,
a sequence having at least 40% sequence identity to SEQ ID NO: 2, and
at least one sequence selected from the group consisting of
i) a sequence having at least 15% sequence identity to SEQ ID NO: 7;
ii) a sequence having at least 25% sequence identity to SEQ ID NO: 4;
iii) a sequence having at least 45% sequence identity to SEQ ID NO: 5; and
iv) a sequence having at least 45% sequence identity to SEQ ID NO: 6,
wherein SEQ ID NOs: 1-2 and 4-7 are of Clitopilus passeckerianus origin and wherein said polypeptide is a diterpene synthase.

52. The method of claim 50, wherein the polypeptide reacted with geranyl pyrophosphate is a polypeptide obtainable by a method comprising:
(a) introducing into a host selected from a cell, tissue and non-human organism at least one nucleic acid molecule or a vector comprising the at least one nucleic acid molecule, wherein the nucleic acid molecule comprises
(i) a nucleotide sequence encoding a polypeptide comprising an amino acid sequence which comprises:
a sequence having at least 50% sequence identity to SEQ ID NO: 1,
a sequence having at least 40% sequence identity to SEQ ID NO: 2, and
at least one sequence selected from the group consisting of
a) a sequence having at least 15% sequence identity to SEQ ID NO: 7;
b) a sequence having at least 25% sequence identity to SEQ ID NO: 4;
c) a sequence having at least 45% sequence identity to SEQ ID NO: 5; and
d) a sequence having at least 45% sequence identity to SEQ ID NO: 6, wherein SEQ ID NOs: 1-2 and 4-7 are of Clitopilus passeckerianus origin and wherein said polypeptide is a diterpene synthase or a polypeptide of SEQ ID NO: 9; or
(ii) a nucleotide sequence which has at least 40% sequence identity to SEQ ID NO: 15 or the sequence complementary thereto; or
(iii) a nucleotide sequence which has at least 60% sequence identity to SEQ ID NO: 8; or
(iv) a nucleotide sequence which has at least 60% sequence identity to a partial sequence of SEQ ID NO: 15 or the sequence complementary thereto, which partial sequence encodes a diterpene synthase; or
(v) a nucleotide sequence, which is capable of hybridizing to SEQ ID NO: 8, or SEQ ID NO: 13, or both under stringent conditions;
(b) cultivating the host under conditions suitable for the production of the polypeptide; and
(c) recovering the polypeptide from the host.

53. The method of claim 48, wherein the pleuromutilin precursor is a compound according to formula (I).
